(19) United States
(12) Patent Application Publication
(10) Pub. No.: US 2025/0114313 A1
(43) Pub. Date: Apr. 10, 2025

(54) OLIGO-BENZAMIDE ANALOGS AND THEIR USE IN CANCER TREATMENT

(71) Applicant: BOARD OF REGENTS, THE UNIVERSITY OF TEXAS SYSTEM, Austin, TX (US)

(72) Inventors: Ganesh RAJ, Dallas, TX (US); Ratna VADLAMUDI, San Antonio, TX (US); Jung-Mo AHN, Plano, TX (US); Xihui LIU, Dallas, TX (US)

(73) Assignee: BOARD OF REGENTS, THE UNIVERSITY OF TEXAS SYSTEM, Austin, TX (US)

(21) Appl. No.: 18/836,667

(22) PCT Filed: Feb. 10, 2023
(86) PCT No.: PCT/US2023/062375

§ 371 Date: Aug. 7, 2024

Related U.S. Application Data
(60) Provisional application No. 63/309,127, filed on Feb. 11, 2022.

(51) Int. Cl. A61K 31/167; A61K 31/47; A61P 35/00

(52) U.S. Cl. CPC A61K 31/167; A61K 31/47; A61P 35/00

(57) ABSTRACT

The present disclosure describes an oligobenzamide-based peptidomimetic agent with potent antiproliferative activity against triple negative breast cancer (TNBC), including TNBC cells representing all the known molecular subtypes. Lysosomal acid lipase A (LIPA) was identified as important for this agent's activity with the ER localization, not the lysosomal lipase function, of LIPA being critical. This agent can also block the growth in vitro and in vivo of multiple cancer types that have high basal level of ER stress and LIPA expression, including estrogen receptor-positive breast, pancreatic, glioblastoma and ovarian cancers. Thus, the inventors propose a new targeted strategy for TNBC and other rapidly proliferating cancers.

### PRIORITY CLAIM

This application claims benefit of priority to U.S. Provisional Application Ser. No. 63/309,127, filed Feb. 11, 2022, the entire contents of which are hereby incorporated by reference.

### FEDERAL FUNDING STATEMENT

This invention was made with government support under Grant No. R01-CA223828 awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Feb. 10, 2023, is named UTFFP0158WO.xml and is 56,033 bytes in size.

### FIELD OF THE INVENTION

The present disclosure relates in general to the field of peptidomimetics and specifically to compositions of matter and methods of their use in medical indications, such as cancer.

### BACKGROUND

Peptidomimetics (also known as peptide mimetics) are small organic molecules that do not possess a peptide backbone structure. However, they still retain a capability to interact with a target protein by arranging essential functional groups in a required three-dimensional pattern complimentary to a binding pocket in the target protein. Some peptidomimetics have been shown to bind hormone receptors in cancer cells and may be useful in treating these indications. However, there remains an unmet need to develop new peptidomimetics which are useful in the treatment of cancers through the modulation of alternative targets.

### SUMMARY

The present disclosure provides oligo-benzamide peptidomimetic compounds for use in the treatment and/or prevention of cancer, particularly breast cancer, including triple-negative breast cancer (TNBC). These small molecules include α-helix mimetics that represent helical segments in the target molecules. The oligo-benzamide peptidomimetic compounds modulate protein-protein, protein-peptide, or protein-drug interaction to exert a variety of physiological consequences. The oligo-benzamide peptidomimetic compounds also cause significant endoplasmic reticulum stress in cancer cells and may effectively shut down de novo protein synthesis, leading to cell death.

An aspect of the present disclosure is a method for treating a cancer characterized by having increased activity of Lysosomal lipase A (LIPA) comprising administering a therapeutically-effective amount of a composition comprising a compound of Formula I:

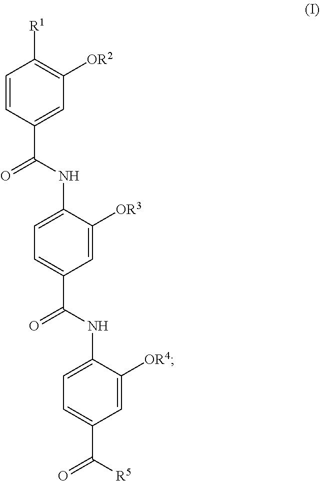

to a subject having a cancer with elevated LIPA expression.

Another aspect of the present disclosure is a method for inhibiting activity of Lysosomal lipase A (LIPA) in a subject having a cancer comprising administering a therapeutically-effective amount of a composition comprising a compound of Formula I:

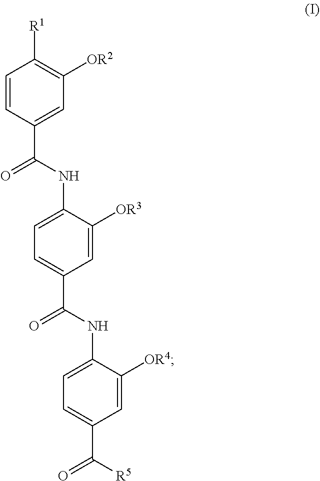

to the subject.

A further aspect of the present disclosure is a method for treating cancer in a subject in need thereof comprising testing a sample from the subject for the presence of elevated Lysosomal lipase A (LIPA) expression and administering a therapeutically-effective amount of a composition comprising a compound of Formula I:

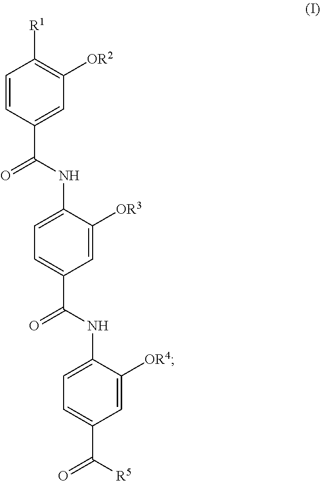

to a subject that provided a sample identified as having an elevated LIPA expression.

In embodiments, the compound of Formula I inhibits LIPA activity when contacted to the LXXLL motif on LIPA.

In several embodiments, the compound is a pharmaceutically acceptable salt of Formula I.

In various embodiments, R¹ is halogen, —NO₂, alkyl_{(c<12)}, substituted alkyl_{(C<12)}, amido_{(C<12)}, substituted amido_{(C<12)}, or —NHC(O)CH(R^{1a})NH₂, wherein: R^{1a} is aralkyl_{(C<18)}, substituted aralkyl_{(C<18)}, or the side chain of a canonical amino acid; R², R³, and R⁴ are each independently alkyl_{(C<12)}, substituted alkyl_{(C<12)}, aralkyl_{(C<18)}, or substituted aralkyl_{(C<18)}; and R⁵ is —OR^{5a} or —NHR^{5b}, wherein: R^{5a} is alkyl_{(C<12)} or substituted alkyl_{(C<12)}; R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or a group of the formula:

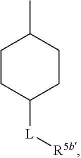

wherein; L is —CO₂— or —C(O)NR^{L}—, wherein: R^{L} hydrogen, alkyl_{(C<12)}, or substituted alkyl_{(C<12)}; R^{5b′} is aryl_{(C<12)}, aralkyl_{(C<18)}, heteroaryl_{(C<12)}, heteroaralkyl_{(C<18)}, or a substituted version of any of these groups.

In embodiments, the composition comprises the compound of Formula II:

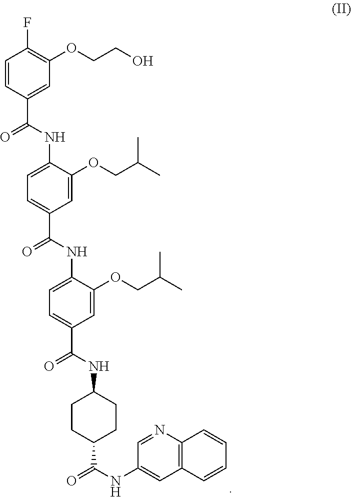 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

In several embodiments, the composition comprises the compound of Formula III:

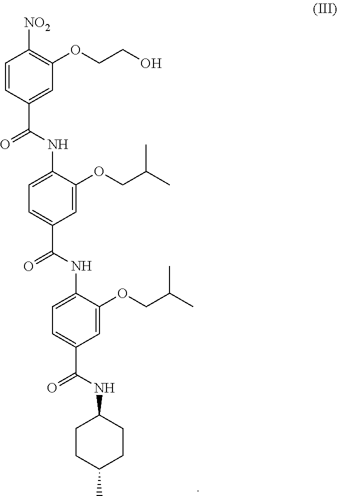 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

In several embodiments, the composition comprises the compound of Formula IV:

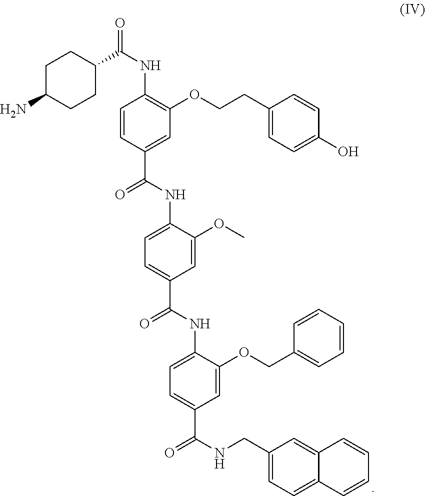 [SMILES: COc1cc(C(=O)Nc2ccc(C(=O)NCc3ccc4ccccc4c3)cc2OCc2ccccc2)ccc1NC(=O)c1ccc(NC(=O)[C@H]2CC[C@H](N)CC2)c(OCCc2ccc(O)cc2)c1]

In embodiments, the compound is a pharmaceutically acceptable salt of Formula II, Formula III, or Formula IV.

In various embodiments, the compound of Formula II, Formula III, or Formula IV inhibits LIPA activity when contacted to the LXXLL motif on LIPA.

In some embodiments, the subject is a mammal (e.g., a human). In several embodiments, the cancer is a therapy-resistant cancer.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In various embodiments, the administering comprises intravenous, intra-arterial, intra-tumoral, subcutaneous, topical, or intraperitoneal administration.

In some embodiments, the administering comprises local, regional, systemic, or continual administration.

In several embodiments, the method further comprises providing to a subject a second anti-cancer therapy. In some cases, the second anti-cancer therapy is surgery, chemotherapy, radiotherapy, hormonal therapy, toxin therapy, immunotherapy, and cryotherapy. The second anti-cancer therapy may be provided prior to administering the composition, the second anti-cancer therapy may be provided after administering the composition, and/or the second anti-cancer therapy may be provided contemporaneous with the composition.

In embodiments, the composition is administered daily (e.g., daily for 7 days, 2 weeks, 3 weeks, 4 weeks, one month, 6 weeks, 8 weeks, two months, 12 weeks, or 3 months).

In various embodiments, the composition is administered weekly (e.g., weekly for 2 weeks, 3 weeks, 4 weeks, 6 weeks, 8 weeks, 10 weeks, or 12 weeks).

In some embodiments, the composition is administered intermitantly.

In some embodiments, the compound or composition is administered in an amount sufficient to induce endoplasmic reticulum stress, in an amount sufficient to inhibit activity of LIPA, and/or in an amount sufficient to reduce protein synthesis.

The present disclosure also provides a composition for use in any herein-disclosed method.

In an aspect, the present disclosure provides a method for treating a cancer characterized by having increased activity of Lysosomal lipase A (LIPA) comprising administering a therapeutically-effective amount of a composition comprising the compound of Formula II:

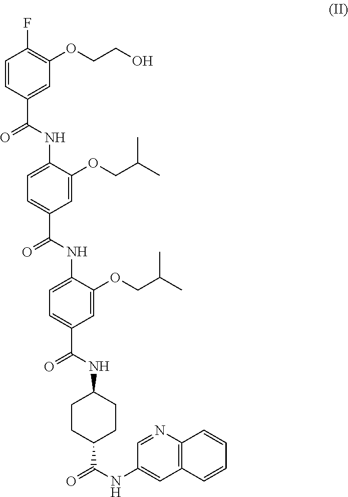 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

to a subject having a cancer with elevated LIPA expression.

In several embodiments, the compound is a pharmaceutically acceptable salt of Formula II.

In embodiments, the compound of Formula II inhibits LIPA activity when contacted to the LXXLL motif on LIPA.

In various embodiments, the compound or composition is administered in an amount sufficient to induce endoplasmic reticulum stress, in an amount sufficient to inhibit activity of LIPA, and/or in an amount sufficient to reduce protein synthesis.

In another aspect, the present disclosure provides a method for inhibiting activity of Lysosomal lipase A (LIPA) in a subject having a cancer comprising administering a therapeutically-effective amount of a composition comprising the compound of Formula II:

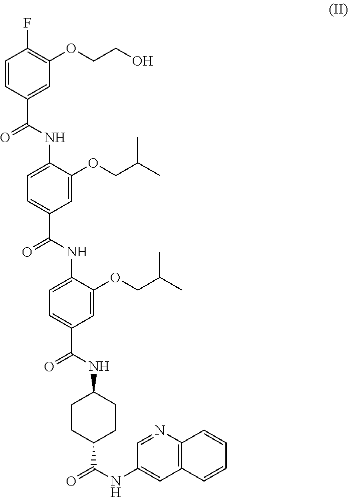 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

to the subject.

In some embodiments, the compound is a pharmaceutically acceptable salt of Formula II.

In several embodiments, the compound of Formula II inhibits LIPA activity when contacted to the LXXLL motif on LIPA.

In embodiments, the compound or composition is administered in an amount sufficient to induce endoplasmic reticulum stress, in an amount sufficient to inhibit activity of LIPA, and/or in an amount sufficient to reduce protein synthesis.

In a further aspect, the present disclosure provides a method for treating cancer in a subject in need thereof comprising testing a sample from the subject for the presence of elevated Lysosomal lipase A (LIPA) expression and administering a therapeutically-effective amount of a composition comprising the compound of Formula II:

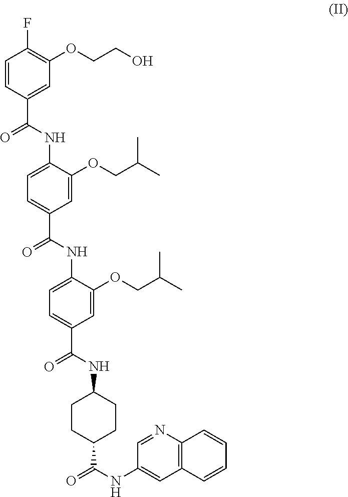 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

to a subject that provided a sample identified as having an elevated LIPA expression.

In various embodiments, the compound is a pharmaceutically acceptable salt of Formula II.

In some embodiments, the compound of Formula II inhibits LIPA activity when contacted to the LXXLL motif on LIPA.

In several embodiments, the compound or composition is administered in an amount sufficient to induce endoplasmic reticulum stress, in an amount sufficient to inhibit activity of LIPA, and/or in an amount sufficient to reduce protein synthesis.

An aspect of the present disclosure is a composition for use in a method of treating a cancer characterized by having increased activity of Lysosomal lipase A (LIPA), the method comprising administering a therapeutically-effective amount of a composition comprising a compound of Formula I:

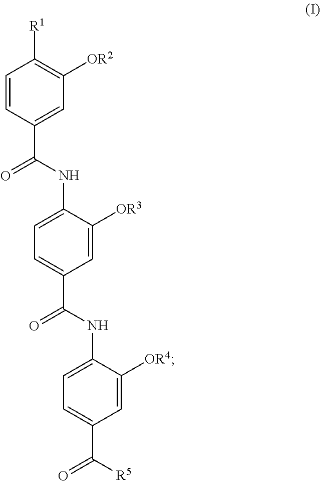

a subject having a cancer with elevated LIPA expression.

Another aspect of the present disclosure is a composition for use in a method of inhibiting activity of Lysosomal lipase A (LIPA) in a subject having a cancer, the method comprising administering a therapeutically-effective amount of a composition comprising a compound of Formula I:

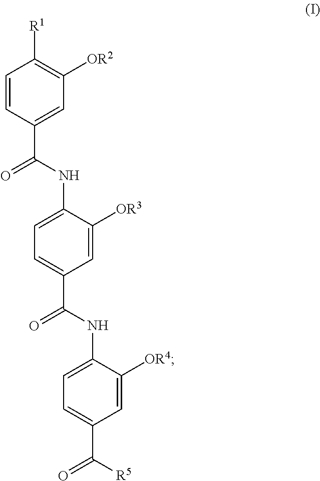

to the subject.

A further aspect of the present disclosure is a composition for use in a method of treating cancer in a subject in need thereof, the method comprising testing a sample from the subject for the presence of elevated Lysosomal lipase A (LIPA) expression and administering a therapeutically-effective amount of a composition comprising a compound of Formula I:

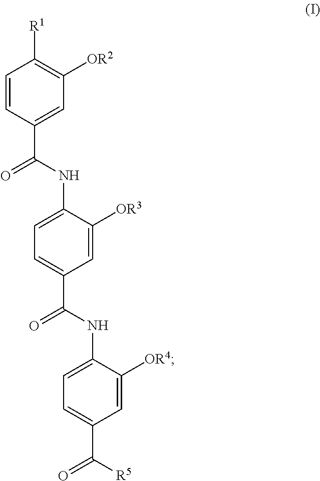

to a subject that provided a sample identified as having an elevated LIPA expression.

In embodiments, the compound of Formula I inhibits LIPA activity when contacted to the LXXLL motif on LIPA.

In various embodiments, the compound is a pharmaceutically acceptable salt of Formula I.

In some embodiments, R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein: R^{1a} is aralkyl_{(C<18)}, substituted aralkyl_{(C<18)}, or the side chain of a canonical amino acid; R², R³, and R⁴ are each independently alkyl_{(C<12)}, substituted alkyl_{(C<12)}, aralkyl_{(C<18)}, or substituted aralkyl_{(C<18)}; and R⁵ is —OR^{5a} or —NHR^{5b}, wherein: R^{5a} is alkyl_{(C<12)} or substituted alkyl_{(C<12)}; R^{5b} is cycloalkyl_{(C<12)}, aryl_{(C<12)} aralkyl_{(C<12)}, heteroaryl_{(C<12)}, heteroaralkyl_{(C<18)}, or a substituted version of any of these groups; or a group of the formula:

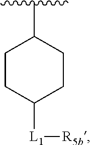

wherein; L is —CO₂— or —C(O)NR^{L}—, wherein: R^{L} hydrogen, alkyl_{(C<12)}, or substituted alkyl_{(C<12)}; R^{5b′} is aryl_{(C<12)}, aralkyl_{(C<18)}, heteroaryl_{(C<12)}, heteroaralkyl_{(C<18)}, or a substituted version of any of these groups.

In several embodiments, the compound comprises Formula II:

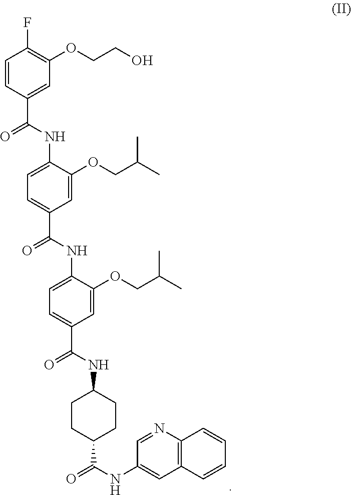 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

In embodiments, the compound comprises Formula III:

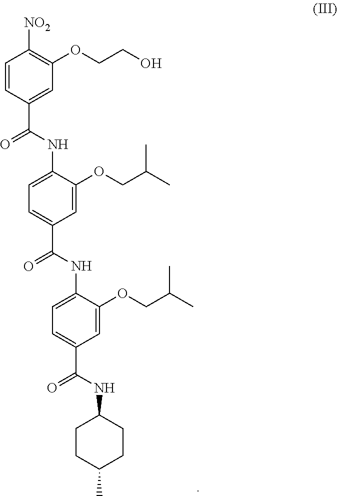 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

In some embodiments, the compound comprises Formula IV:

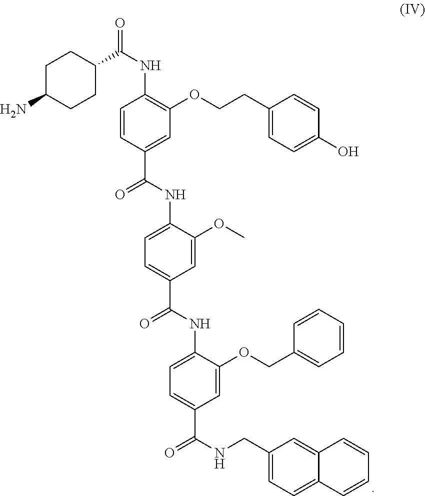 [SMILES: COc1cc(C(=O)Nc2ccc(C(=O)NCc3ccc4ccccc4c3)cc2OCc2ccccc2)ccc1NC(=O)c1ccc(NC(=O)[C@H]2CC[C@H](N)CC2)c(OCCc2ccc(O)cc2)c1]

In various embodiments, the method further comprises administering a therapeutically-effective amount of the composition.

In some embodiments, the compound is a pharmaceutically acceptable salt of Formula II, Formula III, or Formula IV.

In several embodiments, the compound of Formula II, Formula III, or Formula IV inhibits LIPA activity when contacted to the LXXLL motif on LIPA.

In embodiments, the compound or composition is administered in an amount sufficient to induce endoplasmic reticulum stress, in an amount sufficient to inhibit activity of LIPA, and/or in an amount sufficient to reduce protein synthesis.

Another aspect of the present disclosure is a method for determining if a subject is treatable (e.g., would get a therapeutic benefit) by a composition comprising the compound of Formula II:

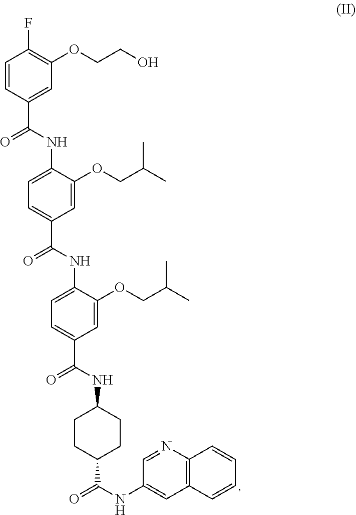 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

the method comprising testing a sample from the subject for the presence of elevated Lysosomal lipase A (LIPA) expression and when the sample indicates the presence of elevated LIPA or increased activity of LIPA, the subject is treatable by a composition comprising the compound of Formula II.

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In various embodiments, the method further comprises administering a therapeutically-effective amount of the composition.

In some embodiments, the compound is a pharmaceutically acceptable salt of Formula II.

In several embodiments, the compound of Formula II inhibits LIPA activity when contacted to the LXXLL motif on LIPA.

In embodiments, the compound or composition is administered in an amount sufficient to induce endoplasmic reticulum stress, in an amount sufficient to inhibit activity of LIPA, and/or in an amount sufficient to reduce protein synthesis.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

Any aspect or embodiment described herein can be combined with any other aspect or embodiment as disclosed herein.

### BRIEF DESCRIPTION OF THE DRAWINGS

For a more complete understanding of the features and advantages of the present disclosure, reference is now made to the detailed description of the disclosure along with the accompanying figures and in which:

FIGS. 1A-N: Identification of ERX-41. FIG. 1A: Schematic showing derivation of ERX-41 from ERX-11. FIG. 1B: Structures of ERX-11, ERX-11-9, ERX-11-16, ERX-11-30, ERX-41 and ERX-44. FIG. 1C: Heatmap showing ability of select oligobenzamides to block proliferation of ERα-positive breast cancer cells and TNBC cells in vitro. FIGS. 1D-G: MTT assays showing the effect of these selected oligobenzamides on cell viability of MCF-7 (FIG. 1D), ZR-75 (FIG. 1E), MDA-MB-231 (FIG. 1F), and SUM-159 (FIG. 1G) cells in vitro. Data shown are the means of ±SEM performed in triplicate wells. FIGS. 1H-I: Following establishment of MDA-MB-231 TNBC xenografts in mammary fat pad in vivo (100 mm³ size), daily administration of 10 mg/kg ERX-11-9, ERX-11-16, ERX11-30 or vehicle control (N=8 each group) was initiated. Body weights are shown for each set, including for ERX-11-9, ERX-11-16, ERX-11-30 administered subcutaneously (FIG. 1H) or orally (FIG. 1I). FIGS. 1J-K: Dose response curve of ERX-41 in multiple TNBC cell lines using WST-1 assays (FIG. 1J) and CellTiter-Glo assays (FIG. 1K). FIG. 1L: Time lapsed images of live cell imaging with SYTOX Green shows the effect of ERX-41 induced cell death in MDA-MB-231 at 0 h, 20 h and 30 h after treatment with either vehicle or ERX-41. FIGS. 1M-N: quantification of the number of dead cells over time seen with live cell imaging in MDA-MB-231 (FIG. 1M), HMEC cells (FIG. 1N). Data shown are the means of ±SEM performed in triplicate wells.

FIGS. 2A-P: ERX-41 is potent against TNBC in vivo. FIG. 2A. Following establishment of subcutaneous MDA-MB-231 xenograft tumors (100 mm³), 10 mg/kg single dose ERX-41 was administered either by oral or intraperitoneal route. The tumor was harvested 0, 0.5, 1.5, 3, 6 and 24 h after drug administration and the drug levels assayed by LC-MS/MS and graphed. FIGS. 2B-E: Following establishment of MDA-MB-231 xenografts in mammary fat pad in vivo (100 mm³ size), daily administration of 10 mg/kg ERX-41 or vehicle control (N=10 each group) was initiated. Tumor volumes were measured using digital calipers and graphed (FIG. 2B). Tumor weights (FIG. 2C), body weights (FIG. 2D) and extirpated tumors (FIG. 2E) at end of study are also shown. FIGS. 2F-H: Following establishment of D2A1 syngeneic xenografts in mammary fat pad in vivo (100 mm³ size), daily administration of 10 mg/kg ERX-41 or vehicle control (N=8 each group) was initiated. Tumor volumes were measured using digital calipers and graphed (FIG. 2F) along with tumor weights (FIG. 2G) and body weights (FIG. 211). FIGS. 2I-P: Effect of ERX-41 on the growth (FIG. 21, FIG. 2K, FIG. 2M, FIG. 2O) and tumor weights (FIG. 2J, FIG. 2L, FIG. 2N, FIG. 2P) of four distinct TNBC PDXs compared to vehicle.

FIGS. 3A-M: ERX-41 Induces ER stress in TNBC. FIGS. 3A-C: Volcano plots show the relative effect of 4 h treatment of 1 μM ERX-41 compared to vehicle in MDA-MB-231 (FIG. 3A) and BT-549 (FIG. 3B) TNBC cell lines. Heatmap of the top ER stress and UPR genes in these three TNBC cell lines after 2 h and 4 h treatment with 1 μM ERX-41 treatment (FIG. 3C). Fold change values in heatmap (D) were calculated by normalizing Fragments Per Kilobase of transcript per Million mapped reads (FPKM) values to the average FPKM values of vehicle condition. FIGS. 3D-F: Time course of the effect of 1 μM ERX-41 on the mRNA expression of canonical ER stress genes, sXBP1 (FIG. 3D), DDIT3 (FIG. 3E) and HSPA5 (FIG. 3F) in MDA-MB-231, BT-549 and HMEC cells. FIGS. 3G-H Transmission electron microscopy of MDA-MB-231 cells shows the effect of vehicle (FIG. 3G) and 1 μM ERX-41 treatment (FIG. 311) on subcellular structures at 4 h: ER is outlined with yellow arrowheads. FIGS. 3I-L: Airyscan imaging shows effect of 1 μM ERX-41 treatment on SUM-159 cells stably expressing the ER membrane marker mCherry-RAMP4 without treatment (FIG. 3I) and 2 h (FIG. 3J) after treatment: inset detail is shown on the right panel. Representative graph shows the distribution of normalized tubule intensity (white lines in inset panel of FIG. 3I and FIG. 3J) from both vehicle and ERX-41 treated samples (FIG. 3K). Dilated ER is shown by two peaks, with signal between the peaks, indicating a continuous dilated ER tubule. Histogram comparing the distribution of ER tubule widths (in nm) in the untreated and treated samples is shown (FIG. 3L). FIG. 3M. Western blotting shows the time course of the effect of 1 μM ERX-41 or ERX-11 on the expression of UPR proteins in SUM-159 cells.

FIGS. 4A-L: The molecular target of ERX-41 in TNBC is LIPA. FIG. 4A: Visualization of a CRISPR/Cas9 screen in MDA-MB-231 cells shows the genes associated with resistance to ERX-41. The top 5 genes are highlighted. FIG. 4B: Knockout clones of LIPA, SLCSA3, TMEM208, SOAT1 and ARID1A were generated in MDA-MB-231 cells and evaluated for response to ERX-41 using dose-response curves using CellTiter-Glo assays in vitro. Each gene was knocked out by two different sgRNAs. FIGS. 4C-D: Effect of knockout of LIPA in SUM-159 (FIG. 4C) and MDA-MB436 (FIG. 4D) on dose response curve to ERX-41 using CellTiter-Glo assays in vitro. FIGS. 4E-F: Dose response curves of SUM-159 parental cells and clones with LIPA KO to paclitaxel (FIG. 4E) and thapsigargin (FIG. 4F). FIGS. 4G-H: Live cell imaging studies with SYTOX Green to examine ability of 1 μM ERX-41 to induce cell death in parental SUM-159 and SUM-159 clones with LIPA KO, with quantitation (FIG. 4G) and time-lapsed photomicrographs at 0 h and 30 h (FIG. 4H). FIGS. 4I-L: SCID mice were implanted with either parental SUM-159 or SUM-159 clones with LIPA KO (n=6, each group) and tumors were allowed to establish (150 mm³). Following daily intraperitoneal administration of 10 mg/kg ERX-41, tumor sizes were measured and graphed (FIG. 4I), along with mice body weights (FIG. 4J). At the time of sacrifice, xenograft tumors from the untreated and treated mice were harvested and processed for IHC with Ki67. Representative immunohistochemical staining is shown (FIG. 4K) and proliferative indices for each tumor were quantitated and graphed (FIG. 4L).

FIGS. 5A-L: LIPA is the target of ERX-41. FIGS. 5A-D Following treatment with either vehicle or 1 μM ERX-41 for 4 h in parental SUM-159 and SUM-159 clones with LIPA KO, RNA was isolated and subject to unbiased RNA sequencing. Principal component analyses of the sequencing data (FIG. 5A) volcano plots in parental (FIG. 5B) and LIPA KO cells (FIG. 5C) and heatmap of canonical genes involved in ER stress and UPR response (FIG. 5D) are shown. -Fold change values in heatmap (D) were calculated by normalizing FPKM values to the average FPKM values of WT vehicle condition. FIGS. 5E-F: RT-qPCR evaluation of expression of sXBP1 (FIG. 5E) and DDIT3 (FIG. 5F) in parental SUM-159 cells and SUM-159 cells with LIPA KO. FIGS. 5G-J: Live cell confocal microscopy shows the serial effect of 1 μM ERX-41 treatment on the morphology of SUM-159 cells stably expressing the ER membrane marker mCherry-RAMP4 at 0, 2 and 4 h (FIG. 5G). The panels show serial magnification of the same field of view, with field view (top panel), single cell view (middle panel), and detail (lower panel). The same experiment was repeated in SUM-159 cells stably expressing mCherry-RAMP4 and with LIPA KO (FIG. 5H). The histogram shows the effect of 1 μM ERX-41 on the ER tubule width in the parental (FIG. 5I) and LIPA KO cells (FIG. 5J) (n=25 each condition). FIG. 5K: Western blot shows the effect of 1 μM ERX-41 treatment on the expression of LAL and ER stress proteins p-eIF2α and PERK in parental SUM-159 and SUM-159 cells with LIPA KO. FIG. 5L: Time course of effect of 1 μM ERX-41 on global de novo protein synthesis in parental SUM-159 and SUM-159 cells with LIPA KO shown by western blots for puromycin labeled nascent proteins. Effect in HMEC cells is also shown for comparison.

FIGS. 6A-K: LIPA is a viable molecular target in TNBC and other cancers. FIGS. 6A-B: Samples from 51 TNBC patients and 20 normal breast tissue were evaluated for LAL expression using TNBC tissue microarray, with representative images (FIG. 6A) and quantitation (FIG. 6B). FIG. 6C: Western blots show expression of LAL protein in 4 matched tumor (T) and adjacent normal (N) tissues. FIG. 6D: Quantitation of expression of LAL proteins from 10 matched tumor (T) and adjacent normal (N) tissues. FIG. 6E: Kaplan-Meier curves show the correlation between expression levels of LAL with survival outcomes using a TCGA dataset with numerical tabulation below. FIGS. 6F-H: Following surgical extirpation, primary TNBC tumors were cultured ex vivo either with vehicle or 1 μM ERX-41, as shown by the schematic (FIG. 6F). The effect on proliferation index (Ki67) (FIG. 6G) and apoptosis marker (cleaved caspase 3) (FIG. 6H) is graphed. Representative immunohistochemical images of the effect of ERX-41 on Ki67, cleaved caspase 3 and UPR proteins shown (FIG. 6I). FIG. 6J: Following establishment of ERα+ WHIM-20 breast cancer PDX in mammary fat pad in NSG mice (100 mm³ size), daily administration of 10 mg/kg ERX-41 or vehicle control (N=6 tumors per group) was initiated. Tumor volumes were measured using digital calipers and graphed. Tumor weights and extirpated tumors at end of study are shown. FIG. 6K: Following establishment of subcutaneous ovarian cancer PDX (OCa-PDX-38) tumors in NSG mice (100 mm³ size), daily administration of 10 mg/kg ERX-41 or vehicle control (N=6 tumors per group) was initiated. Tumor volumes were measured using digital calipers and graphed. Tumor weights and extirpated tumors at end of study are shown.

FIGS. 7A-U: ERX-41 binds LAL but does not affect its lipase activity. FIGS. 7A-C: Cellular thermal shift assays were performed to evaluate the heat stability of LAL or vinculin protein in SUM-159 cells (FIG. 7A) and the relative levels of LAL (FIG. 7B) and vinculin (FIG. 7C) were quantitated and graphed. FIGS. 7D-H: Predicted binding mode of ERX-41 (colored in green) on the human lysosomal acid lipase (LAL, PDB code: 6V7N, LXXLL domain colored in orange). FIG. 7I: PDB structure of LAL protein showing relative positions of the catalytic domain (with H274Y MT) and the LXXLL domain (L242P MT). FIG. 7J: Cellular lysates from described SUM-159 cells were incubated with biotinylated ERX-11 or biotinylated ERX-41 and then subject to a streptavidin column. Eluants were then evaluated for LAL pulldown by western blotting. FIG. 7K: Dose response curve to increasing concentrations of ERX-41 in described SUM-159 cells, as measured by CTG studies. FIG. 7L: Evaluation of basal lipase activity in parental SUM-159 (WT), SUM-159 cells with LIPA KO (KO), SUM-159 cells with LIPA KO and with reconstitution of either WT LIPA (KO+WT), H274Y MT-LIPA (KO+H274Y), ΔLXXLL MT-LIPA (KO+ΔLXXLL) or L242P MT-LIPA (KO+L242P). Protein expression of LAL and control vinculin in these cells are shown below. These results are tabulated for review (FIG. 7M). FIGS. 7N-P: Effect of vehicle or 1 μM ERX-41 on induction of UPR genes at the protein level (FIG. 7N) or RNA level (FIGS. 7O-P) in SUM-159 cells with LIPA KO and reconstitution with various LIPA constructs. FIGS. 7Q-U: Effect of ER localization: Recombinant cDNAs with full length LIPA (WT), lacking the signal peptide (WT-ASP) or with the ER-retention peptide (WT-KDEL) were synthesized (FIG. 7Q). These constructs were then stably transfected in SUM-159 cells with LIPA KO and clones selected. The sensitivity of the expressed recombinant LAL protein in these cells to Endo H or PNGase F cleavage is shown. Effect of vehicle or 1 μM ERX-41 on induction of UPR genes at the protein level in SUM-159 cells with LIPA KO and reconstitution with various LIPA constructs (FIG. 7S). Dose response curves to increasing concentrations of ERX-41 in these SUM-159 cells were performed by CellTiterGlo and graphed (FIG. 7T). These results are also summarized in a table (FIG. 7U).

FIGS. 8A-P: FIG. 8A: Structure of recombinant LIPA fused to TurboID under the control of a doxycycline-inducible promote (TRE). FIG. 8B: CellTiter-Glo studies showing the effect of ERX-41 on the growth of clones of SUM-159 cells with LIPA KO stably transfected with the recombinant LIPA TurboID construct without and with doxycycline induction. Figure SC: Schematic shows that LAL interacting proteins (blue) are biotinylated while the LAL non-interacting proteins (grey boxes) are not biotinylated. The biotinylated proteins are then isolated by streptavidin binding and identified by unbiased mass-spectrometric analyses. FIG. 8D: Venn diagram shows overlap between the two replicates of LAL binders. FIGS. 8E-F: Gene ontogeny analysis shows both the biological processes (FIG. 8E) and cellular component (FIG. 8F) of the top 54 LAL binders. FIG. 8G: Schematic of global data-independent acquisition mass spectrometry (DIA-MS). FIG. 8H: Principal component analyses show the effect of ERX-41 on the expression of a number of proteins. FIG. 8I: Summary of DIA analyses shows the expression of a number of proteins significantly affected by ERX-41. FIGS. 8J-K: Gene ontogeny analysis shows both the biological processes (FIG. 8J) and cellular component (FIG. 8K) of the top 153 ERX-41-downregulated proteins. FIG. 8L: Venn diagram shows overlap between ERX-41 down-regulated proteins and LAL binders, with the protein list displayed. FIGS. 8M-N: Gene ontology analysis shows both the biological processes (FIG. 8M) and cellular component (FIG. 8N) of the 17 ERX-41-downregulated and LAL-binding proteins binders. Western blot shows the effect of ERX-41 in SUM-159 cells (WT) and SUM-159 cells with IPA KO (KO) on the expression of LAL, ER stress markers and number of ER resident proteins identified in FIG. 8L. FIG. 8P: Model shows that ERX-41 binds to the LXXLL domain of LAL protein and induces ER stress resulting in cell death.

FIGS. 9A-G: Over 200 analogues of ERX-11 were synthesized and tested for antiproliferative activity using MTT assays in ZR-75, MCF-7 and MCF-7/Tamoxifen resistant ERα+ cell lines and MDA-MB-231 and SUM-159 TNBC cells. The heatmap of the antiproliferative response of these cell lines to selected compounds is shown (FIG. 9A). The IC₅₀ in nM as measured by MTT assays for four oligobenzamides in these cell lines is tabulated (FIG. 9B). The IC₅₀ in nM for ERX-41 in multiple TNBC cells (along with their molecular subtype) is tabulated for the WST-1 assay (FIG. 9C) and the CTG assay (FIG. 9D). Time lapsed images of live cell imaging with SYTOX Green shows the effect of ERX-41 induced cell death in HMEC cells at 0 h, 20 h and 30 h after treatment (FIG. 9E). FIGS. 9F-G: The ¹H- and ¹³C-NMR spectra of the hit compound ERX-41 are shown (FIG. 9F and FIG. 9G, respectively).

FIGS. 10A-F: After 30 days of oral ERX-41 (10 mg/kg/day) or vehicle treatment, histologic architecture of multiple organs in either nude mice (with MDA-MB-231 xenograft tumors) or syngeneic mice (with D2A1 xenograft tumors) were evaluated using H&E staining (FIG. 10A). The uterus in these mice were also evaluated for changes in their proliferation index (ki67 staining (FIG. 10B, with quantitation on the right side)), or endometrial cell height (FIG. 10C, with quantitation), or ERα expression (FIG. 10D). FIGS. 10E-F: Evaluation of the effect of 1 week of oral ERX-41 (10 mg/kg/day) on the distribution of plasma cells in the bone marrow by flow cytometry (FIG. 10E) and antibody-secreting cells (ASCs) that produces IgM or IgG in the bone marrow using ELISPOT analysis (FIG. 10F) with quantitation.

FIGS. 11A-K: FIGS. 11A-C: GSEA analysis of the RNA-sequencing data evaluating the treatment-enriched gene sets in three TNBC cell lines, MDA-MB-231 (FIG. 11A), BT-549 (FIG. 11B) and SUM-159 (FIG. 11C) following 4 h exposure to 1 μM ERX-41. The top 10 gene sets (total ˜15k C5 ontology gene sets) are shown for each cell line. ER− related gene sets are highlighted in blue. For all shown gene sets, the adjusted P value is <0.001 for all gene sets. Gene ontology analyses show the correlation between ERX-41 treatment and induction of the GO intrinsic apoptotic signaling pathway in response to ER stress in each of these three TNBC cell lines (FIG. 11D). FIGS. 11E-G: Western blotting shows the time course of the effect of 1 μM ERX-41 on the expression of UPR proteins in MDA-MB-231 (FIG. 11E), BT-549 (FIG. 11F) and HMEC cells (FIG. 11G). FIGS. 11H-J: Immunohistochemical evaluation of the effect of a single dose 10 mg/kg oral ERX-41 on the expression of ER stress markers in MDA-MB-231 TNBC tumor xenografts at 24 h after treatment (FIG. 11H), with quantitation of p-eIF2α (FIG. 11I) and p-PERK (FIG. 11J) in tumor tissue. FIG. 11K: Time course of effect of 1 μM ERX-41 on global de novo protein synthesis in two TNBC (MDA-MB-231 and BT-549) and HMEC cells shown by western blots for puromycin labeled nascent proteins.

FIGS. 12A-K: FIGS. 12A-B: Schematic representation of LanthaScreen TR-FRET ER Alpha Coactivator assay (FIG. 12A). W-anti-GST antibody indirectly labels ERα by binding to the GST tag. Binding of the agonist to ERα causes a conformational change, leading to an increase in the affinity of ERα for a coactivator LXXLL peptide. The close proximity of the fluorescently labeled coactivator peptide to the terbium-labeled antibody results in an increase in the TR-FRET signal. Representative data from TR-FRET ERα Coactivator assay run in antagonist mode. Results after 2-hour incubation for ERX-41, Fulvestrant (ICI) and a SERD (GDC-0810) are graphed and the kd values tabulated below (FIG. 12B). Curves were generated using a sigmoidal dose response equation in GraphPad Prism software. FIG. 12C: Correlation between two biologic replicates of the CRISPR screen in MDA-MB-231 cells is graphed with the overlap depicted using Venn diagram. The top 5 genes in both replicates are highlighted. FIGS. 12D-H: Western blotting shows the identification of clones of MDA-MB-231 (outlined in red) showing protein level knockout of expression of TMEM208 (FIG. 12D), ARID1A (FIG. 12E), SOAT1 (FIG. 12F) SLC5A3 (FIG. 12G), and LIPA (FIG. 12H). FIGS. 12I-K: Effect of four individual CRISPR guide RNAs on the responsiveness of MDA-MB-231 cells to vehicle or ERX-41 treatment, as shown for LIPA (FIG. 12I), PERK (FIG. 12J) and IRE1α (FIG. 12K).

FIGS. 13A-K: FIGS. 13A-C: Dose response curve of the effect of ERX-41 on the proliferation of parental MDA-MB-436 cells and two MDA-MB-436 clones with LIPA KO by CellTiter-Glo studies (FIG. 13A). RT-qPCR evaluation of the effect of 1 μM ERX-41 on expression of sXBP1 (FIG. 13B) and DDIT3 (FIG. 13C) in parental MDA-MB-436 cells and MDA-MB-436 clones with LIPA KO is graphed. FIG. 13D: Effect of ERX-41 on the proliferation of parental SUM-159 cells and SUM-159 cells with LIPA KO stably expressing the ER membrane marker mCherry-RAMP4 as measured by CellTiter-Glo. FIG. 13E: Western blot shows effect of 1 μM ERX-41 or thapsigargin or tunicamycin treatment on expression of UPR proteins in parental SUM-159 and SUM-159 cells with LIPA KO. FIGS. 13F-I: Immunofluorescence shows colocalization of myc-tagged LAL protein with either ER (ER tracker) or lysosomal markers (LIMP2) in SUM-159 cells with overexpressed LAL-myc (FIG. 13F) and in parental SUM-159 cells without LAL-myc (FIG. 13I). Colocalization between expression parameters in the LAL-myc expressing cells are graphically correlated with quadrant quantification (FIG. 13G) and weighted colocalization factor calculations tabulated (FIG. 13H). FIG. 13J: SUM-159-KO+WT cell pellets were suspended in PBS+0.5% BSA and subjected to sonication and sequential centrifugation (2000 g for 12 min, 15000 g for 60 min). Pellet after 2000 g centrifugation is nuclear fraction (enriched for histone H3). Supernatant after 15000 g centrifugation is ER fraction (enriched for calreticulin). Pellet after 15000 g centrifugation is lysosome fraction (enriched for LAMP2). These fractions were electrophoresed and evaluated for expression of each subcellular fraction marker and LAL using two antibodies. K: Sensitivity of glycosylated LAL to endoglycosidase H (endo H) or Peptide-N-Glycosidase F (PNGase F) cleavage. Lysates from SUM-159 and MDA-MB-231 cells were subject to Endo H or PNGase digestion and then LAL expression evaluated. Glycosylated LAL sensitive to endoH or PNGase F cleavage is highly suggestive of ER subcellular localization (FIG. 13K).

FIGS. 14A-N: FIG. 14A: Demographics of the patients from whom the TNBC TMA was constructed are tabulated. FIG. 14B: Demographics of the patients from whom the primary tumor explants were obtained from are tabulated. FIG. 14C: Sensitivity of glycosylated LAL to endoglycosidase H (endo H) or Peptide-N-Glycosidase F (PNGase F) cleavage. Lysates TNBC tumor or adjacent normal tissues from the same patient were subject to Endo H or PNGase digestion and then LAL and vinculin expression was evaluated. FIG. 14D: Tissues from mice were harvested after daily administration of 10 mg/kg ERX-41 for 1 week and then evaluated for expression of LAL protein using immunohistochemistry with the Santa Cruz monoclonal antibody that recognizes both mouse and human LAL protein. Inset shows detail of the highlighted area. FIG. 14E: Dose response curves of parental and LIPA KO clones of ERα+ MCF-7 cells show the effect of increasing concentrations of ERX-41 at 48 h after treatment. FIG. 14F: Evaluation of the effect of 1 μM ERX-41 on the induction of p-PERK (up shifted band) in parental and LIPA KO clones of ERα+ MCF-7 cells. FIGS. 14G-I: CTG assays showing the dose response curves for the effect of ERX-41 on cell viability of a number of glioblastoma (FIG. 14G), pancreatic (FIG. 14H) and ovarian (FIG. 14I) cancer cell lines in vitro. FIGS. 14J-N: ES2/GFP/LUC cells were injected i.p. and tumors established. Mice (n=3) were treated with vehicle or ERX-41 (10 mg/kg/day/i.p). Tumor volume (FIG. 14J), tumor picture (FIG. 14K), tumor weight (FIG. 14L), number of tumor nodules (FIG. 14M) and ascites volume (FIG. 14N) is shown. *P<0.05. **P<0.01; ****P<0.0001.

FIGS. 15A-J: FIG. 15A: Schematic structure of LAL is depicted with its 21 as N-terminal signal peptide, core domain (residues 1-183 and 309-378), and cap domain (residues 184-308). The putative propeptide of the core domain (residues 1-56) is indicated. In the cap domain, there is a flexible lid consisting of residues 212 to 251. Residues 238-244 include the LXXLL motif and the L242P point mutation within this motif are highlighted. The point mutation H274Y in the cap that renders LAL lipase dead is shown. The depiction is not drawn to scale. FIG. 15B: Evaluation of the effect of ERX-41 and Lalistat 2 (known LAL lipase inhibitor) on lipase activity of parental SUM-159 (WT) and SUM-159 cells with LIPA KO. FIG. 15C: Crystal violet staining of parental SUM-159 (WT), SUM-159 cells with LIPA KO (KO), SUM-159 cells with LIPA KO and with reconstitution of either WT LIPA (KO+WT), or ΔLXXLL MT-LIPA (KO+ΔLXXLL) after 3 days' treatment with vehicle or 1 μM ERX-41. D-F: Effect of LXXLL mutations: SUM-159 cells with LIPA KO reconstituted with recombinant LIPA with described LXXLL mutations. Dose response curve to increasing concentrations of ERX-41 in these SUM-159 cells were performed by cellTiterGlo (FIG. 15D). Effect of vehicle or 1 μM ERX-41 on induction of UPR gene at the protein level in these SUM-159 cells is evaluated by western blotting (FIG. 15E). These results are tabulated (FIG. 15F). FIGS. 15G-J: Effect of mouse LIPA: SUM-159 cells with LIPA KO reconstituted with recombinant LIPA with either the mouse LXXLL sequence or complete mouse LIPA cDNA (mLIPA). Dose response curves to increasing concentrations of ERX-41 in these SUM-159 cells were performed by CellTiterGlo and graphed (FIG. 15G). The sensitivity of the expressed recombinant LAL protein in these cells to Endo H or PNGase F cleavage is shown (FIG. 15H). Effect of vehicle or 1 μM ERX-41 on induction of UPR gene at the protein level in these cells is shown by western blotting (FIG. 15I). These results are tabulated (FIG. 15J).

FIGS. 16A-D: FIG. 16A: Effect of vehicle or 1 μM ERX-41 on induction of UPR gene at the protein level in SUM-159 LIPA KO cells with or without LIPA-TurboID expression is evaluated by western blotting. FIG. 16B: Western blotting shows the induction of biotinylated proteins in two different clones expressing pCW-LIPA-TurboID after 2 days' treatment of doxycycline. FIG. 16C: The gene names of proteins down regulated by ERX-41. FIG. 16D: The heatmap of differential expressed proteins (P<0.01 and fold change > or <1.5) identified by DIA-MS.

FIG. 17 shows the effects of ERX-315 on various cancer cell lines.

FIGS. 18A-D show that oral administration of TK315 (ERX-315) decreased the growth and tumor weight of BC xenografts genetically engineered by CRISPR to express the Y537S ERα mutant in the ZR75 (FIGS. 18A-B) and MCF7 cells (FIGS. 18C-D). No change in body weight was noted.

FIGS. 19A-B: MTT assays showing the effect of TK-342 (Formula IV) on cell viability of SUM-159 cells (WT) and SUM-159 cells with LIPA KO and LIPA OE, as well as cell viability of SUM-159 cells (WT) and SUM-159 cells with SOAT1. Data shown are the means of ±SEM performed in triplicate wells.

### DETAILED DESCRIPTION

The present disclosure provides oligo-benzamide peptidomimetic compounds for use in the treatment and/or prevention of cancer, particularly breast cancer, including triple-negative breast cancer (TNBC). These small molecules include α-helix mimetics that represent helical segments in the target molecules. The oligo-benzamide peptidomimetic compounds modulate protein-protein, protein-peptide, or protein-drug interaction to exert a variety of physiological consequences. The oligo-benzamide peptidomimetic compounds also cause significant endoplasmic reticulum stress in cancer cells and may effectively shut down de novo protein synthesis, leading to cell death.

Interaction between proteins regulate most biological processes and occur through specific motifs. LXXLL motifs, where L is leucine and X is any amino acid, were first identified as critical for the interaction between nuclear receptors (NR) and protein cofactors.^{1,2} However, LXXLL motifs are not only restricted to NR signaling but are widely noted to be involved in many protein-protein interactions associated with different aspects of cellular regulation.³ Importantly, specificity of interactions is determined by the composition of residues that flank the LXXLL motifs, the alpha helical propensity and availability of partnering proteins for interaction.^{4,5} Prior studies with peptides targeting selective LXXLL motifs using in vitro models have indicated the potential specificity and therapeutic utility of selective LXXLL targeting agents. However, further research into this class of agents is therefore warranted.

Peptidomimetics (also known as peptide mimetics) are small organic molecules that do not possess the peptide backbone structure, however, still retain a capability to interact with the same target protein by arranging essential functional groups (i.e., pharmacophores) in a required three-dimensional pattern complimentary to a binding pocket in the protein. Since peptides and proteins adopt and utilize secondary structures (e.g., α-helix, β-sheet, and reverse turns) to make their global shapes and to recognize their binding partners, rational design of secondary structure mimetics is an important strategy in developing small molecule modulators for protein complex formation, compared to conventional high-throughput screening of a chemical library.

The inventors previously showed the utility of oligobenzamide-based peptidomimetics to target the LXXLL motif in specific proteins. They identified oligobenzamides, D2 and ERX-11, that could bind to the androgen receptor (AR)⁶ and estrogen receptor alpha (Erα)⁷, respectively, and blocked their interactions with a number of critical protein cofactors through the LXXLL domain. Importantly, ERX-11 was shown to block ERα signaling and ERα-driven proliferation with an IC₅₀ of 200-500 nM in both therapy-sensitive and therapy-resistant ERα-positive breast cancers.

During further development of ERX-11 for functional activity, the inventors synthesized >200 oligobenzamides. They discovered that relatively “minor” modifications resulted in significant changes in specificity and activity of the analogs.⁶ As part of these tests, they included TNBC cells (which lack expression of ERα), for which D2 and ERX-11 did not show anti-tumor activity. The inventors were intrigued by the serendipitous finding that some oligobenzamide analogs were highly active in inhibiting the growth of TNBC cells. Since LXXLL motifs are also involved in non-NR-driven cellular processes, this data indicated that these oligobenzamides may target an alternative molecular driver in TNBC, which currently lacks rational targeted therapy.

Variants, analogs, and/or derivatives of ERX-11 include the oligo-benzamide peptidomimetic compounds of the present disclosure, which relate to Formula I:

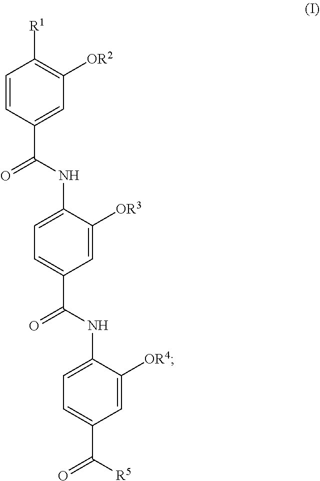

or a pharmaceutically acceptable salt thereof, wherein:
R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein:
   R^{1a} is aralkyl_{(C<18)}, substituted aralkyl_{(c<18)}, or the side chain of a canonical amino acid;
R², R³, and R⁴ are each independently alkyl_{(C<12)}, substituted alkyl_{(C<12)}, aralkyl_{(C<18)}, or substituted aralkyl_{(C<18)}; and
R⁵ is —OR^{5a} or —NHR^{5b}, wherein:
   R^{5a} is alkyl_{(C<12)} or substituted alkyl_{(C<12)};
   R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or a group of the formula:

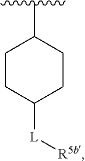

wherein;
L is —CO₂— or —C(O)NR^{L}—, wherein:
R^{L} hydrogen, alkyl_{(C<12)}, or substituted alkyl_{(C<12)};
R^{5b}, is aryl_{(C<12)}, aralkyl_{(C<18)}, heteroaryl_{(C<12)}, heteroaralkyl_{(C<18)}, or a substituted version of any of these groups.

An ERX-11 variant, analog, and/or derivative, ERX-315, demonstrated potent activity against ovarian cancer cells and breast cancer cells, respectively, with ICso values of from 10-50 nM. It has been shown, (e.g., in WO2020117715A1, the contents of which is incorporated herein by reference in its entirety to be an extremely potent compound that inhibits tumor growth and kills breast and ovarian cancer cells and as such, is a superb therapeutic candidate for such diseases.

Thus, an illustrative oligobenzamide-based peptidomimetic of the present disclosure is modified with a quinoline group at the southern terminus of the compound. This compound, of Formula II and referred here as ERX-315 or TK315, is described herein as having one or more preferential properties relative to those known in the art, such as improved efficacy.

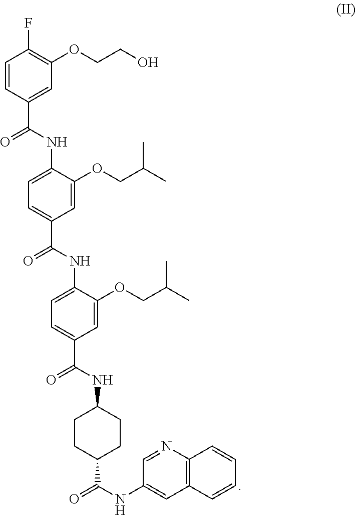 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

Another of the ERX-11 variants, analogs, and/or derivatives, ERX-41, demonstrated potent activity against multiple forms of TNBC, in vitro, ex vivo and in vivo. The inventors found that ERX-41 dramatically induces endoplasmic reticulum (ER) stress and uncompensated unfolded protein response (UPR) in TNBC, leading to cell death. In contrast, ERX-41 was non-toxic against normal mammary epithelial cells. Using a CRISPR knockout screen, the inventors discovered LAL, protein product of gene LIPA, as the molecular target of ERX-41 in TNBC and identified a previously uncharacterized function of IPA in the ER as critical for ERX-41 activity. ERX-41 has demonstrated activity against other tumors including pancreatic, glioblastoma, ovarian and ERα+ breast cancers. Taken together, these data reveal a novel therapeutic agent (ERX-41) with activity against a novel molecular target (LAL) in multiple cancers.

Accordingly, another illustrative oligobenzamide-based peptidomimetic is modified with a cyclohexylamide group at the southern terminus of the compound. This compound, of Formula III and referred here as ERX-41 or TK41, is described herein as having one or more preferential properties relative to those known in the art, such as improved efficacy.

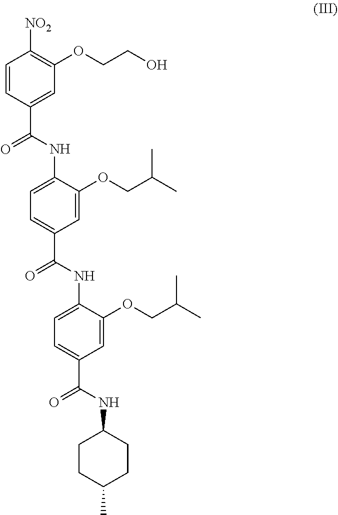 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

The present disclosure provides synthetic molecules which present the essential functionalities of corresponding peptide ligands in the proper three-dimensional orientation that enables specific protein interactions, leading to either stimulation or inhibition of protein-mediated functions. To mimic α-helices, the present disclosure provides an oligo-benzamide scaffold that is rigid in structure and place and orient substituents as an α-helix does. Substitution on the rigid tris-benzamide, for instance, allowed easy placement of three functional groups (R₂₋₄) corresponding to the side chains of amino acids found at the i, i+4, and i+7 positions of an ideal α-helix. Furthermore, the present inventors have developed a facile synthetic route to prepare a number of tris-benzamides to represent α-helical segments of target proteins. U.S. Patent Publication 2009/0012141, incorporated herein by reference, discloses a variety of oligo-benzamide compounds and methods of synthesis therefor.

More specifically, the present disclosure provides an oligo-benzamide peptidomimetic compound as illustrated includes 2 or 3 optionally substituted benzamides—so called “bis” and “tris” benzamides. In addition, linkages between the optionally substituted benzamides may be varied as necessary including ester, thioester, thioamide, trans-ethylene, ethyl, methyloxy, methylamino, hydroxyethyl, carbamate, urea, imide, hydrozido, aminoxy, or other linkages known to the skilled artisan. And, the oligo-benzamide peptidomimetic compound may be attached to amino acids, oligopeptides, optionally substituted alkyl, or other structures known to the skilled artisan.

The substitution on the substituted benzamide is generally on a benzene ring and may be on the 2, 3, 4, 5, or 6 position of each of the benzene rings. The substitutions may be at the same position on each of the benzamide rings but may also be at different positions on each of the benzene rings. For example, the substitution is connected to the benzamide ring by a chemical linkage including ether, thioether, amine, amide, carbamate, urea, and carbon-carbon (single-, double-, and triple-) bonds, and the substitution comprises optionally substituted alkyl groups, lower alkyl groups, alkoxy groups, alkoxyalkyl groups, hydroxy groups, hydroxyalkyl groups, alkenyl groups, amino groups, imino groups, nitrate groups, alkylamino groups, nitroso groups, aryl groups, biaryl groups, bridged aryl groups, fused aryl groups, alkylaryl groups, arylalkyl groups, arylalkoxy groups, arylalkylamino groups, cycloalkyl groups, bridged cycloalkyl groups, cycloalkoxy groups, cycloalkyl-alkyl groups, arylthio groups, alkylthio groups, alkylsulfinyl groups, alkylsulfonyl groups, arylsulfonyl groups, arylsulfinyl groups, caboxamido groups, carbamoyl groups, carboxyl groups, carbonyl groups, alkoxycarbonyl groups, halogen groups, haloalkyl groups, haloalkoxy groups, heteroayl, heterocyclic ring, arylheterocyclic ring, heterocyclic compounds, amido, imido, guanidino, hydrazido, aminoxy, alkoxyamino, alkylamido, carboxylic ester groups, thioethers groups, carboxylic acids, phosphoryl groups or combination thereof.

The present disclosure also provides an oligo-benzamide peptidomimetic compound that includes at least two optionally substituted benzamides, with each of the substituted benzamides having one substitution on a benzene ring. The substitutions are individually attached to the benzene rings of the oligo-benzamide peptidomimetic compound by a chemical linkage including ether, thioether, amine, amide, carbamate, urea, and carbon-carbon (single-, double-, and triple-) bonds. The substitutions generally include optionally substituted alkyl groups, lower alkyl groups, alkoxy groups, alkoxyalkyl groups, hydroxy groups, hydroxyalkyl groups, alkenyl groups, amino groups, imino groups, nitrate groups, alkylamino groups, nitroso groups, aryl groups, biaryl groups, bridged aryl groups, fused aryl groups, alkylaryl groups, arylalkyl groups, arylalkoxy groups, arylalkylamino groups, cycloalkyl groups, bridged cycloalkyl groups, cycloalkoxy groups, cycloalkyl-alkyl groups, arylthio groups, alkylthio groups, alkylsulfinyl groups, alkylsulfonyl groups, arylsulfonyl groups, arylsulfinyl groups, caboxamido groups, carbamoyl groups, carboxyl groups, carbonyl groups, alkoxycarbonyl groups, halogen groups, haloalkyl groups, haloalkoxy groups, heteroayl, heterocyclic ring, arylheterocyclic ring, heterocyclic compounds, amido, imido, guanidino, hydrazido, aminoxy, alkoxyamino, alkylamido, carboxylic ester groups, thioethers groups, carboxylic acids, phosphoryl groups or combination thereof.

An aspect of the present disclosure is a composition for use in a method of treating a cancer characterized by having increased activity of Lysosomal lipase A (LIPA), the method comprising administering a therapeutically-effective amount of a composition comprising a compound of Formula I:

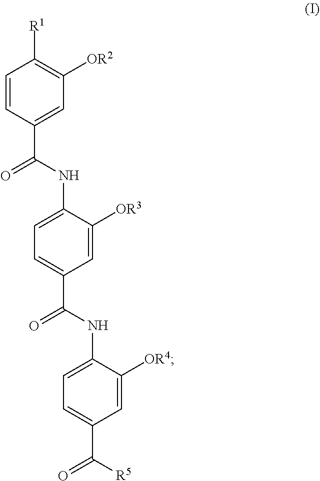

to a subject having a cancer with elevated LIPA expression.

Another aspect of the present disclosure is a composition for use in a method of inhibiting activity of Lysosomal lipase A (LIPA) in a subject having a cancer, the method comprising administering a therapeutically-effective amount of a composition comprising a compound of Formula I:

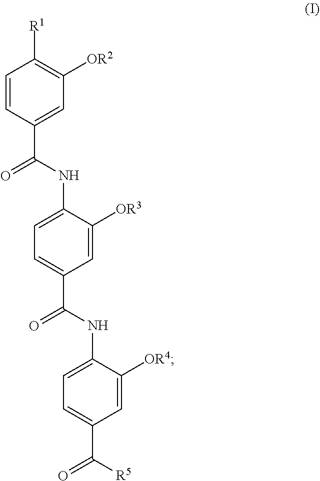

to the subject.

A further aspect of the present disclosure is a composition for use in a method of treating cancer in a subject in need thereof, the method comprising testing a sample from the subject for the presence of elevated Lysosomal lipase A (LIPA) expression and administering a therapeutically-effective amount of a composition comprising a compound of Formula I:

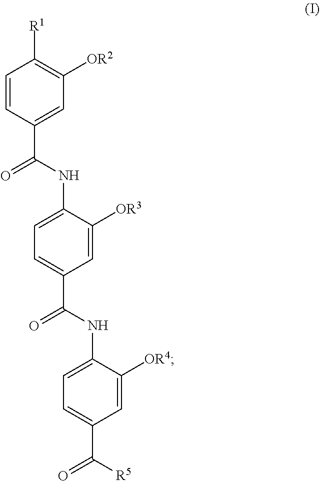

to a subject that provided a sample identified as having an elevated LIPA expression.

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

Additionally, cancer cells that have a high basal level of endoplasmic reticulum stress and that express LIPA are likely to be targeted by these agents. Certain tumor types such as triple negative breast cancer, pancreatic cancer, ovarian and glioblastoma are known to have high basal levels of endoplasmic reticulum stress (detected by higher levels of multiple endoplasmic reticulum stress markers) and have been shown to be responsive to ERX-41 and ERX-315.

In embodiments, the compound of Formula I inhibits LIPA activity when contacted to the LXXLL motif on LIPA.

In various embodiments, the compound is a pharmaceutically acceptable salt of Formula I.

In some embodiments, R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein: R^{1a} is aralkyl_{(C<18)}, substituted aralkyl_{(C<18)}, or the side chain of a canonical amino acid; R², R³, and R⁴ are each independently alkyl_{(C<12)}, substituted alkyl_{(C<12)}, aralkyl_{(C<18)}, or substituted aralkyl_{(C<18)}; and R⁵ is —OR^{5a} or —NHR^{5b}, wherein: R^{5a} is alkyl_{(C<12)} or substituted alkyl_{(C<12)}; R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or a group of the formula:

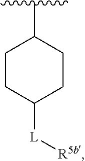

wherein; L is —CO₂— or —C(O)NR^{L}—, wherein: R^{L} hydrogen, alkyl_{(C<12)}, or substituted alkyl_{(C<12)}; R_{5b′} is aryl_{(C<12)}, aralkyl_{(C<18)}, heteroaryl_{(C<12)}, heteroaralkyl_{(C<18)}, or a substituted version of any of these groups.

In several embodiments, the compound comprises Formula I:

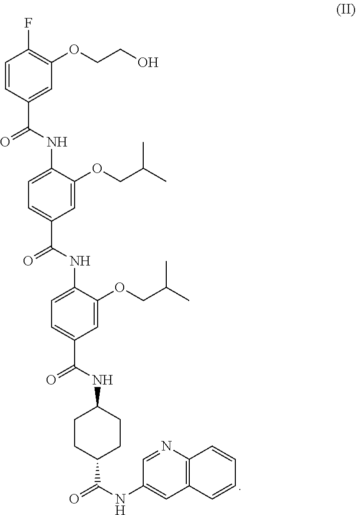 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

In embodiments, the compound comprises Formula III:

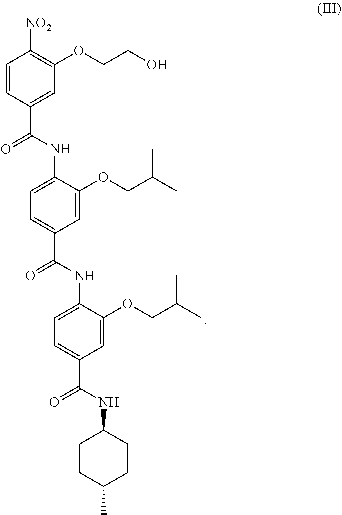 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

In some embodiments, the compound comprises Formula IV:

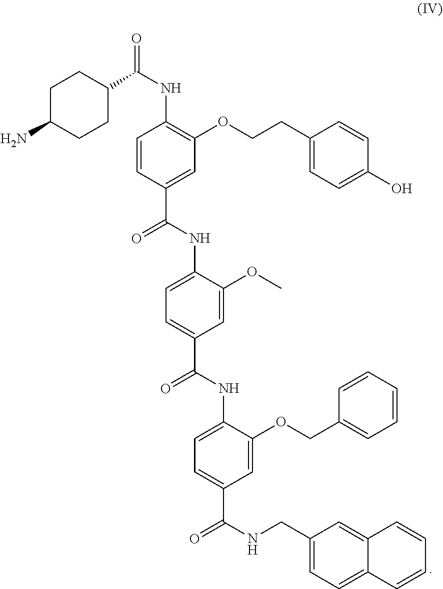

In various embodiments, the method further comprises administering a therapeutically-effective amount of the composition.

In some embodiments, the compound is a pharmaceutically acceptable salt of Formula II, Formula III, or Formula IV.

In several embodiments, the compound of Formula II, Formula III, or Formula IV inhibits LIPA activity when contacted to the LXXLL motif on LIPA.

In embodiments, the compound or composition is administered in an amount sufficient to induce endoplasmic reticulum stress, in an amount sufficient to inhibit activity of LIPA, and/or in an amount sufficient to reduce protein synthesis.

The compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, isotopes of fluorine include ¹⁸F, and isotopes of carbon include ¹³C and ¹⁴C.

In some embodiments, compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

### Methods of Treatment

The compositions and compounds of the present disclosure are useful in treating a cancer in a subject in need thereof. Thus, the present disclosure contemplates in vivo therapeutics for cancer.

An aspect of the present disclosure is a method for treating a cancer characterized by having increased activity of Lysosomal lipase A (LIPA) comprising administering a therapeutically-effective amount of a composition comprising a compound of Formula I:

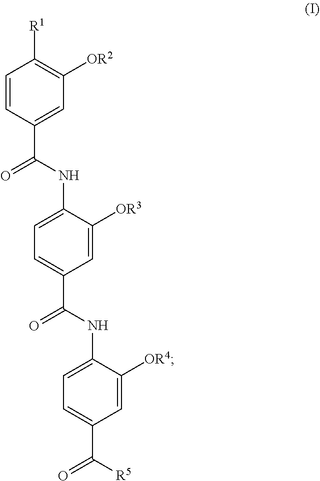

to a subject having a cancer with elevated LIPA expression.

Another aspect of the present disclosure is a method for inhibiting activity of Lysosomal lipase A (LIPA) in a subject having a cancer comprising administering a therapeutically-effective amount of a composition comprising a compound of Formula I:

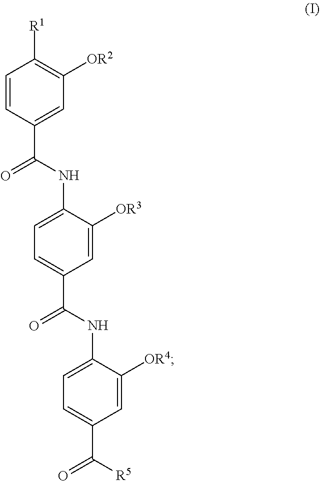

to the subject.

A further aspect of the present disclosure is a method for treating cancer in a subject in need thereof comprising testing a sample from the subject for the presence of elevated Lysosomal lipase A (LIPA) expression and administering a therapeutically-effective amount of a composition comprising a compound of Formula I:

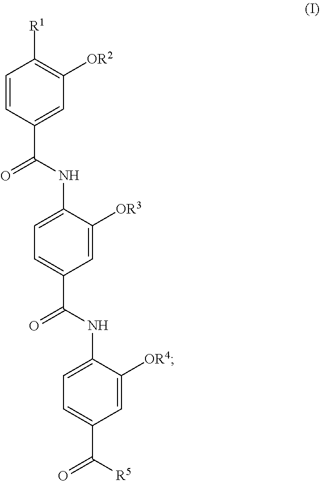

to a subject that provided a sample identified as having an elevated LIPA expression.

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In embodiments, the compound of Formula I inhibits LIPA activity when contacted to the LXXLL motif on LIPA.

In several embodiments, the compound is a pharmaceutically acceptable salt of Formula I.

In various embodiments, R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein: R^{1a} is aralkyl_{(C<18)}, substituted aralkyl_{(C<18)}, or the side chain of a canonical amino acid; R², R³, and R⁴ are each independently alkyl_{(C<12)}, substituted alkyl_{(C<12)}, aralkyl_{(C<18)}, or substituted aralkyl_{(C<18)}; and R⁵ is —OR^{5a} or —NHR^{5b}, wherein: R^{5a} is alkyl_{(C<12)} or substituted alkyl_{(C<12)}; R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or a group of the formula

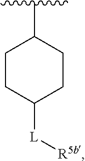

wherein; L is —CO₂— or —C(O)NR^{L}—, wherein: R^{L} hydrogen, alkyl_{(C<12)}, or substituted alkyl_{(C<12)}; R^{5b′} is aryl_{(C<12)}, aralkyl_{(C<18)}, heteroaryl_{(C<12)}, heteroaralkyl_{(C<18)}, or a substituted version of any of these groups.

In embodiments, the composition comprises the compound of Formula II:

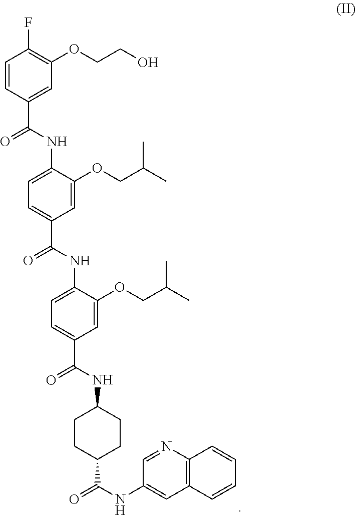 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

In several embodiments, the composition comprises the compound of Formula III:

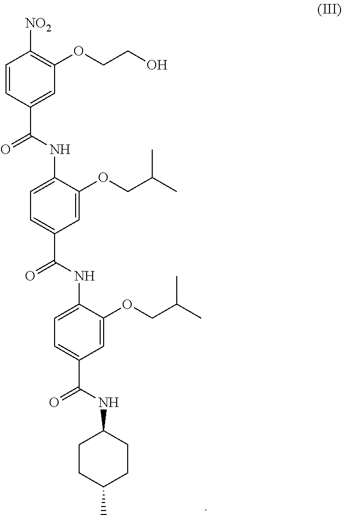 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

In several embodiments, the composition comprises the compound of Formula IV:

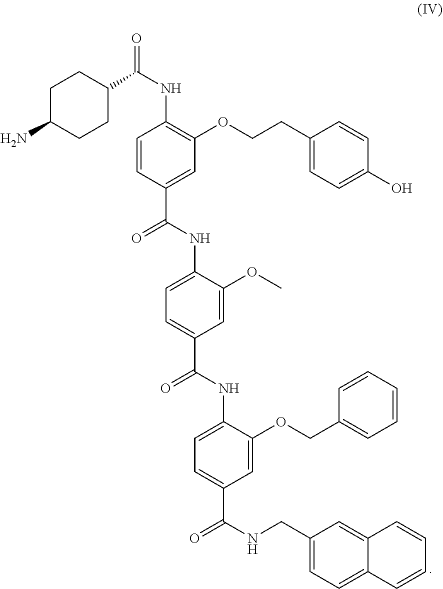

In embodiments, the compound is a pharmaceutically acceptable salt of Formula II, Formula III, or Formula IV.

In various embodiments, the compound of Formula II, Formula III, or Formula IV inhibits LIPA activity when contacted to the LXXLL motif on LIPA.

In some embodiments, the subject is a mammal, (e.g., a human).

In several embodiments, the cancer is a therapy-resistant cancer.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In various embodiments, the administering comprises in vivo administration, (e.g., intravenous, intra-arterial, intra-tumoral, subcutaneous, topical, or intraperitoneal administration).

In some embodiments, the administering comprises local, regional, systemic, or continual administration.

In several embodiments, the method further comprises providing to a subject a second anti-cancer therapy. In some cases, the second anti-cancer therapy is surgery, chemotherapy, radiotherapy, hormonal therapy, toxin therapy, immunotherapy, and cryotherapy. The second anti-cancer therapy may be provided prior to administering the composition, the second anti-cancer therapy may be provided after administering the composition, and/or the second anti-cancer therapy may be provided contemporaneous with the composition.

In embodiments, the composition is administered daily, e.g., daily for 7 days, 2 weeks, 3 weeks, 4 weeks, one month, 6 weeks, 8 weeks, two months, 12 weeks, or 3 months.

In various embodiments, the composition is administered weekly, e.g., weekly for 2 weeks, 3 weeks, 4 weeks, 6 weeks, 8 weeks, 10 weeks, or 12 weeks.

In some embodiments, the composition is administered intermitantly.

In some embodiments, the compound or composition is administered in an amount sufficient to induce endoplasmic reticulum stress, in an amount sufficient to inhibit activity of LIPA, and/or in an amount sufficient to reduce protein synthesis.

The present disclosure also provides a composition for use in any herein-disclosed method.

In an aspect, the present disclosure provides a method for treating a cancer characterized by having increased activity of Lysosomal lipase A (LIPA) comprising administering a therapeutically-effective amount of a composition comprising the compound of Formula II:

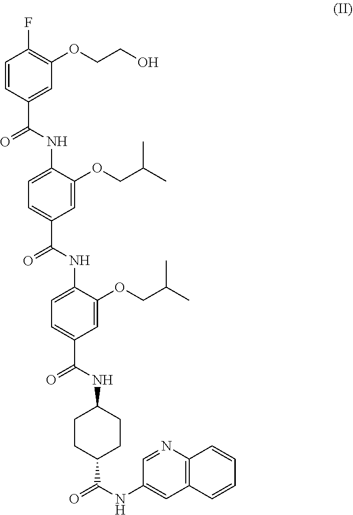 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

to a subject having a cancer with elevated LIPA expression.

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In several embodiments, the compound is a pharmaceutically acceptable salt of Formula II.

In embodiments, the compound of Formula II inhibits LIPA activity when contacted to the LXXLL motif on LIPA.

In embodiments, the subject is a mammal (e.g., a human).

In embodiments, the cancer is a therapy-resistant cancer.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In embodiments, the administering comprises in vivo administration (e.g., intravenous, intra-arterial, intra-tumoral, subcutaneous, topical, or intraperitoneal administration).

In embodiments, the administering comprises local, regional, systemic, or continual administration.

In embodiments, the method further comprises providing to a subject a second anti-cancer therapy. In some cases, the second anti-cancer therapy is surgery, chemotherapy, radiotherapy, hormonal therapy, toxin therapy, immunotherapy, and cryotherapy. The second anti-cancer therapy may be provided prior to administering the composition, the second anti-cancer therapy may be provided after administering the composition, and/or the second anti-cancer therapy may be provided contemporaneous with the composition.

In embodiments, the composition is administered daily (e.g., daily for 7 days, 2 weeks, 3 weeks, 4 weeks, one month, 6 weeks, 8 weeks, two months, 12 weeks, or 3 months).

In embodiments, the composition is administered weekly (e.g., weekly for 2 weeks, 3 weeks, 4 weeks, 6 weeks, 8 weeks, 10 weeks, or 12 weeks).

In embodiments, the compound or composition is administered in an amount sufficient to induce endoplasmic reticulum stress, in an amount sufficient to inhibit activity of LIPA, and/or in an amount sufficient to reduce protein synthesis.

In another aspect, the present disclosure provides a method for inhibiting activity of Lysosomal lipase A (LIPA) in a subject having a cancer comprising administering a therapeutically-effective amount of a composition comprising the compound of Formula II:

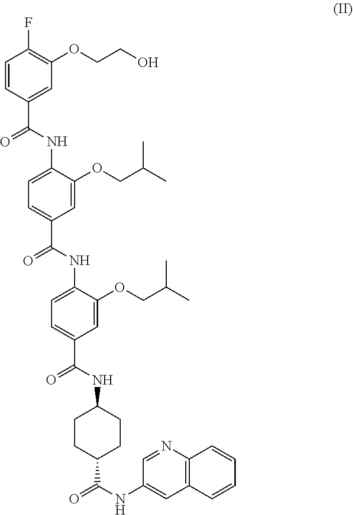 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

to the subject.

In some embodiments, the compound is a pharmaceutically acceptable salt of Formula II.

In several embodiments, the compound of Formula II inhibits LIPA activity when contacted to the LXXLL motif on LIPA.

In embodiments, the subject is a mammal (e.g., a human).

In embodiments, the cancer is a therapy-resistant cancer.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In embodiments, the administering comprises in vivo administration (e.g., intravenous, intra-arterial, intra-tumoral, subcutaneous, topical, or intraperitoneal administration).

In embodiments, the administering comprises local, regional, systemic, or continual administration.

In embodiments, the method further comprises providing to a subject a second anti-cancer therapy. In some cases, the second anti-cancer therapy is surgery, chemotherapy, radiotherapy, hormonal therapy, toxin therapy, immunotherapy, and cryotherapy. The second anti-cancer therapy may be provided prior to administering the composition, the second anti-cancer therapy may be provided after administering the composition, and/or the second anti-cancer therapy may be provided contemporaneous with the composition.

In embodiments, the composition is administered daily (e.g., daily for 7 days, 2 weeks, 3 weeks, 4 weeks, one month, 6 weeks, 8 weeks, two months, 12 weeks, or 3 months).

In embodiments, the composition is administered weekly (e.g., weekly for 2 weeks, 3 weeks, 4 weeks, 6 weeks, 8 weeks, 10 weeks, or 12 weeks).

In embodiments, the compound or composition is administered in an amount sufficient to induce endoplasmic reticulum stress, in an amount sufficient to inhibit activity of LIPA, and/or in an amount sufficient to reduce protein synthesis.

In a further aspect, the present disclosure provides a method for treating cancer in a subject in need thereof comprising testing a sample from the subject for the presence of elevated Lysosomal lipase A (LIPA) expression and administering a therapeutically-effective amount of a composition comprising the compound of Formula II:

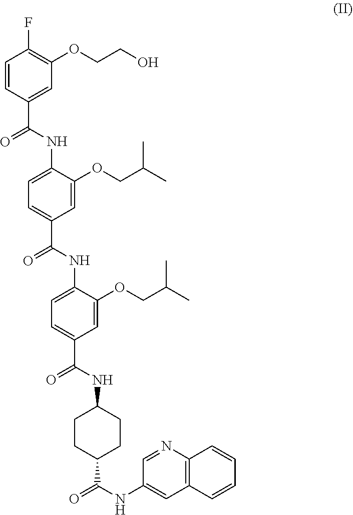 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

to a subject that provided a sample identified as having an elevated LIPA expression.

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In various embodiments, the compound is a pharmaceutically acceptable salt of Formula II.

In some embodiments, the compound of Formula II inhibits LIPA activity when contacted to the LXXLL motif on LIPA.

In embodiments, the subject is a mammal, e.g., a human.

In embodiments, the cancer is a therapy-resistant cancer.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In embodiments, the administering comprises in vivo administration (e.g., intravenous, intra-arterial, intra-tumoral, subcutaneous, topical, or intraperitoneal administration).

In embodiments, the administering comprises local, regional, systemic, or continual administration.

In embodiments, the method further comprises providing to a subject a second anti-cancer therapy. In some cases, the second anti-cancer therapy is surgery, chemotherapy, radiotherapy, hormonal therapy, toxin therapy, immunotherapy, and cryotherapy. The second anti-cancer therapy may be provided prior to administering the composition, the second anti-cancer therapy may be provided after administering the composition, and/or the second anti-cancer therapy may be provided contemporaneous with the composition.

In embodiments, the composition is administered daily (e.g., daily for 7 days, 2 weeks, 3 weeks, 4 weeks, one month, 6 weeks, 8 weeks, two months, 12 weeks, or 3 months).

In embodiments, the composition is administered weekly (e.g., weekly for 2 weeks, 3 weeks, 4 weeks, 6 weeks, 8 weeks, 10 weeks, or 12 weeks).

In embodiments, the compound or composition is administered in an amount sufficient to induce endoplasmic reticulum stress, in an amount sufficient to inhibit activity of LIPA, and/or in an amount sufficient to reduce protein synthesis.

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, (e.g., for oral administration) the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

In some embodiments, a composition of the present disclosure is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in crimes, in lipid compositions, via a catheter, via a lavage, via continuous infusion, via infusion, via inhalation, via injection, via local delivery, or via localized perfusion. In further embodiments, the composition is formulated for administration: orally, intraarterially, intratumorally, intravenously, locally, subcutaneously, topically, intraperitoneally, or via injection.

Compositions of the present disclosure may be administered by a variety of methods including orally or by injection (e.g., subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the patient has eaten or will eat.

### Breast Cancer

Breast cancer refers to cancers originating from breast tissue, most commonly from the inner lining of milk ducts or the lobules that supply the ducts with milk. Cancers originating from ducts are known as ductal carcinomas; those originating from lobules are known as lobular carcinomas. There are many different types of breast cancer, with different stages (spread), aggressiveness, and genetic makeup; survival varies greatly depending on those factors. Computerized models are available to predict survival. With best treatment and dependent on staging, 10-year disease-free survival varies from 98% to 10%. Treatment includes surgery, drugs (hormonal therapy and chemotherapy), and radiation.

Worldwide, breast cancer comprises 10.4% of all cancer incidence among women, making it the second most common type of non-skin cancer (after lung cancer) and the fifth most common cause of cancer death. In 2004, breast cancer caused 519,000 deaths worldwide (7% of cancer deaths; almost 1% of all deaths). Breast cancer is about 100 times more common in women than in men, although males tend to have poorer outcomes due to delays in diagnosis.

Some breast cancers require the hormones estrogen and progesterone to grow, and have receptors for those hormones. After surgery those cancers are treated with drugs that interfere with those hormones, usually tamoxifen, and with drugs that shut off the production of estrogen in the ovaries or elsewhere; this may damage the ovaries and end fertility. After surgery, low-risk, hormone-sensitive breast cancers may be treated with hormone therapy and radiation alone. Breast cancers without hormone receptors, or which have spread to the lymph nodes in the armpits, or which express certain genetic characteristics, are higher-risk, and are treated more aggressively. One standard regimen, popular in the U.S., is cyclophosphamide plus doxorubicin (Adriamycin), known as CA; these drugs damage DNA in the cancer, but also in fast-growing normal cells where they cause serious side effects. Sometimes a taxane drug, such as docetaxel, is added, and the regime is then known as CAT; taxane attacks the microtubules in cancer cells. An equivalent treatment, popular in Europe, is cyclophosphamide, methotrexate, and fluorouracil (CMF). Monoclonal antibodies, such as trastuzumab (Herceptin), are used for cancer cells that have the HER2 mutation. Radiation is usually added to the surgical bed to control cancer cells that were missed by the surgery, which usually extends survival, although radiation exposure to the heart may cause damage and heart failure in the following years.

While screening techniques (which are further discussed below) are useful in determining the possibility of cancer, a further testing is necessary to confirm whether a lump detected on screening is cancer, as opposed to a benign alternative such as a simple cyst.

In a clinical setting, breast cancer is commonly diagnosed using a “triple test” of clinical breast examination (breast examination by a trained medical practitioner), mammography, and fine needle aspiration cytology. Both mammography and clinical breast exam, also used for screening, can indicate an approximate likelihood that a lump is cancer, and may also identify any other lesions. Fine Needle Aspiration and Cytology (FNAC), which may be done in a doctor's office using local anesthetic if required, involves attempting to extract a small portion of fluid from the lump. Clear fluid makes the lump highly unlikely to be cancerous, but bloody fluid may be sent off for inspection under a microscope for cancerous cells. Together, these three tools can be used to diagnose breast cancer with a good degree of accuracy.

Other options for biopsy include core biopsy, where a section of the breast lump is removed, and an excisional biopsy, where the entire lump is removed.

In addition vacuum-assisted breast biopsy (VAB) may help diagnose breast cancer among patients with a mammographically detected breast in women according to a systematic review. In this study, summary estimates for vacuum assisted breast biopsy in diagnosis of breast cancer were as follows sensitivity was 98.1% with 95% CI=0.972-0.987 and specificity was 100% with 95% CI=0.997-0.999; however, underestimate rates of atypical ductal hyperplasia (ADH) and ductal carcinoma in situ (DCIS) were 20.9% with 95% CI=0.177-0.245 and 11.2% with 95% CI=0.098-0.128 respectively.

Breast cancer screening refers to testing otherwise-healthy women for breast cancer in an attempt to achieve an earlier diagnosis. The assumption is that early detection will improve outcomes. A number of screening tests have been employed including: clinical and self breast exams, mammography, genetic screening, ultrasound, and magnetic resonance imaging.

A clinical or self breast exam involves feeling the breast for lumps or other abnormalities. Research evidence does not support the effectiveness of either type of breast exam, because by the time a lump is large enough to be found it is likely to have been growing for several years and will soon be large enough to be found without an exam. Mammographic screening for breast cancer uses x-rays to examine the breast for any uncharacteristic masses or lumps. In women at high risk, such as those with a strong family history of cancer, mammography screening is recommended at an earlier age and additional testing may include genetic screening that tests for the BRCA genes and/or magnetic resonance imaging.

Breast cancer is sometimes treated first with surgery, and then with chemotherapy, radiation, or both. Treatments are given with increasing aggressiveness according to the prognosis and risk of recurrence. Stage 1 cancers (and DCIS) have an excellent prognosis and are generally treated with lumpectomy with or without chemotherapy or radiation. Although the aggressive HER2+ cancers should also be treated with the trastuzumab (Herceptin) regime. Stage 2 and 3 cancers with a progressively poorer prognosis and greater risk of recurrence are generally treated with surgery (lumpectomy or mastectomy with or without lymph node removal), radiation (sometimes) and chemotherapy (plus trastuzumab for HER2+ cancers). Stage 4, metastatic cancer, (i.e., spread to distant sites) is not curable and is managed by various combinations of all treatments from surgery, radiation, chemotherapy and targeted therapies. These treatments increase the median survival time of stage 4 breast cancer by about 6 months.

Breast cancer subtypes are typically categorized on an immunohistochemical basis. Subtype definitions are generally as follows:
normal (ER+, PR+, HER2+, cytokeratin 5/6+, and HER1+)
luminal A (ER+ and/or PR+, HER2−)
luminal B (ER+ and/or PR+, HER2+)
triple-negative (ER−, PR−, HER2−)
HER2+/ER− (ER−, PR−, and HER2+)
unclassified (ER−, PR−, HER2−, cytokeratin 5/6−, and HER1−)
   In the case of triple-negative breast cancer cells, the cancer's growth is not driven by estrogen or progesterone, or by growth signals coming from the HER2 protein. By the same token, such cancer cells do not respond to hormonal therapy, such as tamoxifen or aromatase inhibitors, or therapies that target HER2 receptors, such as Herceptin®. About 10-20% of breast cancers are found to be triple-negative. It is important to identify these types of cancer to that one can avoid costly and toxic effects of therapies that are unlike to succeed, and to focus on treatments that can be used to treat triple-negative breast cancer. Like other forms of breast cancer, triple-negative breast cancer can be treated with surgery, radiation therapy, and/or chemotherapy. One particularly promising approach is “neoadjuvant” therapy, where chemo- and/or radiotherapy is provided prior to surgery. Another drug therapy is the use of poly (ADP-ribose) polymerase, or PARP inhibitors.

### Ovarian Cancer

Ovarian cancer is a cancerous growth arising from different parts of the ovary. Most (>90%) ovarian cancers are classified as “epithelial” and were believed to arise from the surface (epithelium) of the ovary. However, recent evidence suggests that the Fallopian tube could also be the source of some ovarian cancers. Since the ovaries and tubes are closely related to each other, it is hypothesized that these cells can mimic ovarian cancer. Other types arise from the egg cells (germ cell tumor) or supporting cells (sex cord/stromal).

In 2004, in the United States, 25,580 new cases were diagnosed and 16,090 women died of ovarian cancer. The risk increases with age and decreases with pregnancy. Lifetime risk is about 1.6%, but women with affected first-degree relatives have a 5% risk. Women with a mutated BRCA1 or BRCA2 gene carry a risk between 25% and 60% depending on the specific mutation. Ovarian cancer is the fifth leading cause of death from cancer in women and the leading cause of death from gynecological cancer.

Ovarian cancer causes non-specific symptoms. Early diagnosis would result in better survival, on the assumption that stage I and II cancers progress to stage III and IV cancers (but this has not been proven). Most women with ovarian cancer report one or more symptoms such as abdominal pain or discomfort, an abdominal mass, bloating, back pain, urinary urgency, constipation, tiredness and a range of other non-specific symptoms, as well as more specific symptoms such as pelvic pain, abnormal vaginal bleeding or involuntary weight loss. There can be a build-up of fluid (ascites) in the abdominal cavity.

Diagnosis of ovarian cancer starts with a physical examination (including a pelvic examination), a blood test (for CA-125 and sometimes other markers), and transvaginal ultrasound. The diagnosis must be confirmed with surgery to inspect the abdominal cavity, take biopsies (tissue samples for microscopic analysis) and look for cancer cells in the abdominal fluid. Treatment usually involves chemotherapy and surgery, and sometimes radiotherapy.

In most cases, the cause of ovarian cancer remains unknown. Older women, and in those who have a first or second degree relative with the disease, have an increased risk. Hereditary forms of ovarian cancer can be caused by mutations in specific genes (most notably BRCA1 and BRCA2, but also in genes for hereditary nonpolyposis colorectal cancer). Infertile women and those with a condition called endometriosis, those who have never been pregnant and those who use postmenopausal estrogen replacement therapy are at increased risk. Use of combined oral contraceptive pills is a protective factor. The risk is also lower in women who have had their uterine tubes blocked surgically (tubal ligation).

Ovarian cancer is classified according to the histology of the tumor, obtained in a pathology report. Histology dictates many aspects of clinical treatment, management, and prognosis. Surface epithelial-stromal tumor, also known as ovarian epithelial carcinoma, is the most common type of ovarian cancer. It includes serous tumor, endometrioid tumor and mucinous cystadenocarcinoma. Sex cord-stromal tumor, including estrogen-producing granulosa cell tumor and virilizing Sertoli-Leydig cell tumor or arrhenoblastoma, accounts for 8% of ovarian cancers. Germ cell tumor accounts for approximately 30% of ovarian tumors but only 5% of ovarian cancers, because most germ cell tumors are teratomas and most teratomas are benign (see Teratoma). Germ cell tumor tends to occur in young women and girls. The prognosis depends on the specific histology of germ cell tumor, but overall is favorable. Mixed tumors, containing elements of more than one of the above classes of tumor histology.

Ovarian cancer can also be a secondary cancer, the result of metastasis from a primary cancer elsewhere in the body. Seven percent of ovarian cancers are due to metastases while the rest are primary cancers. Common primary cancers are breast cancer and gastrointestinal cancer (a common mistake is to name all peritoneal metastases from any gastrointestinal cancer as Krukenberg cancer, but this is only the case if it originates from primary gastric cancer). Surface epithelial-stromal tumor can originate in the peritoneum (the lining of the abdominal cavity), in which case the ovarian cancer is secondary to primary peritoneal cancer, but treatment is basically the same as for primary surface epithelial-stromal tumor involving the peritoneum.

Ovarian cancer staging is by the FIGO staging system and uses information obtained after surgery, which can include a total abdominal hysterectomy, removal of (usually) both ovaries and fallopian tubes, (usually) the omentum, and pelvic (peritoneal) washings for cytopathology. The AJCC stage is the same as the FIGO stage. The AJCC staging system describes the extent of the primary Tumor (T), the absence or presence of metastasis to nearby lymph Nodes (N), and the absence or presence of distant Metastasis (M).

The AJCC/TNM staging system includes three categories for ovarian cancer, T, N and M. The T category contains three other subcategories, T1, T2 and T3, each of them being classified according to the place where the tumor has developed (in one or both ovaries, inside or outside the ovary). The T1 category of ovarian cancer describes ovarian tumors that are confined to the ovaries, and which may affect one or both of them. The sub-subcategory T1a is used to stage cancer that is found in only one ovary, which has left the capsule intact and which cannot be found in the fluid taken from the pelvis. Cancer that has not affected the capsule, is confined to the inside of the ovaries and cannot be found in the fluid taken from the pelvis but has affected both ovaries is staged as T1b. T1c category describes a type of tumor that can affect one or both ovaries, and which has grown through the capsule of an ovary or it is present in the fluid taken from the pelvis. T2 is a more advanced stage of cancer. In this case, the tumor has grown in one or both ovaries and is spread to the uterus, fallopian tubes or other pelvic tissues. Stage T2a is used to describe a cancerous tumor that has spread to the uterus or the fallopian tubes (or both) but which is not present in the fluid taken from the pelvis. Stages T2b and T2c indicate cancer that metastasized to other pelvic tissues than the uterus and fallopian tubes and which cannot be seen in the fluid taken from the pelvis, respectively tumors that spread to any of the pelvic tissues (including uterus and fallopian tubes) but which can also be found in the fluid taken from the pelvis. T3 is the stage used to describe cancer that has spread to the peritoneum. This stage provides information on the size of the metastatic tumors (tumors that are located in other areas of the body, but are caused by ovarian cancer). These tumors can be very small, visible only under the microscope (T3a), visible but not larger than 2 centimeters (T3b) and bigger than 2 centimeters (T3c).

This staging system also uses N categories to describe cancers that have or not spread to nearby lymph nodes. There are only two N categories, N0 which indicates that the cancerous tumors have not affected the lymph nodes, and N1 which indicates the involvement of lymph nodes close to the tumor. The M categories in the AJCC/TNM staging system provide information on whether the ovarian cancer has metastasized to distant organs such as liver or lungs. M0 indicates that the cancer did not spread to distant organs and M1 category is used for cancer that has spread to other organs of the body. The AJCC/TNM staging system also contains a Tx and a Nx sub-category which indicates that the extent of the tumor cannot be described because of insufficient data, respectively the involvement of the lymph nodes cannot be described because of the same reason.

Ovarian cancer, as well as any other type of cancer, is also graded, apart from staged. The histologic grade of a tumor measures how abnormal or malignant its cells look under the microscope. There are four grades indicating the likelihood of the cancer to spread and the higher the grade, the more likely for this to occur. Grade 0 is used to describe non-invasive tumors. Grade 0 cancers are also referred to as borderline tumors. Grade 1 tumors have cells that are well differentiated (look very similar to the normal tissue) and are the ones with the best prognosis. Grade 2 tumors are also called moderately well differentiated and they are made up by cells that resemble the normal tissue. Grade 3 tumors have the worst prognosis and their cells are abnormal, referred to as poorly differentiated.

The signs and symptoms of ovarian cancer are most of the times absent, but when they exist they are nonspecific. In most cases, the symptoms persist for several months until the patient is diagnosed.

A prospective case-control study of 1,709 women visiting primary care clinics found that the combination of bloating, increased abdominal size, and urinary symptoms was found in 43% of those with ovarian cancer but in only 8% of those presenting to primary care clinics.

The exact cause is usually unknown. The risk of developing ovarian cancer appears to be affected by several factors. The more children a woman has, the lower her risk of ovarian cancer. Early age at first pregnancy, older age of final pregnancy and the use of low dose hormonal contraception have also been shown to have a protective effect. Ovarian cancer is reduced in women after tubal ligation.

The relationship between use of oral contraceptives and ovarian cancer was shown in a summary of results of 45 case-control and prospective studies. Cumulatively these studies show a protective effect for ovarian cancers. Women who used oral contraceptives for 10 years had about a 60% reduction in risk of ovarian cancer. (risk ratio 0.42 with statistical significant confidence intervals given the large study size, not unexpected). This means that if 250 women took oral contraceptives for 10 years, 1 ovarian cancer would be prevented. This is by far the largest epidemiological study to date on this subject (45 studies, over 20,000 women with ovarian cancer and about 80,000 controls).

The link to the use of fertility medication, such as Clomiphene citrate, has been controversial. An analysis in 1991 raised the possibility that use of drugs may increase the risk of ovarian cancer. Several cohort studies and case-control studies have been conducted since then without demonstrating conclusive evidence for such a link. It will remain a complex topic to study as the infertile population differs in parity from the “normal” population.

There is good evidence that in some women genetic factors are important. Carriers of certain mutations of the BRCA1 or the BRCA2 gene are notably at risk. The BRCA1 and BRCA2 genes account for 5%-13% of ovarian cancers and certain populations (e.g., Ashkenazi Jewish women) are at a higher risk of both breast cancer and ovarian cancer, often at an earlier age than the general population. Patients with a personal history of breast cancer or a family history of breast and/or ovarian cancer, especially if diagnosed at a young age, may have an elevated risk.

A strong family history of uterine cancer, colon cancer, or other gastrointestinal cancers may indicate the presence of a syndrome known as hereditary nonpolyposis colorectal cancer (HNPCC, also known as Lynch syndrome), which confers a higher risk for developing ovarian cancer. Patients with strong genetic risk for ovarian cancer may consider the use of prophylactic, i.e., preventative, oophorectomy after completion of childbearing. Australia being member of International Cancer Genome Consortium is leading efforts to map ovarian cancer's complete genome.

Ovarian cancer at its early stages (I/II) is difficult to diagnose until it spreads and advances to later stages (III/IV). This is because most symptoms are non-specific and thus of little use in diagnosis.

When an ovarian malignancy is included in the list of diagnostic possibilities, a limited number of laboratory tests are indicated. A complete blood count (CBC) and serum electrolyte test should be obtained in all patients.

The serum BHCG level should be measured in any female in whom pregnancy is a possibility. In addition, serum alpha-fetoprotein (AFP) and lactate dehydrogenase (LDH) should be measured in young girls and adolescents with suspected ovarian tumors because the younger the patient, the greater the likelihood of a malignant germ cell tumor.

A blood test called CA-125 is useful in differential diagnosis and in follow up of the disease, but it by itself has not been shown to be an effective method to screen for early-stage ovarian cancer due to its unacceptable low sensitivity and specificity. However, this is the only widely-used marker currently available.

Current research is looking at ways to combine tumor markers proteomics along with other indicators of disease (i.e., radiology and/or symptoms) to improve accuracy. The challenge in such an approach is that the very low population prevalence of ovarian cancer means that even testing with very high sensitivity and specificity will still lead to a number of false positive results (i.e., performing surgical procedures in which cancer is not found intra-operatively). However, the contributions of proteomics are still in the early stages and require further refining. Current studies on proteomics mark the beginning of a paradigm shift towards individually tailored therapy.

A pelvic examination and imaging including CT scan and trans-vaginal ultrasound are essential. Physical examination may reveal increased abdominal girth and/or ascites (fluid within the abdominal cavity). Pelvic examination may reveal an ovarian or abdominal mass. The pelvic examination can include a rectovaginal component for better palpation of the ovaries. For very young patients, magnetic resonance imaging may be preferred to rectal and vaginal examination.

To definitively diagnose ovarian cancer, a surgical procedure to take a look into the abdomen is required. This can be an open procedure (laparotomy, incision through the abdominal wall) or keyhole surgery (laparoscopy). During this procedure, suspicious areas will be removed and sent for microscopic analysis. Fluid from the abdominal cavity can also be analyzed for cancerous cells. If there is cancer, this procedure can also determine its spread (which is a form of tumor staging).

Women who have had children are less likely to develop ovarian cancer than women who have not, and breastfeeding may also reduce the risk of certain types of ovarian cancer. Tubal ligation and hysterectomy reduce the risk and removal of both tubes and ovaries (bilateral salpingo-oophorectomy) dramatically reduces the risk of not only ovarian cancer but breast cancer also. The use of oral contraceptives (birth control pills) for five years or more decreases the risk of ovarian cancer in later life by 50%.

Tubal ligation is believed to decrease the chance of developing ovarian cancer by up to 67% while a hysterectomy may reduce the risk of getting ovarian cancer by about one-third. Moreover, according to some studies, analgesics such as acetaminophen and aspirin seem to reduce one's risks of developing ovarian cancer. Yet, the information is not consistent and more research needs to be carried on this matter.

Routine screening of women for ovarian cancer is not recommended by any professional society—this includes the U.S. Preventive Services Task Force, the American Cancer Society, the American College of Obstetricians and Gynecologists, and the National Comprehensive Cancer Network. This is because no trial has shown improved survival for women undergoing screening. Screening for any type of cancer must be accurate and reliable—it needs to accurately detect the disease and it must not give false positive results in people who do not have cancer. As yet there is no technique for ovarian screening that has been shown to fulfil these criteria. However, in some countries such as the UK, women who are likely to have an increased risk of ovarian cancer (for example if they have a family history of the disease) can be offered individual screening through their doctors, although this will not necessarily detect the disease at an early stage.

Researchers are assessing different ways to screen for ovarian cancer. Screening tests that could potentially be used alone or in combination for routine screening include the CA-125 marker and transvaginal ultrasound. Doctors can measure the levels of the CA-125 protein in a woman's blood—high levels could be a sign of ovarian cancer, but this is not always the case. And not all women with ovarian cancer have high CA-125 levels. Transvaginal ultrasound involves using an ultrasound probe to scan the ovaries from inside the vagina, giving a clearer image than scanning the abdomen. The UK Collaborative Trial of Ovarian Cancer Screening is testing a screening technique that combines CA-125 blood tests with transvaginal ultrasound.

The purpose of screening is to diagnose ovarian cancer at an early stage, when it is more likely to be treated successfully. However, the development of the disease is not fully understood, and it has been argued that early-stage cancers may not always develop into late-stage disease. With any screening technique there are risks and benefits that need to be carefully considered, and health authorities need to assess these before introducing any ovarian cancer screening programs.

The goal of ovarian cancer screening is to detect the disease at stage I. Several large studies are ongoing, but none have identified an effective technique. In 2009, however, early results from the UK Collaborative Trial of Ovarian Cancer Screening (UKCTOCS) showed that a technique combining annual CA-125 tests with ultrasound imaging did help to detect the disease at an early stage. However, it is not yet clear if this approach could actually help to save lives—the full results of the trial will be published in 2015.

Surgical treatment may be sufficient for malignant tumors that are well-differentiated and confined to the ovary. Addition of chemotherapy may be required for more aggressive tumors that are confined to the ovary. For patients with advanced disease a combination of surgical reduction with a combination chemotherapy regimen is standard. Borderline tumors, even following spread outside of the ovary, are managed well with surgery, and chemotherapy is not seen as useful.

Surgery is the preferred treatment and is frequently necessary to obtain a tissue specimen for differential diagnosis via its histology. Surgery performed by a specialist in gynecologic oncology usually results in an improved result. Improved survival is attributed to more accurate staging of the disease and a higher rate of aggressive surgical excision of tumor in the abdomen by gynecologic oncologists as opposed to general gynecologists and general surgeons.

The type of surgery depends upon how widespread the cancer is when diagnosed (the cancer stage), as well as the presumed type and grade of cancer. The surgeon may remove one (unilateral oophorectomy) or both ovaries (bilateral oophorectomy), the fallopian tubes (salpingectomy), and the uterus (hysterectomy). For some very early tumors (stage 1, low grade or low-risk disease), only the involved ovary and fallopian tube will be removed (called a “unilateral salpingo-oophorectomy,” USO), especially in young females who wish to preserve their fertility.

In advanced malignancy, where complete resection is not feasible, as much tumor as possible is removed (debulking surgery). In cases where this type of surgery is successful (i.e., <1 cm in diameter of tumor is left behind [“optimal debulking” ]), the prognosis is improved compared to patients where large tumor masses (>1 cm in diameter) are left behind. Minimally invasive surgical techniques may facilitate the safe removal of very large (greater than 10 cm) tumors with fewer complications of surgery.

Chemotherapy has been a general standard of care for ovarian cancer for decades, although with highly variable protocols. Chemotherapy is used after surgery to treat any residual disease, if appropriate. This depends on the histology of the tumor; some kinds of tumor (particularly teratoma) are not sensitive to chemotherapy. In some cases, there may be reason to perform chemotherapy first, followed by surgery.

For patients with stage IIIC epithelial ovarian adenocarcinomas who have undergone successful optimal debulking, a recent clinical trial demonstrated that median survival time is significantly longer for patient receiving intraperitoneal (IP) chemotherapy. Patients in this clinical trial reported less compliance with IP chemotherapy and fewer than half of the patients received all six cycles of IP chemotherapy. Despite this high “drop-out” rate, the group as a whole (including the patients that didn't complete IP chemotherapy treatment) survived longer on average than patients who received intravenous chemotherapy alone.

Some specialists believe the toxicities and other complications of IP chemotherapy will be unnecessary with improved IV chemotherapy drugs currently being developed.

Although IP chemotherapy has been recommended as a standard of care for the first-line treatment of ovarian cancer, the basis for this recommendation has been challenged.

Radiation therapy is not effective for advanced stages because when vital organs are in the radiation field, a high dose cannot be safely delivered. Radiation therapy is then commonly avoided in such stages as the vital organs may not be able to withstand the problems associated with these ovarian cancer treatments.

Ovarian cancer usually has a poor prognosis. It is disproportionately deadly because it lacks any clear early detection or screening test, meaning that most cases are not diagnosed until they have reached advanced stages. More than 60% of women presenting with this cancer already have stage III or stage IV cancer, when it has already spread beyond the ovaries. Ovarian cancers shed cells into the naturally occurring fluid within the abdominal cavity. These cells can then implant on other abdominal (peritoneal) structures, included the uterus, urinary bladder, bowel and the lining of the bowel wall omentum forming new tumor growths before cancer is even suspected.

The five-year survival rate for all stages of ovarian cancer is 45.5%. For cases where a diagnosis is made early in the disease, when the cancer is still confined to the primary site, the five-year survival rate is 92.7%.

### Brain Cancer

A brain tumor is an intracranial solid neoplasm, a tumor (defined as an abnormal growth of cells) within the brain or the central spinal canal. Brain tumors include all tumors inside the cranium or in the central spinal canal. They are created by an abnormal and uncontrolled cell division, normally either in the brain itself (neurons, glial cells (astrocytes, oligodendrocytes, ependymal cells, myelin-producing Schwann cells), lymphatic tissue, blood vessels), in the cranial nerves, in the brain envelopes (meninges), skull, pituitary and pineal gland, or spread from cancers primarily located in other organs (metastatic tumors).

Any brain tumor is inherently serious and life-threatening because of its invasive and infiltrative character in the limited space of the intracranial cavity. However, brain tumors (even malignant ones) are not invariably fatal. Brain tumors or intracranial neoplasms can be cancerous (malignant) or non-cancerous (benign); however, the definitions of malignant or benign neoplasms differs from those commonly used in other types of cancerous or non-cancerous neoplasms in the body. Its threat level depends on the combination of factors like the type of tumor, its location, its size and its state of development. Because the brain is well protected by the skull, the early detection of a brain tumor only occurs when diagnostic tools are directed at the intracranial cavity. Usually detection occurs in advanced stages when the presence of the tumor has caused unexplained symptoms.

Primary (true) brain tumors are commonly located in the posterior cranial fossa in children and in the anterior two-thirds of the cerebral hemispheres in adults, although they can affect any part of the brain.

The prognosis of brain cancer varies based on the type of cancer. Medulloblastoma has a good prognosis with chemotherapy, radiotherapy, and surgical resection while glioblastoma multiforme has a median survival of only 12 months even with aggressive chemoradiotherapy and surgery. Brainstem gliomas have the poorest prognosis of any form of brain cancer, with most patients dying within one year, even with therapy that typically consists of radiation to the tumor along with corticosteroids. However, one type of brainstem glioma, a focal seems open to exceptional prognosis and long-term survival has frequently been reported.

Glioblastoma multiforme is the deadliest and most common form of malignant brain tumor. Even when aggressive multimodality therapy consisting of radiotherapy, chemotherapy, and surgical excision is used, median survival is only 12-17 months. Standard therapy for glioblastoma multiforme consists of maximal surgical resection of the tumor, followed by radiotherapy between two and four weeks after the surgical procedure to remove the cancer. This is followed by chemotherapy. Most patients with glioblastoma take a corticosteroid, typically dexamethasone, during their illness to palliate symptoms. Experimental treatments include gamma-knife radiosurgery, boron neutron capture therapy and gene transfer.

Oligodendroglioma is an incurable but slowly progressive malignant brain tumor. They can be treated with surgical resection, chemotherapy, and/or radiotherapy. For suspected low-grade oligodendrogliomas in select patients, some neuro-oncologists opt for a course of watchful waiting, with only symptomatic therapy. Tumors with the 1p/19q co-deletion have been found to be especially chemosensitive, and one source reports oligodendrogliomas to be among the most chemosensitive of human solid malignancies. A median survival of up to 16.7 years has been reported for low grade oligodendrogliomas.

Although there is no specific or singular clinical symptom or sign for any brain tumors, the presence of a combination of symptoms and the lack of corresponding clinical indications of infections or other causes can be an indicator to redirect diagnostic investigation towards the possibility of an intracranial neoplasm.

The diagnosis will often start with an interrogation of the patient to get a clear view of his medical antecedents, and his current symptoms. Clinical and laboratory investigations will serve to exclude infections as the cause of the symptoms. Examinations in this stage may include ophthalmological, otolaryngological (or ENT) and/or electrophysiological exams. The use of electroencephalography (EEG) often plays a role in the diagnosis of brain tumors.

Swelling, or obstruction of the passage of cerebrospinal fluid (CSF) from the brain may cause (early) signs of increased intracranial pressure which translates clinically into headaches, vomiting, or an altered state of consciousness, and in children changes to the diameter of the skull and bulging of the fontanelles. More complex symptoms such as endocrine dysfunctions should alarm doctors not to exclude brain tumors.

A bilateral temporal visual field defect (due to compression of the optic chiasm) or dilatation of the pupil, and the occurrence of either slowly evolving or the sudden onset of focal neurologic symptoms, such as cognitive and behavioral impairment (including impaired judgment, memory loss, lack of recognition, spatial orientation disorders), personality or emotional changes, hemiparesis, hypoesthesia, aphasia, ataxia, visual field impairment, impaired sense of smell, impaired hearing, facial paralysis, double vision, or more severe symptoms such as tremors, paralysis on one side of the body hemiplegia, or (epileptic) seizures in a patient with a negative history for epilepsy, should raise the possibility of a brain tumor.

Imaging plays a central role in the diagnosis of brain tumors. Early imaging methods—invasive and sometimes dangerous—such as pneumoencephalography and cerebral angiography, have been abandoned in recent times in favor of non-invasive, high-resolution techniques, such as computed tomography (CT)-scans and especially magnetic resonance imaging (MRI). Neoplasms will often show as differently colored masses (also referred to as processes) in CT or MRI results.

Benign brain tumors often show up as hypodense (darker than brain tissue) mass lesions on cranial CT-scans. On MRI, they appear either hypo- (darker than brain tissue) or isointense (same intensity as brain tissue) on T1-weighted scans, or hyperintense (brighter than brain tissue) on T2-weighted MRI, although the appearance is variable.

Contrast agent uptake, sometimes in characteristic patterns, can be demonstrated on either CT or MRI-scans in most malignant primary and metastatic brain tumors. Perifocal edema, or pressure-areas, or where the brain tissue has been compressed by an invasive process also appears hyperintense on T2-weighted MRI might indicate the presence a diffuse neoplasm (unclear outline). This is because these tumors disrupt the normal functioning of the blood-brain barrier and lead to an increase in its permeability. However, it is not possible to diagnose high versus low grade gliomas based on enhancement pattern alone.

Glioblastoma multiforme and anaplastic astrocytoma have been associated with the genetic acute hepatic porphyrias (PCT, AIP, HCP and VP), including positive testing associated with drug refractory seizures. Unexplained complications associated with drug treatments with these tumors should alert physicians to an undiagnosed neurological porphyria.

The definitive diagnosis of brain tumor can only be confirmed by histological examination of tumor tissue samples obtained either by means of brain biopsy or open surgery. The histological examination is essential for determining the appropriate treatment and the correct prognosis. This examination, performed by a pathologist, typically has three stages: interoperative examination of fresh tissue, preliminary microscopic examination of prepared tissues, and followup examination of prepared tissues after immunohistochemical staining or genetic analysis.

When a brain tumor is diagnosed, a medical team will be formed to assess the treatment options presented by the leading surgeon to the patient and his/her family. Given the location of primary solid neoplasms of the brain in most cases a “do-nothing” option is usually not presented. Neurosurgeons take the time to observe the evolution of the neoplasm before proposing a management plan to the patient and his/her relatives. These various types of treatment are available depending on neoplasm type and location and may be combined to give the best chances of survival: surgery: complete or partial resection of the tumor with the objective of removing as many tumor cells as possible; radiotherapy; and chemotherapy, with the aim of killing as many as possible of cancerous cells left behind after surgery and of putting remaining tumor cells into a nondividing, sleeping state for as long as possible.

Survival rates in primary brain tumors depend on the type of tumor, age, functional status of the patient, the extent of surgical tumor removal and other factors specific to each case.

The primary and most desired course of action described in medical literature is surgical removal (resection) via craniotomy. Minimally invasive techniques are being studied but are far from being common practice. The prime remediating objective of surgery is to remove as many tumor cells as possible, with complete removal being the best outcome and cytoreduction (“debulking”) of the tumor otherwise. In some cases access to the tumor is impossible and impedes or prohibits surgery.

Many meningiomas, with the exception of some tumors located at the skull base, can be successfully removed surgically. Most pituitary adenomas can be removed surgically, often using a minimally invasive approach through the nasal cavity and skull base (trans-nasal, trans-sphenoidal approach). Large pituitary adenomas require a craniotomy (opening of the skull) for their removal. Radiotherapy, including stereotactic approaches, is reserved for inoperable cases.

Several current research studies aim to improve the surgical removal of brain tumors by labeling tumor cells with a chemical (5-aminolevulinic acid) that causes them to fluoresce. Post-operative radiotherapy and chemotherapy are integral parts of the therapeutic standard for malignant tumors. Radiotherapy may also be administered in cases of “low-grade” gliomas, when a significant tumor burden reduction could not be achieved surgically.

Any person undergoing brain surgery may suffer from epileptic seizures. Seizures can vary from absences to severe tonic-clonic attacks. Medication is prescribed and administered to minimize or eliminate the occurrence of seizures.

Multiple metastatic tumors are generally treated with radiotherapy and chemotherapy rather than surgery, the prognosis in such cases is determined by the primary tumor, but is generally poor.

The goal of radiation therapy is to selectively kill tumor cells while leaving normal brain tissue unharmed. In standard external beam radiation therapy, multiple treatments of standard-dose “fractions” of radiation are applied to the brain. This process is repeated for a total of 10 to 30 treatments, depending on the type of tumor. This additional treatment provides some patients with improved outcomes and longer survival rates.

Radiosurgery is a treatment method that uses computerized calculations to focus radiation at the site of the tumor while minimizing the radiation dose to the surrounding brain. Radiosurgery may be an adjunct to other treatments, or it may represent the primary treatment technique for some tumors.

Radiotherapy may be used following, or in some cases in place of, resection of the tumor. Forms of radiotherapy used for brain cancer include external beam radiation therapy, brachytherapy, and in more difficult cases, stereotactic radiosurgery, such as Gamma knife, Cyberknife or Novalis Tx radiosurgery.

Radiotherapy is the most common treatment for secondary brain tumors. The amount of radiotherapy depends on the size of the area of the brain affected by cancer. Conventional external beam ‘whole brain radiotherapy treatment’ (WBRT) or ‘whole brain irradiation’ may be suggested if there is a risk that other secondary tumors will develop in the future. Stereotactic radiotherapy is usually recommended in cases involving fewer than three small secondary brain tumors.

Patients undergoing chemotherapy are administered drugs designed to kill tumor cells. Although chemotherapy may improve overall survival in patients with the most malignant primary brain tumors, it does so in only about 20 percent of patients. Chemotherapy is often used in young children instead of radiation, as radiation may have negative effects on the developing brain. The decision to prescribe this treatment is based on a patient's overall health, type of tumor, and extent of the cancer. The toxicity and many side effects of the drugs, and the uncertain outcome of chemotherapy in brain tumors puts this treatment further down the line of treatment options with surgery and radiation therapy preferred.

A shunt is used not as a cure but to relieve symptoms by reducing hydrocephalus caused by blockage of cerebrospinal fluid.

Researchers are presently investigating a number of promising new treatments including gene therapy, highly focused radiation therapy, immunotherapy and novel chemotherapies. A variety of new treatments are being made available on an investigational basis at centers specializing in brain tumor therapies.

### Pancreatic Cancer

Pancreatic cancer arises when cells in the pancreas, a glandular organ behind the stomach, begin to multiply out of control and form a mass. These cancerous cells have the ability to invade other parts of the body. A number of types of pancreatic cancer are known.

The most common, pancreatic adenocarcinoma, accounts for about 90% of cases, and the term “pancreatic cancer” is sometimes used to refer only to that type. These adenocarcinomas start within the part of the pancreas that makes digestive enzymes. Several other types of cancer, which collectively represent the majority of the non-adenocarcinomas, can also arise from these cells. About 1-2% of cases of pancreatic cancer are neuroendocrine tumors, which arise from the hormone-producing cells of the pancreas. These are generally less aggressive than pancreatic adenocarcinoma.

Signs and symptoms of the most-common form of pancreatic cancer may include yellow skin, abdominal or back pain, unexplained weight loss, light-colored stools, dark urine, and loss of appetite. Usually, no symptoms are seen in the disease's early stages, and symptoms that are specific enough to suggest pancreatic cancer typically do not develop until the disease has reached an advanced stage. By the time of diagnosis, pancreatic cancer has often spread to other parts of the body.

Pancreatic cancer rarely occurs before the age of 40, and more than half of cases of pancreatic adenocarcinoma occur in those over 70. Risk factors for pancreatic cancer include tobacco smoking, obesity, diabetes, and certain rare genetic conditions. About 25% of cases are linked to smoking, and 5-10% are linked to inherited genes. Pancreatic cancer is usually diagnosed by a combination of medical imaging techniques such as ultrasound or computed tomography, blood tests, and examination of tissue samples (biopsy). The disease is divided into stages, from early (stage I) to late (stage IV). Screening the general population has not been found to be effective.

The risk of developing pancreatic cancer is lower among nonsmokers, and people who maintain a healthy weight and limit their consumption of red or processed meat. Smokers' chances of developing the disease decrease if they stop smoking and almost return to that of the rest of the population after 20 years. Pancreatic cancer can be treated with surgery, radiotherapy, chemotherapy, palliative care, or a combination of these. Treatment options are partly based on the cancer stage. Surgery is the only treatment that can cure pancreatic adenocarcinoma, and may also be done to improve quality of life without the potential for cure. Pain management and medications to improve digestion are sometimes needed. Early palliative care is recommended even for those receiving treatment that aims for a cure.

In 2015, pancreatic cancers of all types resulted in 411,600 deaths globally. Pancreatic cancer is the fifth-most-common cause of death from cancer in the United Kingdom, and the third most-common in the United States. The disease occurs most often in the developed world, where about 70% of the new cases in 2012 originated. Pancreatic adenocarcinoma typically has a very poor prognosis; after diagnosis, 25% of people survive one year and 5% live for five years. For cancers diagnosed early, the five-year survival rate rises to about 20%. Neuroendocrine cancers have better outcomes; at five years from diagnosis, 65% of those diagnosed are living, though survival considerably varies depending on the type of tumor.

The many types of pancreatic cancer can be divided into two general groups. The vast majority of cases (about 95%) occur in the part of the pancreas that produces digestive enzymes, known as the exocrine component. Several subtypes of exocrine pancreatic cancers are described, but their diagnosis and treatment have much in common. The small minority of cancers that arise in the hormone-producing (endocrine) tissue of the pancreas have different clinical characteristics and are called pancreatic neuroendocrine tumors, sometimes abbreviated as “PanNETs”. Both groups occur mainly (but not exclusively) in people over 40, and are slightly more common in men, but some rare subtypes mainly occur in women or children.

Since pancreatic cancer usually does not cause recognizable symptoms in its early stages, the disease is typically not diagnosed until it has spread beyond the pancreas itself. This is one of the main reasons for the generally poor survival rates. Exceptions to this are the functioning PanNETs, where over-production of various active hormones can give rise to symptoms (which depend on the type of hormone).

Bearing in mind that the disease is rarely diagnosed before the age of 40, common symptoms of pancreatic adenocarcinoma occurring before diagnosis include:

Pain in the upper abdomen or back, often spreading from around the stomach to the back. The location of the pain can indicate the part of the pancreas where a tumor is located. The pain may be worse at night and may increase over time to become severe and unremitting. It may be slightly relieved by bending forward. In the UK, about half of new cases of pancreatic cancer are diagnosed following a visit to a hospital emergency department for pain or jaundice. In up to two-thirds of people, abdominal pain is the main symptom, for 46% of the total accompanied by jaundice, with 13% having jaundice without pain.

Jaundice, a yellow tint to the whites of the eyes or skin, with or without pain, and possibly in combination with darkened urine, results when a cancer in the head of the pancreas obstructs the common bile duct as it runs through the pancreas.

Unexplained weight loss, either from loss of appetite, or loss of exocrine function resulting in poor digestion.

The tumor may compress neighboring organs, disrupting digestive processes and making it difficult for the stomach to empty, which may cause nausea and a feeling of fullness. The undigested fat leads to foul-smelling, fatty feces that are difficult to flush away. Constipation is also common.

At least 50% of people with pancreatic adenocarcinoma have diabetes at the time of diagnosis. While long-standing diabetes is a known risk factor for pancreatic cancer (see Risk factors), the cancer can itself cause diabetes, in which case recent onset of diabetes could be considered an early sign of the disease. People over 50 who develop diabetes have eight times the usual risk of developing pancreatic adenocarcinoma within three years, after which the relative risk declines.

A key assessment that is made after diagnosis is whether surgical removal of the tumor is possible (see Staging), as this is the only cure for this cancer. Whether or not surgical resection can be offered depends on how much the cancer has spread. The exact location of the tumor is also a significant factor, and CT can show how it relates to the major blood vessels passing close to the pancreas. The general health of the person must also be assessed, though age in itself is not an obstacle to surgery.

Chemotherapy and, to a lesser extent, radiotherapy are likely to be offered to most people, whether or not surgery is possible. Specialists advise that the management of pancreatic cancer should be in the hands of a multidisciplinary team including specialists in several aspects of oncology, and is, therefore, best conducted in larger centers.

Treatment of PanNETs, including the less common malignant types, may include a number of approaches. Some small tumors of less than 1 cm. that are identified incidentally, for example on a CT scan performed for other purposes, may be followed by watchful waiting. This depends on the assessed risk of surgery which is influenced by the site of the tumor and the presence of other medical problems. Tumors within the pancreas only (localized tumors), or with limited metastases, for example to the liver, may be removed by surgery. The type of surgery depends on the tumor location, and the degree of spread to lymph nodes.

For localized tumors, the surgical procedure may be much less extensive than the types of surgery used to treat pancreatic adenocarcinoma described above, but otherwise surgical procedures are similar to those for exocrine tumors. The range of possible outcomes varies greatly; some types have a very high survival rate after surgery while others have a poor outlook. As all this group are rare, guidelines emphasize that treatment should be undertaken in a specialized center. Use of liver transplantation may be considered in certain cases of liver metastasis.

For functioning tumors, the somatostatin analog class of medications, such as octreotide, can reduce the excessive production of hormones. Lanreotide can slow tumor growth. If the tumor is not amenable to surgical removal and is causing symptoms, targeted therapy with everolimus or sunitinib can reduce symptoms and slow progression of the disease. Standard cytotoxic chemotherapy is generally not very effective for PanNETs, but may be used when other drug treatments fail to prevent the disease from progressing, or in poorly differentiated PanNET cancers.

Radiation therapy is occasionally used if there is pain due to anatomic extension, such as metastasis to bone. Some PanNETs absorb specific peptides or hormones, and these PanNETs may respond to nuclear medicine therapy with radiolabeled peptides or hormones such as iobenguane (iodine-131-MIBG). Radiofrequency ablation (RFA), cryoablation, and hepatic artery embolization may also be used.

### Methods of Diagnosis

The present disclosure further has a diagnostic component.

More specifically, an aspect of the present disclosure is a method for determining if a subject is treatable, e.g., would get a therapeutic benefit, by a composition comprising a compound of Formula I:

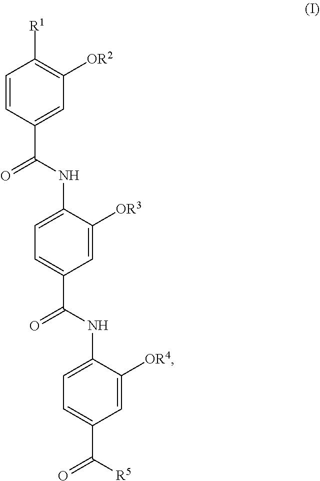

the method comprising testing a sample from the subject for the presence of elevated Lysosomal lipase A (LIPA) expression and when the sample indicates the presence of elevated LIPA or increased activity of LIPA, the subject is treatable by a composition comprising a compound of Formula L.

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In various embodiments, R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein: R^{1a} is aralkyl_{(C<18)}, substituted aralkyl_{(C<18)}, or the side chain of a canonical amino acid; R², R³, and R⁴ are each independently alkyl_{(C<12)}, substituted alkyl_{(C<12)}, aralkyl_{(C<18)}, or substituted aralkyl_{(C<18)}; and R⁵ is —OR^{5a} or —NHR^{5b}, wherein: R^{5a} is alkyl_{(C<12)} or substituted alkyl_{(C<12)}; R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or a group of the formula:

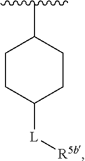

wherein; L is —CO₂— or —C(O)NR^{L}—, wherein: R^{L} hydrogen, alkyl_{(C<12)}, or substituted alkyl_{(C<12)}; R^{5b′} is aryl_{(C<12)}, aralkyl_{(C<18)}, heteroaryl_{(C<12)}, heteroaralkyl_{(C<18)}, or a substituted version of any of these groups.

In various embodiments, the method further comprises administering a therapeutically-effective amount of the composition.

In some embodiments, the compound is a pharmaceutically acceptable salt of Formula I.

In several embodiments, the compound of Formula I inhibits LIPA activity when contacted to the LXXLL motif on LIPA.

In embodiments, the compound or composition is administered in an amount sufficient to induce endoplasmic reticulum stress, in an amount sufficient to inhibit activity of LIPA, and/or in an amount sufficient to reduce protein synthesis.

An additional aspect of the present disclosure is a method for determining if a subject is treatable, e.g., would get a therapeutic benefit, by a composition comprising the compound of Formula II:

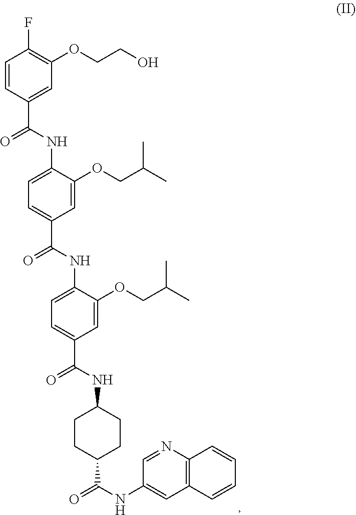 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

the method comprising testing a sample from the subject for the presence of elevated Lysosomal lipase A (LIPA) expression and when the sample indicates the presence of elevated LIPA or increased activity of LIPA, the subject is treatable by a composition comprising the compound of Formula II.

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In various embodiments, the method further comprises administering a therapeutically-effective amount of the composition.

In some embodiments, the compound is a pharmaceutically acceptable salt of Formula II.

In several embodiments, the compound of Formula II inhibits LIPA activity when contacted to the LXXLL motif on LIPA.

In embodiments, the compound or composition is administered in an amount sufficient to induce endoplasmic reticulum stress, in an amount sufficient to inhibit activity of LIPA, and/or in an amount sufficient to reduce protein synthesis.

### Methods of Synthesis

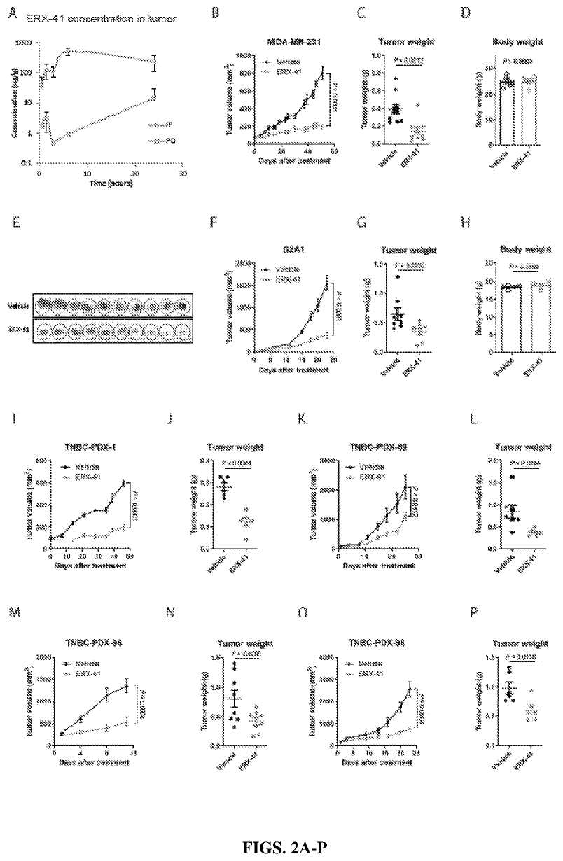

U.S. Patent Publication 2009/0012141 provides synthesis schemes to prepare α-helix mimetic compounds of the present disclosure, for example, in FIG. 2 therein. A specific example in that document provides fifteen α-helix mimetic compounds made starting with a 4-amino-3-hydroxybenzoic acid compound 7, which was converted to an N—Ac protected methyl ester compound 8. Various alkyl groups were introduced to the hydroxyl group using a variety of alkyl halides and a base (e.g., NaOH) known to the skilled artisan. After the alkylation reaction, the methyl ester compound 9 was hydrolyzed using a base (like LiOH), and methyl 4-amino-3-hydroxybenzoate compound 10 was coupled to the free benzoic acid using a coupling reagent (like BOP), resulting in a benzamide compound 11 containing one alkyl group corresponding to the i position of a helix. These steps were repeated to synthesize oligo-benzamide compounds. Those of skill in the art would understand the broader applicability of such methods in the synthesis of other compounds such as those disclosed herein.

Additional peptidomimetics as well as methods for their manufacture are disclosed in Raj et al., 2017, which is incorporated herein by reference. One of skill in the art appreciates that the synthetic methods disclosed in Raj et al. (2017) may be employed to construct the compounds of the present disclosure. The compounds of the present disclosure may also be made using the synthetic methods outlined in the Examples section and/or as described in WO2020117715A1, the contents of which is incorporated herein by reference in its entirety. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

Methods of synthesis are further descried in the below Examples.

### Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “----” represents an optional bond, which if present is either single or double. The symbol “ [symbol] ” resents a single bond or a double bond. Thus, the formula

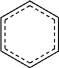

covers, for example,

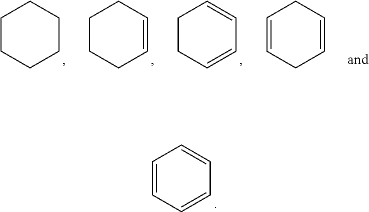

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

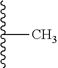

for methyl) indicates a point of attachment of the group. It is noted that the point of attachment is typically only identified in this manner for larger groups in order to assist the reader in unambiguously identifying a point of attachment. The symbol “ [symbol] ” means single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

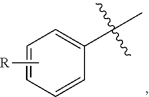

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

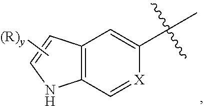

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question.

For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. Except as noted below, every carbon atom is counted to determine whether the group or compound falls with the specified number of carbon atoms. For example, the group dihexylamino is an example of a dialkylamino_{(C=12)} group; however, it is not an example of a dialkylamino_{(C=6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)} Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

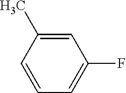

is also taken to refer to

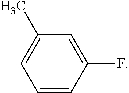

Aromatic compounds may also be depicted using a circle to represent the delocalized nature of the electrons in the fully conjugated cyclic n system, two non-limiting examples of which are shown below:

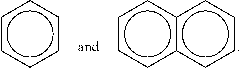

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

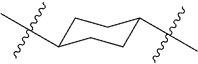 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

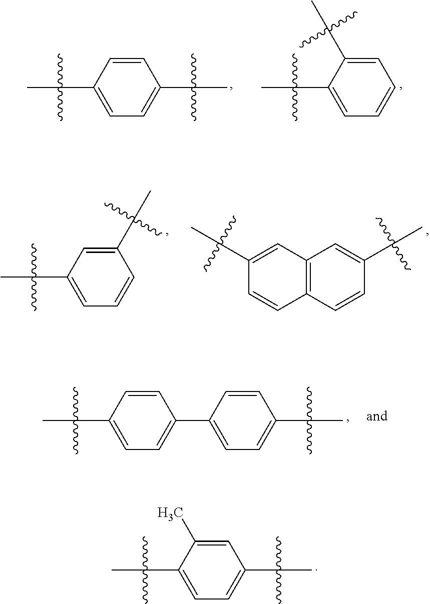

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn),2-phenyl-ethyl, and (naphthalen-2-yl)methyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl (Im), isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl. The term “amido” (acylamino), when used without the “substituted” modifier, refers to the group —NHR, in which R is acyl, as that term is defined above. A non-limiting example of an amido group is —NHC(O)CH₃ or NHC(O)C₆H₁₁.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e., —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, 4-hydroxyphenethyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃, —NHC(O)NHCH₃, and —NHC(O)C₆H₁₀NH₂, are non-limiting examples of substituted amido groups.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and claims, the singular forms “a”, “an” and “the” include plural references unless the context clearly dictates otherwise.

The use of the word “a” or “an,” when used in conjunction with the term “comprising” in the claims and/or the specification may mean “one,” but it is also consistent with the meaning of “one or more,” “at least one,” and “one or more than one.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. The skilled artisan will understand that typically there is no limit on the number of items or terms in any combination, unless otherwise apparent from the context.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e., an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

The section headings used herein are for organizational purposes only and are not to be construed as limiting the subject matter described.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials & Methods

Chemicals and synthetic procedure: All chemical reagents and solvents were obtained from commercial sources and used without additional purification. ¹H and ¹³C nuclear magnetic resonance (NMR) spectra were recorded on a Bruker Avance III HD 600 MHz NMR spectrometer. Chemical shifts are reported in parts per million (δ) from an internal standard of residual DMSO-d6 (2.50 and 39.5 ppm in ¹H and ¹³C-NMR spectra, respectively). Data are reported as follows: chemical shift (δ), multiplicity (s, singlet; d, doublet; dd, doublet of doublet; t, triplet; q, quartet; br s, broad singlet; m, multiplet), coupling constant (J) in Hertz (Hz), integration. Mass spectra (MS) were recorded on a Shimadzu AXIMA Confidence MALDI-TOF mass spectrometer (nitrogen UV laser, 50 Hz, 337 nm) by using α-cyano-4-hydroxycinnamic acid (CHCA) as a matrix.

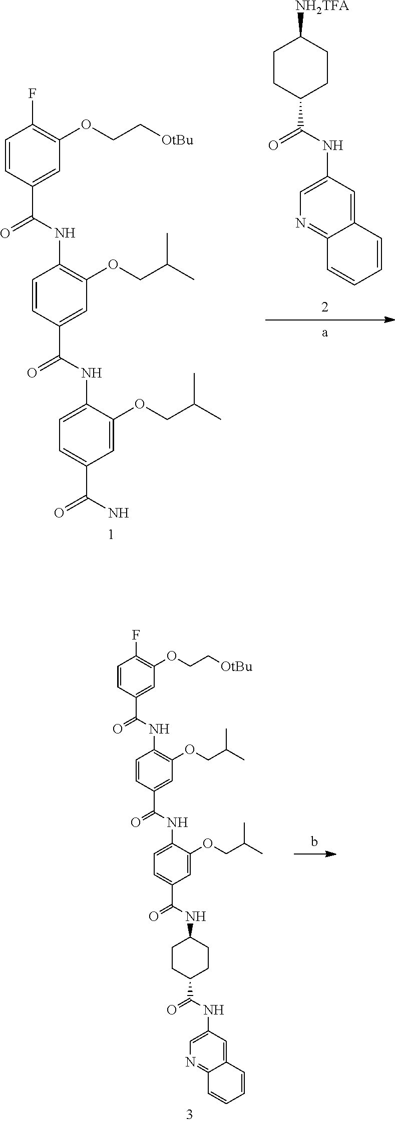

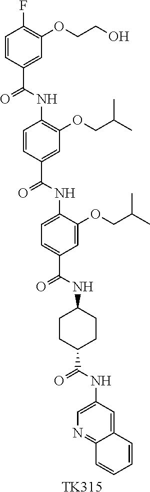 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1 ; "TK315"]

A solution of compound 1 (shown above, 50 mg, 0.078 mmol), HATU (39 mg, 0.10 mmol), DIEA (41 pL, 0.24 mmol) in DMF (4 mL) was stirred at room temperature for 1 h, and then compound 2 (shown above, 91 mg, 0.24 mmol) was added to the reaction mixture. The resulting mixture was stirred at room temperature for 24 h and then diluted with EtOAc (20 mL) and 1 N HCl (10 mL). The organic layer was separated, washed with 1 N HCl (10 mL) and brine (10 mL), and concentrated under reduced pressure. The resulting solid was washed with EtOAc and dried in vacuo to give compound 3 as a yellow sold.

A solution of compound 3 (shown above) in TFA (3 mL) was stirred at room temperature for 1 h and then concentrated under reduced pressure. The resulting solid was washed with ether and dried in vacuo to give ERX-315 as a yellow solid (51 mg, 78% over 2 reaction steps).

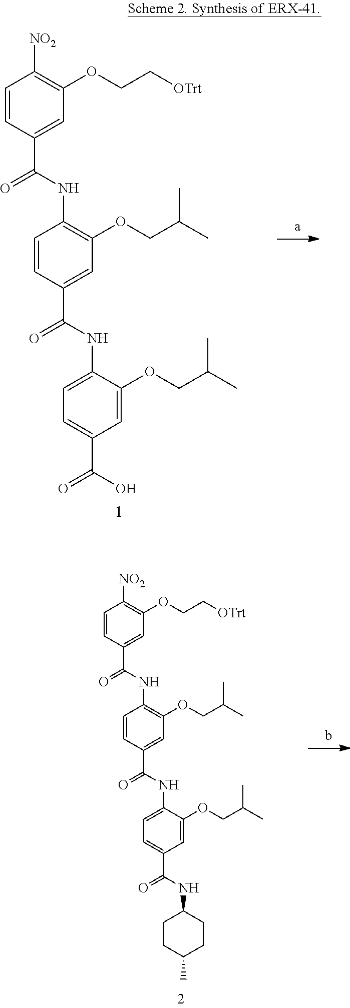

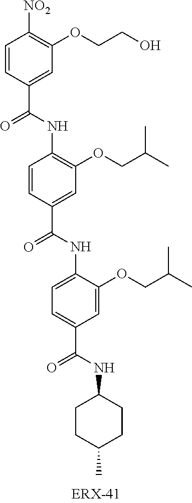 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

Reagents and conditions: (a) trans-4-methylcyclohexylamine, HATU, DIEA, DMF, rt, 24 h; (b) conc. HCl, THF, rt, 24 h. Synthesis of compound 2: The compound 1 (shown above) was synthesized as previously reported (McInerney, E. M. et al. Determinants of coactivator LXXLL motif specificity in nuclear receptor transcriptional activation. Genes Dev 12, 3357-3368 (1998)). Trans-4-methylcyclohexylamine (0.73 g, 6.4 mmol) was added to a solution of compound 1 (2.7 g, 3.2 mmol), HATU (1.4 g, 3.7 mmol), DIEA (1.2 mL, 6.9 mmol) and DMF (30 mL). The reaction mixture was stirred at room temperature for 24 h and then diluted with EtOAc (100 mL) and 0.5 N HCl (100 mL). The layers were separated, and the aqueous layer was extracted with EtOAc (100 mL). The organic layers were combined, washed with 0.5 N HCl and brine, dried over anhydrous sodium sulfate, and concentrated under reduced pressure. The resulting solid was washed with EtOAc and dried in vacuo to give compound 2 (shown above) as a white sold (1.75 g). The product was used in the next reaction without further purification.

Synthesis of ERX-41: Concentrated HCl (30 mL) was added to a solution of compound 2 (1.75 g) and THF (300 mL). The reaction mixture was stirred at room temperature for 24 h and then concentrated under reduced pressure. The resulting solid was washed with MeOH and dried in vacuo to give ERX-41 as a light-yellow solid (1.3 g, 57% over 2 reaction steps). ¹H NMR (DMSO-d₆, 600 MHz): δ 9.85 (br s, 1H), 9.45 (br s, 1H), 8.15 (d, J=7.7 Hz, 1H), 8.02 (d, J=8.4 Hz, 1H), 7.96 (d, J=8.6 Hz, 1H), 7.92 (d, J=8.2 Hz, 1H), 7.85 (s, 1H), 7.64 (s, 1H), 7.63 (d, J=8.2 Hz, 1H), 7.60 (d, J=8.4 Hz, 1H), 7.52 (br s, 1H), 7.51 (d, J=6.2 Hz, 1H), 4.96 (t, J=5.2 Hz, 1H), 4.29 (t, J=5.0 Hz, 2H), 3.91 (d, J=5.9 Hz, 2H), 3.90 (d, J=5.9 Hz, 2H), 3.76 (q, J=5.1 Hz, 2H), 3.75-3.71 (m, 1H), 2.15-2.07 (m, 2H), 1.84 (d, J=12.4 Hz, 2H), 1.71 (d, J=12.5 Hz, 2H), 1.40-1.31 (m, 3H), 1.05-1.03 (m, 1H, overlapped with the signal of CH(CH₃)₂), 1.03 (d, J=6.6 Hz, 6H), 1.01-1.00 (m, 1H, overlapped with the signal of CH(CH₃)₂), 1.01 (d, J=6.6 Hz, 6H), 0.90 (d, J=6.6 Hz, 3H). ¹³C NMR (DMSO-d₆, 150 MHz): δ 164.7, 164.2, 163.6, 151.1, 150.8, 149.8, 141.4, 139.2, 131.9, 131.6, 129.8, 129.6, 125.0, 124.0, 122.2, 119.73, 119.66, 119.59, 114.2, 111.1, 110.9, 74.6, 74.5, 71.5, 59.2, 48.4, 33.8, 32.3, 31.6, 27.9, 27.8, 22.2, 19.13, 19.05. MALDI-TOF (m/z): [M+Na]⁺ calculated for C₃₈H₄₈N₄NaO₉: 727.2, found 727.6.

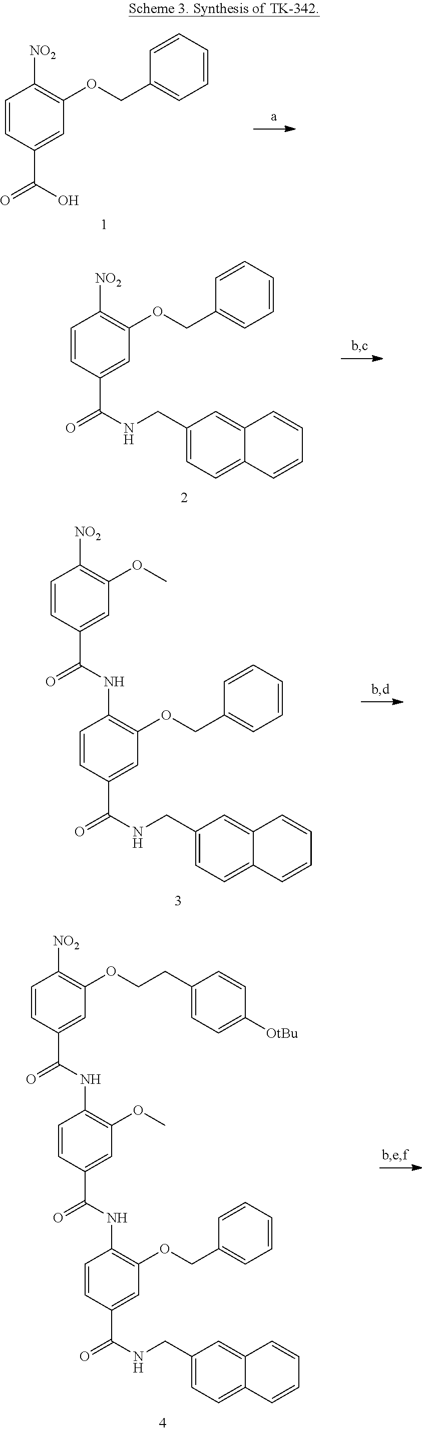 [SMILES: O=C(O)c1ccc([N+](=O)[O-])c(OCc2ccccc2)c1 ; O=C(NCc1ccc2ccccc2c1)c1ccc([N+](=O)[O-])c(OCc2ccccc2)c1 ; COc1cc(C(=O)Nc2ccc(C(=O)NCc3ccc4ccccc4c3)cc2OCc2ccccc2)ccc1[N+](=O)[O-] ; COc1cc(C(=O)Nc2ccc(C(=O)NCc3ccc4ccccc4c3)cc2OCc2ccccc2)ccc1NC(=O)c1ccc([N+](=O)[O-])c(OCCc2ccc(OC(C)(C)C)cc2)c1]

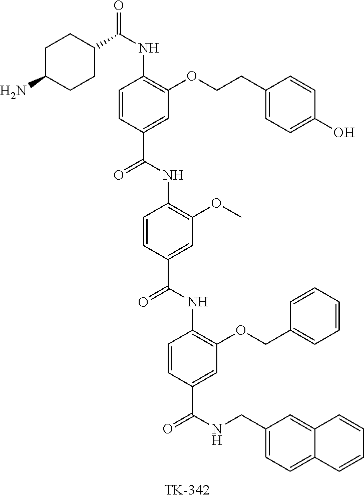

Reagents and conditions: (a) 2-naphthylmethylamine, HATU; (b) Fe/AcOH; (c) 4-nitro-3-methoxybenzoic acid, HATU; (d) 4-nitro-3-(p-t-butoxyphenylethoxy)benzoic acid, HATU; (e) Boc-aminocyclohexanecarboxylic acid, HATU; (f) TFA. Synthesis of compound 2: 2-naphthylmethylamine and Hexafluorophosphate Azabenzotriazole Tetramethyl Uronium (HATU), and DIEA (“Hünig's base”) were added to a round bottom flask containing a solution of compound 1 (shown above) and DMF, however other polar aprotic solvents may be used. The resulting mixture was stirred under conditions sufficient to provide compound 2 (e.g., rt for 24 hrs). Compound 2 was then isolated and purified using standard workup conditions. See, for example, the workup conditions provided in Schemes 1 and 2.

Synthesis of compound 3: iron (Fe) and acetic acid (AcOH) were added to a round bottom flask containing a solution comprising compound 2. The resulting mixture was heated and stirred under conditions sufficient to reduce the nitrobenzene group of compound 2 to provide the aniline analog (e.g., via Béchamp reduction). The analine analog may be isolated, and subsequently 4-nitro-3-methoxybenzoic acid, HATU, and DIEA were added to a round bottom flask containing a solution comprising the reduced form of compound 2 (i.e., the analine analog) and DMF, however other polar aprotic solvents may be used. The resulting mixture was stirred under conditions sufficient to provide compound 3 (e.g., rt for 24 hrs). Compound 3 was isolated and purified using standard workup conditions. See, for example, the workup conditions provided in Schemes 1 and 2.

Synthesis of compound 4: iron (Fe) and acetic acid (AcOH) were added to a round bottom flask containing a solution comprising compound 3. The resulting mixture was heated and stirred under conditions sufficient to reduce the nitrobenzene group of compound 3 to provide the aniline analog (e.g., via Béchamp reduction). The analine analog may be isolated, and subsequently 4-nitro-3-(p-t-butoxyphenylethoxy)benzoic acid, HATU, and DIEA were added to a round bottom flask containing a solution comprising the reduced form compound 3 (i.e., the analine analog) and DMF, however other polar aprotic solvents may be used. The resulting mixture was stirred under conditions sufficient to provide compound 4 (e.g., rt for 24 hrs). Compound 4 was isolated and purified using standard workup conditions. See, for example, the workup conditions provided in Schemes 1 and 2.

Synthesis of compound TK-342: iron (Fe) and acetic acid (AcOH) were added to a round bottom flask containing a solution comprising compound 4. The resulting mixture was heated and stirred under conditions sufficient to reduce the nitrobenzene group of compound 4 to provide the aniline analog (e.g., via Béchamp reduction). The analine analog may be isolated, and subsequently Boc-aminocyclohexanecarboxylic acid, HATU, and DIEA were added to a round bottom flask containing a solution comprising the reduced form compound 4 (i.e., the analine analog) and DMF, however other polar aprotic solvents may be used. The resulting mixture was stirred under conditions sufficient to provide the protected form of compound TK-342 (e.g., rt for 24 hrs). Compound TK-342 is deprotected using trifluoroacetic acid (TFA). Compound TK-342 was isolated and purified using standard workup conditions. See, for example, the workup conditions provided in Schemes 1 and 2.

Resulting solids were collected and dried in vacuo to give TK-342 shown above. Samples were analyzed using ¹H NMR (DMSO-d₆, 600 MHz) to confirm purity.

Molecular Docking Study: AutoDock Tools 1.5.6 (ADT; The Scripps Research Institute, La Jolla, CA, USA, RRID:SCR_012746) was used to create input PDBQT files of a protein and a ligand. The input file of human lysosomal acid lipase (LAL) was prepared using the published coordinates (PDB code: 6V7N). Water molecules were removed from the protein structure, and hydrogens were added. All other atom values were generated automatically by ADT. The grid box was centered on the helical motif (²³⁸NLCFLLC²⁴⁴) and to accommodate ligand to move freely. The grid box was set to 35×35×35 Å, and the x, y, and z coordinates of the center of the grid box were set to 123, 29, and 140, respectively. The input file of ERX-41 was created from its energy-minimized conformation using ADT. Docking calculation was performed with AutoDock Vina 1.1.2 [2]. A search exhaustiveness of 16 was used and all other parameters were left as default values. A predicted binding mode was visualized using Maestro (version 9.1, Schrödinger, LLC, New York, NY, USA, 2010, RRID:SCR_016748).

Cell Culture: Human breast cancer cells MCF-7, ZR-75, T-47D, MDA-MB-231, BT474, BT549, BT453, SUM-159, 4T1, MM468, HCC1937, HCC1187 (ATCC Cat #CRL-2322, RRID:CVCL_1247), HCC70 (BCRJ Cat #0386, RRID:CVCL_1270), MDA-MB-157 (ATCC Cat #CRL-7721, RRID:CVCL_0618), MDA-MB-453, MDA-MB-468 (NCI-DTP Cat #MDA-MB-468, RRID:CVCL_0419) were either obtained from American Type Culture Collection (ATCC, Manassas, VA) or from UT Southwestern core and cultured per ATCC guidelines. All the model cells utilized were free of Mycoplasma contamination. STR DNA profiling was used to confirm cell identity. BT-20 (ATCC #HTB-19), BT549 (ATCC #HTB-122), ES2 (ATCC #CRL-1978), HCC38 (ATCC Cat #CRL-2314, RRID:CVCL_1267), HCC70, HCC202 (ATCC Cat #CRL-2316, RRID:CVCL_2062), HCC1143 (ATCC Cat #CRL-2321, RRID:CVCL_1245), HCC1187, HCC1419 (KCLB Cat #9S1419, RRID:CVCL_1251 (link), HCC1569 (KCLB Cat #71569, RRID:CVCL_1255), HCC1806 (KCB Cat #KCB 2014032YJ, RRID:CVCL_1258), HCC1937, HCC1954, HCC2185 (ATCC Cat #CRL-2342, RRID:CVCL_3375), HCC2688 (RRID:CVCL_3376), MDA-MB-157, MDA-MB-231, MDA-MB-436, MDA-MB-453, MDA-MB-468, SKBR3 and UACC812 cells were cultured in RPMI1640 (Sigma) supplemented with 10% FBS, 0.2% Normocin and 1% penicillin-streptomycin. SUM-159 cells were cultured in F-12 (Gibco) supplemented with 10% FBS, 0.2% Normocin and 1% penicillin-streptomycin. HMEC cells were cultured in HuMEC Ready media (Invitrogen) supplemented with 0.2% Normocin and 1% penicillin-streptomycin.

Estrogen receptor binding: The LanthaScreen TR-FRET ER alpha Coactivator Assay (Kit #Catalog no. A15885, life technologies) was performed per manufacturer's instructions. The compounds were tested in the range of 0.0003-16.66 μM using serial dilutions and the assays was done in antagonist assay mode. The final assay buffer composition included 3.5 nM ER Alpha-LBD (GST), 250 nM Fluorescein-conjugated coactivator PGC1a peptide, 5 nM terbium (Tb)-labeled anti-GST antibody, and 5 nM estradiol. The plate was incubated at room temperature for 2 h and FRET was analyzed on PHERAstar microplate reader with the following setting: Excitation: 340 nm, Emission: 495 nm and 520 nm. The emission ratio (520:495) was analyzed and plotted. Curves were generated using a sigmoidal dose response equation (variable slope) in GraphPad Prism™ 9.0 software.

Cell viability assays: Cells were seeded in 96-well plates (2×10³ cells/well) one day before the treatment. Cells were treated with varying concentrations of ERX-41 or ERX-11 analogs for 3-6 days. Then effects of ERX-41 or ERX-11 analogs on cell viability were measured in triplicate or sextuplicate with multiple biological replicates using the WST-1 (Promega), MTT as previously described³³ or CellTiter-Glo 2.0 (Promega) assays.

Live cell imaging: Live cell images were acquired by Lionheart FX automated microscope (BioTek). Cells were plated one day before the experiment. For cell death assay, 0.2 μM SYTOX Green was added into the plate 15 min before the experiment.

CRISPR Screen: Human Brunello CRISPR knockout pooled library was purchased from Addgene (Addgene #73178) (via David Root and John Doench). The library containing lentivirus was transduced into cells in biological replicates or triplicates at a multiplicity of infection (MOI) of ˜0.5 and a minimum of 500× coverage. Two days later after transduction, uninfected cells were removed with puromycin selection. The library containing cells was treated with vehicle or ERX-41 for 2 weeks. Genomic DNA was extracted from same number of cells. The sgRNA cassette retrieved by PCR, followed by Next Generation Sequencing (NGS). NGS data was analyzed by MAGeCK (Li et al., Genome Biology, 2014 PMID: 25476604).

Lentiviral constructs cloning: LentiCRISPRv2 vector was used for CRSPR KO experiments. sgRNAs, which were chosen from human CRISPR knockout pooled library, were cloned into lentiCRISPRv2 vector³⁴. Human LIPA cDNA (GenBank: BC012287) or mouse LIPA cDNA (NM_021460) were clone into pWPI for over expression experiments. For TurboID experiment, the LIPA-TurboID sequence was cloned into pCW57-MCS1-2A-MCS2 vector. LentiCRISPR v2 was purchased from Addgene (Addgene #52961; //n2t.net/addgene:52961; RRID:Addgene_52961) (via Feng Zhang). pWPI was purchased from Addgene (Addgene #12254; //n2t.net/addgene:12254; RRID:Addgene_12254) (via Didier Trono). pCW57-MCS1-2A-MCS2 was purchased from Addgene (#71782) (via Adam Karpf). Human LIPA gene is referred as LIPA, while mouse LIPA gene is referred as mLIPA in this disclosure. Since LIPA overexpression was introduced in LIPA CRISPR KO background, to avoid being targeted by CRISPR-cas9, the LIPA sgRNA target sequence including PAM, tt aac cga att cct cat ggg agg, was mutated to tt aac cga att cct caC ggA agA in all of the LIPA overexpression constructs without protein coding change. In-Fusion Cloning kit (Takara) was used to generate different mutants of LIPA.

Live cell confocal and Airyscan imaging: To visualize ER structure, stable SUM-159 cell lines expressing mCherry-RAMP4 were established. pLenti-X1-hygro-mCherry-RAMP4 was purchased from Addgene (Addgene #118391; //n2t.net/addgene:118391; RRID:Addgene_118391) (via Jacob Corn).³⁵ The live cell images were acquired by Confocal Zeiss LSM880 Airyscan. ER tubule width was calculated by drawing 1px-wide line scans perpendicular to the long axis of individual ER tubules. In MATLAB, the peak intensity along each line scan was determined and the distance between the half maximum intensity on either side of the peak was measured. The code for this analysis is available at: //github.com/andmoo91/HalfMaxScript.

Lentivirus production: To generate lentivirus, lentiviral constructs (lentiCRISPR v2 for knocking out, pWPI for overexpression), along with the helper plasmids A8.9 and VsVg, were transfected into HEK293T cells using polyethylenimine (PEI, 1 mg/ml; Polysciences, Inc, Warrington, PA). Media was completely changed next day. Lentivirus was collected after additional 48-72 hours incubating. Filtered (0.45 μm) lentivirus containing media was used to infect cells using 6 μg/mL polybrene. Lentivirus containing media was completely changed next day.

Pull down assay: Cells were lysed in high salt lysis buffer (1.25 mM Hepes pH7.5, 400 mM NaCl, 0.5% NP-40, 5% glycerol and 2 mM MgCl₂) supplemented with 1 mM DTT and 1/100 Halt protease inhibitor cocktail (Thermo Scientific) on ice for 15 min, followed by 20 min of 20,000 g centrifuge at 4° C. The supernatant was mixed with 1.67× volume of no salt lysis buffer (1.25 mM Hepes pH7.5, 5% glycerol and 2 mM MgCl₂) supplemented with 1 mM DTT and 1/100 Halt protease inhibitor cocktail (Thermo Scientific). The lysate was incubated with biotinylated-ERX-11 or biotinylated-ERX-41 overnight, followed by one-hour incubation with M-270 streptavidin Dynabeads. After four times of washes with lysis buffer, samples were eluted by boiling in 2× Laemmli sample buffer. Bound proteins were resolved in SDS-PAGE followed by western blotting.

Western blotting: Whole cell lysates from breast cancer cells were prepared with 2× sample buffer (60 mM Tris-HCl, pH 6.8, 2% sodium dodecyl sulfate, 10% glycerol), and before loading mixed with loading buffer (5% 2-mercaptoethanol, 0.1% bromophenol blue) and boiled for denaturation, as previously described.³³ Proteins were separated by SDS-PAGE and subjected to Western analysis using antibodies. Antibodies used in this study are listed in Table S1. To detect nascent translated proteins, puromycin (final concentration 10 μg/mL) was added into cell plates 30 minutes before harvesting cells. Then samples were subjected to the Western blotting. Nascent proteins were detected by anti-puromycin antibody.

Real-time quantitative PCR (RT-qPCR): Total RNA was prepared from breast cancer cells using the RNeasy Mini Kit (Qiagen) according to the manufacturer protocols. Subsequently, total RNAs were reverse transcribed into cDNA using iScript Reverse Transcription Supermix (Bio-rad) and by using SYBR Green on an Illumina Real-Time PCR system. Primers employed are listed in Table S2.

Cellular Thermal Shift Assay (CETSA): Cellular Thermal Shift Assays were performed as described previously³⁶. Briefly, cells were treated with DMSO or 10 μM ERX-41 for 30 min, then trypsinized, pelleted and resuspended in PBS supplemented with Halt protease inhibitor cocktail (Thermo Scientific), with DMSO or 10 μM ERX-41. Resuspended cells were aliquoted into PCR strips. The cells were incubated in thermal cycler (Bio-rad) at gradient temperatures for 3 minutes, followed by incubation at 25° C. for 3 minutes. Then cells were snap-frozen in liquid nitrogen. Freeze-thaw the cells twice using liquid nitrogen. Then transfer samples into Eppendorf tubes, followed by briefly vortex and centrifuge at 20,000 g for 20 minutes at 4° C. Mix cleared cell lysates with ⅓ volume of 4× Laemmli sample buffer. After boiling, cell lysates were resolved in SDS-PAGE followed by western blotting.

LIPA staining in TNBC TMA and primary TNBC: A TNBC TMA was generated by the UT Southwestern Pathology Laboratory from 51 patients with high-grade. TN invasive breast carcinoma. All cases were treatment naive. This study was approved by the UT Southwestern University Institutional Review Board (no. STU032011-117). The diagnosis of TNBC was established by immunohistochemical analysis and confirmed by HER2 fluorescence in situ hybridization assay. The criteria for determining triple negativity were based on immunohistochemical staining and image quantitation of ER, PR, and HER2, and were confirmed by a board-certified breast cancer pathologist (Yan Peng) at UT Southwestern. Cerebellum tissue was included on TMA slide as negative control. Slides cut from the tissue blocks were immunostained for LIPA (SantaCruz, sc-58374, 1:500). An additional 20 slides were obtained from breast reduction surgeries from women without known breast cancer. Stained slides were scored manually per tissue core independently by a pathologist who was blinded to the clinical data. Immunostaining data were registered semiquantitatively in staining intensity (0, no staining; 1, weak staining; 2, moderate staining; and 3, intense staining, representative staining examples ranging from 0 to 3 are provided).

TNBC patient-derived explant (PDE) studies: For PDE studies excised tissue samples were processed and cultured ex vivo as previously described. De-identified patient tumors were obtained from the UTSW Tissue Repository after institutional review board approval (STU-032011-187). Inclusion criteria included women with prior histologic confirmation of TNBC who were undergoing surgical extirpation or biopsy of their primary tumor. Prior treatment with chemotherapy and/or radiation was allowed. Exclusion criteria included concurrent or prior diagnosis of other malignancies or prior evidence of ERα+ or HER2+ breast cancer. All cases were reviewed by the UTSW Tissue Repository in advance and patients were consented for their tissue to be used for laboratory research. Only de-identified information was shared with the laboratory. None of the laboratory personnel had access to additional patient information. No attrition was noted. Briefly, fresh tumor samples were incubated on gelatin sponges in culture medium containing 10% FBS, followed by treatment with either vehicle or presence of 2.5 μM ERX-41 for 72 h. Representative tissues were fixed in 10% formalin at 4° C. overnight and subsequently processed into paraffin blocks. Sections were then processed for immunohistochemical analysis.

Immunohistochemistry: Immunohistochemical studies were performed as described previously.⁷ For the immunohistochemical studies, tissue sections were blocked in background sniper (Biocare Medical, RS966L) followed by overnight incubation with primary antibodies anti-Ki-67 antibody (1:1000, GeneTex cat #GTX16667 RRID:AB_422351) (link), anti-CHOP antibody (1:2000, Sigma, Cat #HPA068416), anti-PERK (phospho T982) antibody (1:150, Abcam Cat #ab192591, RRID:AB_2728666) link)), anti-phospho-eIF2α (Ser51) (1:50, Cell signaling, cs3398) and subsequent secondary antibody incubation for 60 min at room temperature. Immunoreactivity was visualized by using the DAB substrate and counterstained with hematoxylin (Vector Lab, Inc.). Percent of Ki67 positive proliferating cells was calculated in five randomly selected high-power fields (×40).

Immunofluorescence: Cells were fixed with ice cold methanol, and blocked with 5% normal serum, 0.3% Triton X-100 PBS. After overnight incubation with primary antibodies at 4° C. and 4 times washes with 0.3% Triton X-100 PBS, samples were incubated with secondary antibodies at room temperature for one hour. After washing with 0.3% Triton X-100 PBS for 4 times, cells were further washed with Hank's balanced salt solution with calcium and magnesium for 4 times. Then samples were incubated with 0.2 μM ER-Tracker Red (Molecular Probes) at 37° C. for 30 min. Cells were further washed with Hank's balanced salt solution with calcium and magnesium for 4 times prior to mounting. The samples were imaged with confocal (Zeiss LSM 880).

Transmission Electron Microscopy: DMSO or ERX-41 treated cells were fixed with 2.5% glutaraldehyde (Electron Microscopy Sciences) in 0.1 M sodium cacodylate pH7.4 buffer (Electron Microscopy Sciences) for 20 min at room temperature. After rinsing with 0.1 M sodium cacodylate pH7.4 buffer, cells were further fixed with 1% Osmium with 0.8% K₃Fe(CN₆) in 0.1 M sodium cacodylate pH7.4 buffer. After pre-staining with 4% uranyl acetate in 50% ethanol, cells were dehydrated with series concentrations of ethanol (from 50% to 100%). After transitioning from propylene oxide to resin and embedding in Embed 812 resin (Electron Microscopy Sciences), cells were located using light microscopy and trimmed out. 60-70 nm-thin sections were cut, mounted on formvar-coated grids, and viewed by a transmission electron microscope (Tecnai G2 spirit; FEI) equipped with a LaB6 source using a voltage of 120 kV.

RNA-sequencing: mRNA-seq sequencing library construction was performed using TruSeq RNA Library Preparation Kit (Illumina) according to the manufacturer's instructions. FASTQ reads from Genomics Core at UTSW were mapped to Human GRCh38 genome using HiSAT2 (RRID:SCR_015530). Differential expression analysis is performed using DEseq2 (RRID:SCR_015687). Abundances of transcripts are calculated using ballgown (Frazee et al. 2014). The values of Fragments Per Kilobase of transcript per Million mapped reads (FPKM) were used for Gene Set Enrichment Analysis (GSEA). GSEA was performed by using the GSEA software (RRID:SCR_005724) and C5 ontology gene sets (14765 gene sets in total). mRNA-seq data from this study are available from NCBI GEO under accession #GSE168800.

Lipase activity assay: Lipase activity assays were performed as described previously (Dairaku, et al)³⁷. Briefly, a 0.345 mM substrate solution was prepared from 1.2 mL of 13.3 mM 4-MUP and 42 mL of 100 mM sodium acetate buffer pH 4.0, 1.0% (v/v) Triton X-100 and 3.0 mL of 0.5% (w/v) cardiolipin. For the enzyme reaction, 50 μL of the substrate in buffer solution, 40 μL of diluted cell lysate and 10 μL of DMSO or 30 μM Lalistat2 or 30 μM ERX-41 were combined in a black 96-well plate. The plates were sealed with an adhesive aluminum film and incubated in a 37° C. incubator for 1 h. The reactions were terminated using 200 μL of 150 mM EDTA at pH 11.5. The fluorescence of the plates were read immediately with synergy H1 fluorescence microplate reader (BioTek) using a 365-nm excitation filter and a 450-nm emission filter. The LIPA lipase activity was calculated by subtracting the enzymatic activity of the inhibited (with Lalistat2) reaction from that of the uninhibited (without Lalistat2) reaction.

Endo H and PNGase F assays: Endo H and PNGase F (New England Biolabs) assays were performed according to the manufacturer's manual. Briefly, cell lysates were denatured at 100° C. for 10 minutes after adding Glycoprotein Denaturing Buffer. For Endo H digestion, denatured cell lysate was incubated at 37° C. for 2 hours after adding GlycoBuffer 3 and Endo H. For PNGase F digestion, denatured cell lysate was incubated at 37° C. for 2 hours after adding GlycoBuffer 2, NP-40 (final concentration 1%) and PNGase F. The digested samples were mixed with ⅓ volume of 4× Laemmli sample buffer. After boiling, samples were resolved in SDS-PAGE followed by western blotting.

ELISpot. For ELISpot analysis of total IgM⁺ and IgG⁺ ASCs, Multi-Screen® filter plates (Millipore) were activated with 35% ethanol, washed with PBS, and coated with anti-IgM (SouthernBiotech, Cat. #1020-01) or anti-IgG (SouthernBiotech, Cat. #1030-01) in PBS. Single bone marrow cell suspensions were prepared, as above, and cultured at 250,000 cells/ml in RPMI 1640 medium (Invitrogen) supplemented with FBS (10% v/v, Invitrogen), penicillin-streptomycin/amphotericin B (1% v/v) and 50 μM β-mercaptoethanol (RPMI-FBS) at 37° C. for 16 h. After supernatants were removed, plates were incubated with biotinylated goat anti-mouse IgM (SouthernBiotech, Cat. #1020-08) or goat anti-mouse IgG1 (SouthernBiotech, Cat. #1070-08) Ab for 2 h and, after washing, incubated with HRP-conjugated streptavidin. Plates were developed using the Vectastain AEC peroxidase substrate kit (Vector Laboratories). The stained area in each well was quantified using the CTL ImmunoSpot software (Cellular Technology) and depicted as the number of spots for quantification

Flow cytometry: Single cell suspensions were flushed from tibia and fibula using sterile DPBS using a 10 ml syringe and 30 G needle. Red blood cells were removed by incubation with ACK lysis buffer (Lonzo) for 2 m. Bone marrow cells (2×10⁶) were first stained in Hank's Buffered Salt Solution plus 0.1% BSA (HBSS-BSA) for 20 m with fixable viability dye (FVD, eFluor™ 506, eBioscience™, Cat. #50-246-097) and fluorophore-labeled mAbs to surface markers, including CD3 (APC-Cy7, Clone 17A2, BioLegend, Cat. #100221), IgD (FITC, Clone 11-26c.2a, BioLegend, Cat. #405704), CD138 (PE-Cy7, Clone 281-2, BioLegend, Cat. #142514), SCA-1 (PerCP, Clone D7, BioLegend, Cat. #108121), B220 (BV421, Clone RA3-6B2, BioLegend, Cat. #103240), in the presence of mAb Clone 2.4G2, which blocks FcγII and FcγIII receptors. After washing, cells were then fixed and permeablized by incubation for 1 h in 250 μl BD Cytofix/Cytoperm™ buffer (BD Fixation/Permeablization Kit, Cat. #554714) at 4° C. After washing twice with the BD Perm/Wash™ buffer, cells were counted again and 10⁶ cells were resuspended in 100 μl of BD Cytofix/Cytoperm™ buffer for intracellular staining with anti-Igκ mAb (PE, e-Bioscience™, Clone 1871, Cat. #MKAPPA04) for 30 m. After washing with BD Perm/Wash™ buffer, cells were analyzed by LSRII (BD). FACS data were analyzed by the FlowJo® software (BD).

TurboID pull down: TurboID pull down assay was performed as described previously (Cho et al., PMID: 30125270 & 33139955). Briefly, pCW57-LIPA-TurboID transduced cells were treated with or without 1 mg/mL doxycycline for 48 hour, then were treated with 50 mM biotin for 15 minutes and then harvested and lysed in RIPA buffer (Thermo Scientific) supplemented with Halt protease inhibitor cocktail (Thermo Scientific) and Phosphatase Inhibitor Cocktail set I and set II (MilliporeSigma) on ice for 15 min, followed by 20 min of 20,000 g centrifuge at 4° C. Cleared cell lysates were incubated with M-270 streptavidin Dynabeads overnight. Then the beads were washed in a series buffers: twice with RIPA buffer (2 min), once with 1 m KCl (2 min), once with 0.1 M Na₂CO₃ (10 s), once with 2 M urea in 10 mM Tris-HCl (pH 8.0) (10 s), and twice with RIPA buffer (2 min). Then elute enriched material from the beads by boiling samples in 4× Laemmli buffer supplemented with 2 mM biotin and 20 mM DTT at 95° C. for 10 min. Eluted samples were resolved in SDS-PAGE and followed by mass spectrometry by Lumos.

Gene Ontology: Gene ontology analysis were performed using the Database for Annotation, Visualization and Integrated Discovery (DAVID) bioinformatics resource v6.8 (david.ncifcrf.gov/tools.jsp) (Nature Protocols 2009; 4(1):44 & Nucleic Acids Res. 2009; 37(1):1, PMID: 19131956 & 19033363).

Venn Diagram Analysis: The Ven diagram analysis was done using BioVenn. (world-wide-webe at biovenn.nl/index.php) (BioVenn—a web application for the comparison and visualization of biological lists using area-proportional Venn diagrams T. Hulsen, J. de Vlieg and W. Alkema, BMC Genomics 2008, 9 (1): 488, PMID: 18925949).

TABLE S1
Antibodies

| Target | Supplier | Cat. # | Clone | Lot # | Application |
|---|---|---|---|---|---|
| ARID1A | Santa Cruz | sc-32761 | PSG3 | D2319 | WB |
| ATF-4 | Santa Cruz | sc-390063 | B-3 | C2921 | WB |
| Calreticulin | Cell | 12238 | D3E6 | 5 | WB |
| | Signaling | | | | |
| CHOP | Sigma | HPA068416 | Polyclonal | R100284 | WB, IHC |
| DNAJC10 | ABclonal | A12260 | Polyclonal | 0053040201 | WB |
| eIF2a | Santa Cruz | sc133132 | D-2 | G2420 | WB |
| ERalpha | Santa Cruz | sc-8002 | F-10 | E2814 | IHC |
| Histone H3 | Abcam | ab8898 | Polyclonal | GR148830-2 | WB |
| IRE1a | Cell | 3294 | 14C10 | 100000 | WB |
| | Signaling | | | | |
| Ki67 | GeneTex | GTX16667 | SP6 | 822002798 | IHC |
| LAL | Santa Cruz | sc-58374 | 9G7F12 | L1813 | IHC, WB |
| LAL | Invitrogen | PA5-84326 | Polyclonal | VD2980319, | WB |
| | | | | VJ3111553B | |
| LAMP2 | Santa Cruz | SC-18822 | H4B4 | G2517 | WB |
| LIMP2 | Novus | NB400-129SS | Polyclonal | B-8 | IF |
| MANF | Bethyl | A305-572A-T | Polyclonal | 1 | WB |
| Myc | Cell | 2276 | 9B11 | 24 | IF |
| | Signaling | | | | |
| P3H2 | ABclonal | A8068 | Polyclonal | 0033870201 | WB |
| PDIA3 | ABclonal | A1085 | Polyclonal | 0056780201 | WB |
| PDIA5 | ABclonal | A14476 | Polyclonal | 0113680101 | WB |
| p-eIF2a | Cell | 9721 | Polyclonal | 21 | IHC, WB |
| | Signaling | | | | |
| PERK | Cell | 5683 | D11A8 | 5, 6 | WB |
| | Signaling | | | | |
| p-IRE1a | Abcam | ab124945 | EPR5253 | GR271918-22 | WB |
| POGLUT2 | Santa Cruz | sc-390065 | K0921 | C-3 | WB |
| p-PERK | Abcam | ab192591 | Polyclonal | GR18597-59 | IHC, WB |
| SLC5A3 | Proteintech | 21628-1-AP | Polyclonal | 14874 | WB |
| SOAT1 | Santa Cruz | sc-69836 | ACAT-1 | B2621 | WB |
| TMEM208 | Proteintech | 23882-1-AP | Polyclonal | 6 | WB |
| Vinculin | Cell | 13901 | E1E9V | 822002798 | WB |
| | Signaling | | | | |

TABLE S2
Primers

| Name | Sequence | SEQ ID NO: | Application |
|---|---|---|---|
| SXBP1_F | CTGAGTCCGAATCAGGTGCAG | 1 | qPCR |
| XBP1_R | ATCCATGGGGAGATGTTCTGG | 2 | qPCR |
| GAPDH_F | GGATTTGGTCGTATTGGG | 3 | qPCR |
| GAPDH_R | GGAAGATGGTGATGGGATT | 4 | qPCR |
| HSPA5_F | TGTTCAACCAATTATCAGCAAACTC | 5 | qPCR |
| HSPA5_R | TTCTGCTGTATCCTCTTCACCAGT | 6 | qPCR |
| DDIT3_F | AGAACCAGGAAACGGAAACAGA | 7 | qPCR |
| DDIT3_R | TCTCCTTCATGCGCTGCTTT | 8 | qPCR |
| HNRNPC_F | CACCGTATCAGGAAACACTTCACGA | 9 | sgRNA |
| HNRNPC_R | AAACTCGTGAAGTGTTTCCTGATAC | 10 | sgRNA |
| STOML2_F | CACCGTCAGGCAAGCTACGGTGTGG | 11 | sgRNA |
| STOML2 R | AAACCCACACCGTAGCTTGCCTGAC | 12 | sgRNA |
| ASNA1_1F | CACCGTTCCCCACCATCGTGGAGCG | 13 | sgRNA |
| ASNA1_1R | AAACCGCTCCACGATGGTGGGGAAC | 14 | sgRNA |
| ASNA1_2F | CACCGCTGAAGTGGATCTTCGTCGG | 15 | sgRNA |
| ASNA1_2R | AAACCCGACGAAGATCCACTTCAGC | 16 | sgRNA |
| FUS_F | CACCGGCCACCACCACTACTCATGG | 17 | sgRNA |
| FUS_R | AAACCCATGAGTAGTGGTGGTGGCC | 18 | sgRNA |
| ACACA_F1 | CACCGAATGCATGCGGTCTATCCGT | 19 | sgRNA |
| ACACA_R1 | AAACACGGATAGACCGCATGCATTC | 20 | sgRNA |
| ACACA_F2 | CACCGTGATGGGTCCATGATAACCG | 21 | sgRNA |
| ACACA R2 | AAACCGGTTATCATGGACCCATCAC | 22 | sgRNA |
| LIPA_F1 | CACCGTTAACCGAATTCCTCATGGG | 23 | sgRNA |
| LIPA_R1 | AAACCCCATGAGGAATTCGGTTAAC | 24 | sgRNA |
| LIPA_F2 | CACCGCAAGAACAAGTGTATTATGT | 25 | sgRNA |
| LIPA_R2 | AAACACATAATACACTTGTTCTTGC | 26 | sgRNA |
| TMEM208_F1 | CACCGAAAAGAAGACCAACGTCACA | 27 | sgRNA |
| TMEM208_R1 | AAACTGTGACGTTGGTCTTCTTTTC | 28 | sgRNA |
| TMEM208_F2 | CACCGGTGCCATCGAGCTCATAGAG | 29 | sgRNA |
| TMEM208_R2 | AAACCTCTATGAGCTCGATGGCACC | 30 | sgRNA |
| SLC5A3_F1 | CACCGCCAGCTTCATCACCAGGCGT | 31 | sgRNA |
| SLC5A3_R1 | AAACACGCCTGGTGATGAAGCTGGC | 32 | sgRNA |
| SLC5A3_F2 | CACCGAGCATGGGTGCCAATCATCG | 33 | sgRNA |
| SLC5A3_R2 | AAACCGATGATTGGCACCCATGCTC | 34 | sgRNA |
| ARID1A_F1 | CACCGCAGCAGAACTCTCACGACCA | 35 | sgRNA |
| ARID1A_R1 | AAACTGGTCGTGAGAGTTCTGCTGC | 36 | sgRNA |
| ARID1A_F2 | CACCGCACATCCCTGACCCAACCTG | 37 | sgRNA |
| ARID1A_R2 | AAACCAGGTTGGGTCAGGGATGTGC | 38 | sgRNA |
| SOAT1_F1 | CACCGTTAGCTGAATTTAGTACCCG | 39 | sgRNA |
| SOAT1_R1 | AAACCGGGTACTAAATTCAGCTAAC | 40 | sgRNA |
| SOAT1_F2 | CACCGTCCCTAGAGACACCTAGTAA | 41 | sgRNA |
| SOAT1 R2 | AAACTTACTAGGTGTCTCTAGGGAC | 42 | sgRNA |
| SMAP2_F1 | CACCGGACCAGCAATACCCTAGAGA | 43 | sgRNA |
| SMAP2_R1 | AAACTCTCTAGGGTATTGCTGGTCC | 44 | sgRNA |
| sgLIPA- | CCTCACGGAAGAAAGAACCATTCTGACA | 45 | mutation |
| 1_mut_F | AAGGTCCC | | |
| sgLIPA- | CTTTCTTCCGTGAGGAATTCGGTTAAGG | 46 | mutation |
| 1_mut_R | CACAG | | |
| sgLIPA- | GAGCAGGTCTACTACGTGGGTCATTCTC | 47 | mutation |
| 2_mut_F | AAGGCACC | | |
| sgLIPA- | GTAGTAGACCTGCTCTTGGCCAGTTTTA | 48 | mutation |
| 2_mut_R | TTCAGAATG | | |
| L239P_F | TGGAAATCCCTGTTTTCTTCTGTGTGGAT | 49 | mutation |
| | TTAAT | | |
| L239P_R | AAACAGGGATTTCCACAGAGCTCCTTCA | 50 | mutation |
| L242P_F | CTGTTTTCCTCTGTGTGGATTTAATGAGA | 51 | mutation |
| | GAAAT | | |
| L242P_R | CACAGAGGAAAACAGAGATTTCCACAG | 52 | mutation |
| | AGCT | | |
| L242P/L243P_F | GTTTTCCTCCGTGTGGATTTAATGAGAG | 53 | mutation |
| | AAATTT | | |
| L242P/L243P_R | CACACGGAGGAAAACAGAGATTTCCAC | 54 | mutation |
| | AGAGCT | | |
| L239P/L242P/ | TCCCTGTTTTCCTCCGTGTGGATTTAATG | 55 | mutation |
| L243P_F | AGAGAAATTT | | |
| L239P/L242P/ | GGAGGAAAACAGGGATTTCCACAGAGC | 56 | mutation |
| L243P_R | TCCTTCAGT | | |
| L239V/C240F_F | GAAATGTCTTCTTTCTTCTGTGTGGATTT | 57 | mutation |
| | AATGAGA | | |
| L239V/C240F_R | GAAAGAAGACATTTCCACAGAGCTCCTT | 58 | mutation |
| | CAGT | | |
| ΔLXXLL_F | TCTGTGGAGGATTTAATGAGAGAAATTT | 59 | mutation |
| | AAAT | | |
| ΔLXXLL_R | TAAATCCTCCACAGAGCTCCTTCAGT | 60 | mutation |
| H274Y_F | ACATGTTATACTGGAGCCAGGCTGTTA | 61 | mutation |
| H274Y_R | TCCAGTATAACATGTTTTGCACAGAAGT | 62 | mutation |
| | TCCA | | |

Animal Studies: All animal experiments were performed using UTHSA and UTSW IACUC approved protocols. For cell based xenograft tumor assays, TNBC cells (2×10⁵-2×10⁶) were mixed with an equal volume of matrigel and implanted in the mammary fat pads of 6-week-old female athymic nude mice as described³³. BALB/c mice were used for D2A1 syngeneic model. Once tumors reached measurable size, mice were divided into control (vehicle) and ERX-41 (10 mg/kg/oral) treatment groups. Mice bearing TNBC PDX tumors were purchased from Jackson laboratory (TM00089; TM00096; TM00098). TNBC PDX line UTPDX0001 establishment was earlier described³⁸. WHIM-20 ER+PDX model was purchased from Horizon Labs. OCa-PDX-38 was received from UTHSA ObGyn tissue procurement core. When tumors reached ˜750 mm³ they were dissected into 2 mm³ pieces and implanted into the mammary fat pads of 6 weeks old female SCID or NSG mice. When the tumor volume reached ˜200 mm³, mice were randomized for treatment. The mice were monitored daily for adverse toxic effects and tumor volume was measured every 3-4 days using calipers. For establishing ES2 tumors, ES2/GFP/LUC cells were injected i.p. and tumors were measured twice a week using Xenogen in vivo imaging system. At the end of each experiment, the mice were euthanized, and the tumors were removed, weighed and processed for histological studies and protein analysis. Dose were selected based on pilot MTD study of 10, 50 100 mg/kg of ERX-41 for 14 days using C57BL6 mice. The mice were monitored daily for adverse toxic effects. IHC analysis was conducted as described previously³⁹ and immunoreactivity was visualized using DAB substrate and counterstained with hematoxylin (Vector Laboratories Inc. Burlingame, CA). Control rabbit IgG staining was used as a negative control. The sections were scored by two independent evaluators blinded to the patient's clinical status.

Knockout studies: For xenograft knockout tumor models, 1.8×10⁶SUM159 parental, or 1.8×10⁶SUM159-LIPA KO cells were harvested with 1% trypsin-EDTA, washed and resuspended in sterile PBS. Cells were initially implanted into the mammary fat pads of six-week-old female C.B-17 (SCID) mice purchased from Envigo (Indianapolis, IN, USA). Mice were monitored twice weekly for tumor growth measured by Vernier calipers where tumor volume was calculated by the formula (length×width²)/2. Tumors were dissected into 2 mm cubes when they reached 800 mm³ in size and subsequently implanted into the mammary fat pad of twelve SCID female mice. Once tumor fragments reached 150 mm³ in size, mice were randomly divided into vehicle group (n=6, control, 0.3% Hydroxy cellulose, i.p.) and treatment group (n=6, ERX-41, 10 mg/kg/day/i.p.). Institutional guidelines were followed to determine experimental end points.

Analytical LC-MS/MS conditions: Compound levels in plasma and tissues for in vivo pharmacokinetic (PK) studies were monitored by LC-MS/MS using an AB Sciex (Framingham, MA) 6500⁺ QTRAP® mass spectrometer coupled to a Shimadzu (Columbia, MD) Prominence LC. Analytes were detected with the mass spectrometer in positive MRM (multiple reaction monitoring) mode by following the precursor to fragment ion transition 705.3→592.3. An Agilent C18 XDB column (5 micron, 50×4.6 mm) was used for chromatography for PK studies with the following conditions: Buffer A: dH₂O+0.1% formic acid, Buffer B: methanol+0.1% formic acid, 0-1.0 min 5% B, 1.0-2 min gradient to 98% B, 2-3.0 min 98% B, 3.0-3.2 min gradient to 5% B, 3.2-4.5 min 5% B. Tolbutamide (transition 271.2 to 91.2) from Sigma (St. Louis, MO) was used as an internal standard (IS). Pharmacokinetic studies were performed in MDA-MB-231 xenograft tumor-bearing female NOD-SCID mice. ERX-41 was given either orally or intraperitoneally (10 mg/kg single dose). Animals were sacrificed in groups of three, blood was obtained by cardiac puncture at each time point (0, 0.5, 1.5, 3, 6 and 24 hours post dose) using the anticoagulant EDTA and plasma isolated by centrifugation. Tumor and liver were also collected and snap frozen in liquid nitrogen after rinsing with PBS to remove surface adhering blood. Tissues were homogenized in a 3-fold volume (weight by volume) of PBS to generate a homogenate. 100 μl of plasma or tissue homogenate was mixed with 200 μl of methanol containing 0.15% formic acid and 15 ng/mL tolbutamide IS. The samples were vortexed 15 sec, incubated at room temp for 10 min and spun twice at 16,100×g 4° C. in a refrigerated microcentrifuge. The amount of compound present in plasma, tumor and liver was quantified by LC-MS/MS to determine the amount of compound in each matrix. Standard curves were generated using blank plasma (Bioreclamation, Westbury, NY, RRID:SCR_004728) or blank tissue homogenate spiked with known concentrations of test compound and processed as described above. The concentrations of drug in each time-point sample were quantified using Analyst 1.7.1 software (Sciex). A value of 3-fold above the signal obtained from blank plasma or tissue homogenate was designated the limit of detection (LOD). The lower limit of quantitation (LLOQ) was defined as the lowest concentration at which back calculation yielded a concentration within 20% of theoretical and which was above the LOD. The LLOQ for plasma and tumor was 0.1 ng/mL and 1 ng/mL in liver. PK parameters, Cₘₐₓ, Tₘₐₓ, Terminal T½ (calculated as Ln(2)/λ_{z}), AUCₗₐₛₜ (area under the concentration time curve to the last measured value determined by linear trapezoidal rule), Cl (clearance measured as Dose/AUC_{inf}), and V_{z} (volume of distribution based on the terminal phase=Dose/AUC_{inf}*λ_{z}) were calculated using the noncompartmental analysis tool of Phoenix WinNonLin version 8.01 (Certara/Pharsight, Sunnyvale, CA, RRID:SCR_003163).

Subcellular fractionation: SUM-159-KO+WT cell pellets were suspended in PBS+0.5% BSA and subjected to sonication and sequential centrifugation (2000 g for 12 min, 15000 g for 60 min). Pellet after 2000 g centrifugation is nuclear fraction. Supernatant after 15000 g centrifugation is ER fraction. Pellet after 15000 g centrifugation is lysosome fraction. Enrichment for nuclear, ER and lysosomal fractions were validated by immunoblotting for histone H3, calreticulin and LAMP2 respectively.

Mass spectrometry based DIA analyses of whole cell lysates: Sum159 cells were treated with vehicle or ERX-41 (500 nM). After 6 h of treatment, cells were pelleted and snap-frozen and then lysed in buffer containing 5% SDS/50 mM triethylammonium bicarbonate (TEAB) in the presence of protease and phosphatase inhibitors (Halt; Thermo Scientific) and nuclease (Pierce™ Universal Nuclease for Cell Lysis; Thermo Scientific). Aliquots corresponding to 100 μg protein (EZQ™ Protein Quantitation Kit; Thermo Scientific) were reduced with tris (2-carboxyethyl)phosphine hydrochloride (TCEP), alkylated in the dark with iodoacetamide and applied to S-Traps (mini; Protifi) for tryptic digestion (sequencing grade; Promega) in 50 mM triethylammonium bicarbonate (TEAB). Peptides were eluted from the S-Traps with 0.2% formic acid in 50% aqueous acetonitrile and quantified using Pierce™ Quantitative Fluorometric Peptide Assay (Thermo Scientific). Data-independent acquisition mass spectrometry (DIA-MS) was conducted on an Orbitrap Fusion Lumos mass spectrometer (Thermo Scientific). On-line HPLC separation was accomplished with an RSLC NANO HPLC system (Thermo Scientific/Dionex: column, PicoFrit™ (New Objective; 75 μm i.d.) packed to 15 cm with C18 adsorbent (Vydac; 218MS 5 μm, 300 Å); mobile phase A, 0.5% acetic acid (HAc)/0.005% trifluoroacetic acid (TFA) in water, mobile phase B, 90% acetonitrile/0.5% HAc/0.005% TFA/9.5% water; gradient 3 to 42% B in 120 min; flow rate, 0.4 μl/min. A pool was made of the 6 samples (three replicates from each group), and 2-μg peptide aliquots were analyzed using gas-phase fractionation and 4-m/z windows (30k resolution for precursor and product ion scans, all in the orbitrap) to create a DIA chromatogram library⁴⁰ by searching against a Prosit-generated predicted spectral library⁴¹ based on the UniProt_human_20191022 protein sequence database. Experimental samples were blocked by replicate and randomized within each replicate for sample preparation and analysis; injections of 2 μg of peptides and a 2-h HPLC gradient were employed. MS data for experimental samples were acquired in the orbitrap using 8-m/z windows (staggered; 30k resolution for precursor and product ion scans) and searched against the chromatogram library. Scaffold DIA (v3.1.0; Proteome Software) was used for all DIA-MS data processing. Gene ontology analysis of differentially expressed proteins was conducted using the biological processes and cellular component, focusing on the group that exhibited ≥1.5-fold change comparing vehicle and ERX-41.

Statistical Analyses. Statistical differences between groups were analyzed with either a t-test or ANOVA as appropriate using GraphPad Prism 8 software (RRID:SCR_002798). All data represented in plots represent the mean t SEM. A value of p<0.05 was considered as statistically significant. For animal studies, sample size of tumors/treatment was derived using effect information from previous studies and calculations were based on a model of unpaired data power=0.8; p<0.05. All in vitro assays were performed in biological replicates in technical triplicate. For animal studies, sample size of tumors/treatment was derived using effect information from previous studies and studies and calculations were based on a model of unpaired data power=0.8; p<0.05.

### Example 2—Synthetic Methods

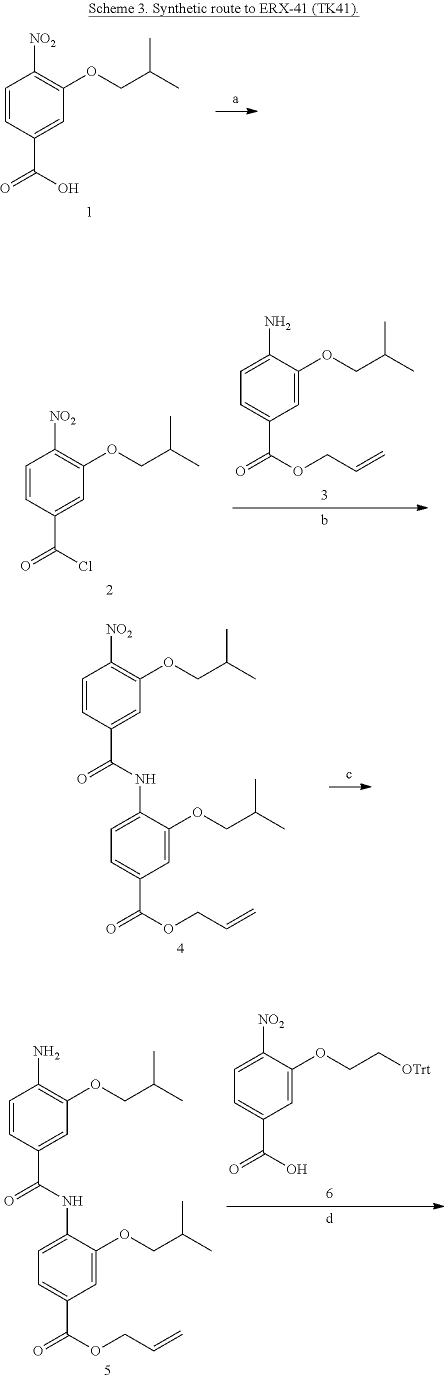

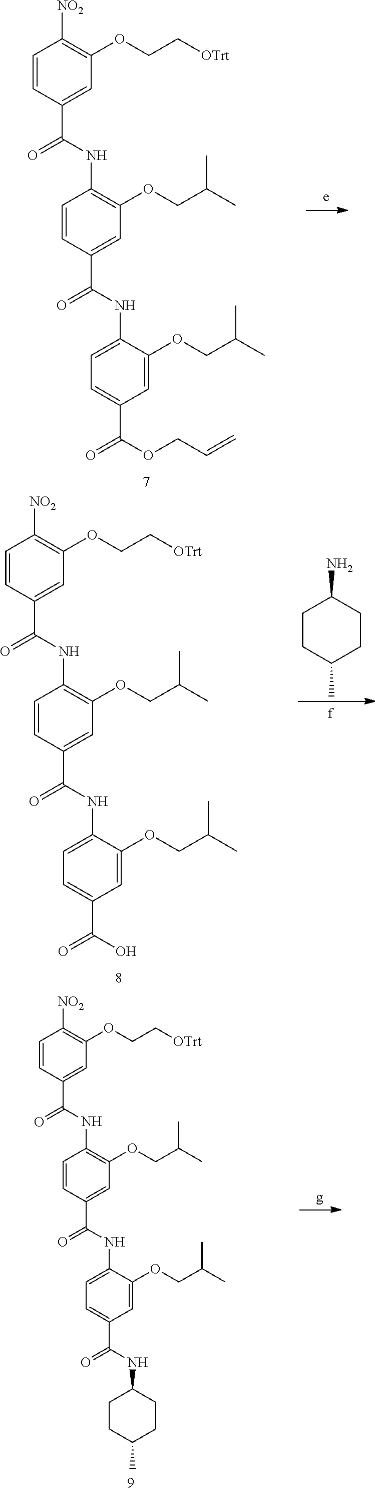

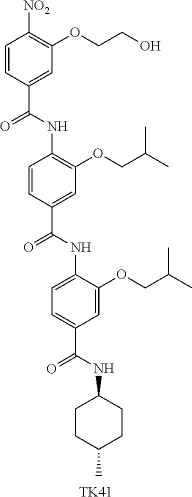

Reagents and conditions: (a) (COCl)₂, cat. DMF, DCM, rt, 2 h; (b) DIEA, DCM, rt, 24 h; (c) SnCl₂, DMF, rt, 12 h; (d) HATU, DIEA, DMF, rt, 24 h; (e) Pd(PPh₃)₄, PhSiH₃, THF, rt, 1 h; (f) HATU, DIEA, DMF, rt, 24 h; (g) conc. HCl, rt, 24 h.

Compound 4: A 250 mL round-bottomed flask was charged with compound 1 (5.45 g, 22.8 mnmol), DCM (100 mL), oxalyl chloride (2.6 mL, 30.1 mmol) and 2 drops of DMF. The reaction mixture was stirred at room temperature for 2 h and then concentrated under reduced pressure. The resulting compound 2 was dissolved in DCM (20 mL) and slowly added to a solution of compound 3 (3.8 g, 15.2 mmol), DIEA (5.3 mL, 30.4 mmol) and DCM (100 mL). The reaction mixture was stirred at room temperature for 24 h, and then was washed with 1 N HCl (50 mL), saturated NaHCO₃ (50 mL) and brine (50 mL). The organic layer was dried over Na₂SO₄, filtered, and concentrated under reduced pressure to yield the crude product. Purification by crystallization from EtOAc/hexanes (1:4) gave compound 4 as a light yellow solid (5.1 g, 71%).

Compound 5: A 250 mL round-bottomed flask was charged with compound 4 (4.7 g, 10.0 mmol), DMF (100 mL), and SnCl₂·2H₂O (6.8 g, 30.0 mmol). The reaction mixture was stirred at room temperature for 12 h and then diluted with EtOAc (200 mL) and 1 N HCl (200 mL). The organic layer was separated and washed with 1 N HCl (100 mL) and brine (100 mL). The organic layer was dried over Na₂SO₄, filtered, and concentrated under reduced pressure to yield the crude product. Purification by flash chromatography (hexanes/EtOAc 4:1) gave the compound 5 as a light yellow solid (3.6 g, 82%).

Compound 7: A 250 mL round-bottomed flask was charged with compound 5 (3.6 g, 8.2 mmol), compound 6 (6.2 g, 13.2 mmol), HATU (6.7 g, 17.6 mmol), DMF (100 mL), and DIEA (4.6 mL, 26.4 mmol). The reaction mixture was stirred at room temperature for 24 h and then diluted with EtOAc (300 mL) and 0.5 N HCl (200 mL). The organic layer was separated and washed with 0.5 N HCl (100 mL) and brine (100 mL). The organic layer was concentrated under reduced pressure to yield the crude product. Purification by crystallization from EtOAc gave compound 7 as a light yellow solid (5.6 g, 77%).

Compound 8: A 250 mL round-bottomed flask was charged with compound 7 (5.3 g, 5.9 mmol) and THF (100 mL). Then, Pd(PPh₃)₄ (0.69 g, 0.60 mmol) and PhSiH₃ (1.5 mL, 12.2 mmol) were added to the reaction mixture. The reaction mixture was stirred at room temperature for 1 h. The resulting solid was filtered, washed with ether and dried in vacuo to give compound 8 as a white sold (4.9 g, 97%).

Compound 9: A 100 mL round-bottomed flask was charged with compound 8 (2.7 g, 3.2 mmol), HATU (1.4 g, 3.7 mmol), DMF (30 mL), trans-4-methylcyclohexylamine (0.73 g, 6.4 mmol), and DIEA (1.2 mL, 6.9 mmol). The reaction mixture was stirred at room temperature for 24 h and then diluted with EtOAc (100 mL) and 0.5 N HCl (50 mL). The organic layer was separated and washed with 0.5 N HCl (50 mL) and brine (50 mL). The resulting solid was filtered, washed with EtOAc and dried in vacuo to give compound 9 as a white sold (1.75 g). The product was used in the next reaction without further purification.

ERX-41 (TK41): A 500 mL round-bottomed flask was charged with compound 9 (1.75 g), THF (300 mL) and conc. HCl (30 mL). The reaction mixture was stirred at room temperature for 24 h and then concentrated under reduced pressure. The resulting solid was filtered, washed with MeOH and dried in vacuo to give TK41 as a light yellow solid (1.3 g, 57% over 2 reaction steps).

### Example 3—Results

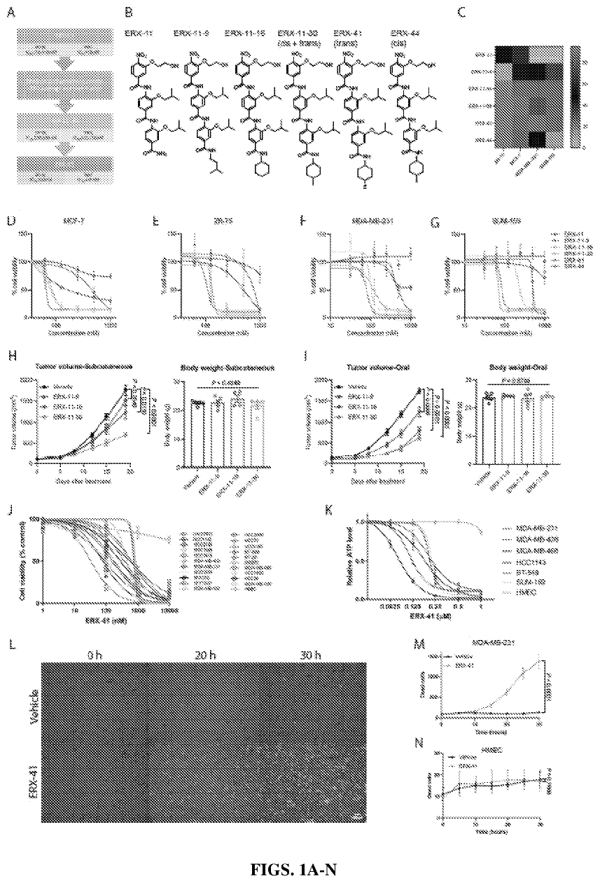

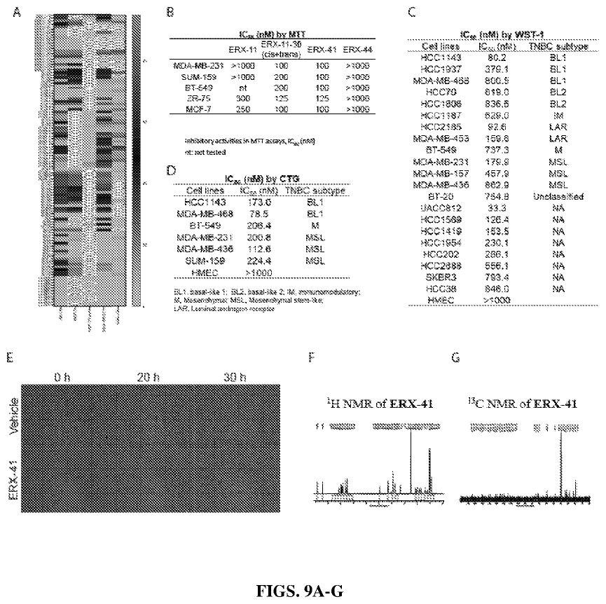

Identification of ERX-41 as a therapeutic hit against TNBC. Previously, the inventors noted that ERX-11 had anti-proliferative activity (IC₅₀ of 200-500 nM) against ERα-positive breast cancer cells, as shown for MCF-7 and ZR-75 (FIG. 1A, structure in FIG. 1B, FIG. 1C heatmap, FIGS. 1 D-E). Importantly, ERX-11 had no significant activity (IC₅₀>10 μM) against TNBC cells, as shown for SUM-159 and MDA-MB-231 (FIG. 1A, FIG. 1C heatmap, FIGS. 1F-G). During lead optimization of D2 and ERX-11, the inventors designed and synthesized >200 oligobenzamide analogs (FIG. 1A). These analogs were designed by changing O-alkoxy substituents of D2 and ERX-11 with a variety of functional groups with different structures and physicochemical properties, including structurally diverse alkyl chains at the C-terminal carboxamide position. While the primary goal was to improve the potency for ERα-positive breast cancer cells, the inventors were surprised to find that some oligobenzamides (ERX-11-9, ERX-11-16 and ERX-11-30) (structures shown in FIG. 1B, heatmap in FIG. 1C and FIG. 9A, proliferation data in FIGS. 1D-G, IC₅₀ data in FIGS. 9B-D) had potent activity against both ERα-positive breast cancer and TNBC cells. Interestingly, these three oligobenzamides were all derivatives of the C-terminal carboxamide of ERX-11. To evaluate if these analogs could have therapeutic utility, the inventors then tested each of these agents against established xenografts in nude mice (FIGS. 1H-I). Since ERX-11 was orally bioavailable, the inventors tested both oral and subcutaneous administration of these oligobenzamides to avoid any potential issues with altered biodistribution. Importantly, none of these oligobenzamides showed toxicity in mice upon either subcutaneous or oral administration, as evidenced by a lack of significant changes in mice body weights (FIGS. 1H-I), after 3 weeks of treatment. While all three oligobenzamides (ERX-11-9, ERX-11-16 and ERX-11-30) decreased tumor growth rates (FIGS. 1H-I), ERX-11-30 was the most potent with both oral and subcutaneous administration in vivo (FIGS. 1H-I).

Since ERX-11-30 is a racemic mixture of both cis- and trans-4-methylcyclohexylamide of ERX-11, the inventors synthesized two stereochemically defined isoforms: ERX-11-41 with trans configuration and ERX-11-44 with cis configuration (shortened hereafter as ERX-41 and 44 respectively for simplicity) to determine stereochemical impact of the 4-methyl group in the cyclohexane structure (structures shown in FIG. 1B) on the proliferation of breast cancer cells. Interestingly, only the trans-isoform ERX-41 showed antiproliferative activity against both TNBC and ERα-positive breast cancer cells with an IC₅₀ of 100-125 nM in MTT cell viability assays (FIG. 1C heatmap, FIGS. 1D-1G). As expected, ERX-41 (IC₅₀ of 100-125 nM) is more potent than ERX-11-30 (IC₅₀ of 100-200 nM) (FIGS. 1C-1G, FIG. 9B). In contrast, the cis-isoform of ERX-11-30, ERX-44 did not have significant antiproliferative activity against these cells (FIGS. 1C-1G, FIG. 9B). While ERX-41 (NMR characteristics shown in FIGS. 9F-G) was incrementally more potent than ERX-11 (IC₅₀ 100-125 nM vs IC₅₀ 200-500 nM) in ERα-positive breast cancer, the potency in TNBC was dramatically (>100-fold) enhanced from IC₅₀>10 μM to 100 nM (FIG. 9B). Based on these findings, the inventors designated ERX-41 as a viable hit in TNBC from the screen of a curated oligobenzamide library of D2 and ERX-11 analogs.

ERX-41 has potent activity against TNBC cells in vitro. To evaluate the generalizability of their findings, the inventors then evaluated ERX-41 in a large number of TNBC and ERα-positive cell lines. In 21 human preclinical TNBC cell line models, representing all six molecular subtypes of TNBC, ERX-41 had potent anti-proliferative activity with an IC₅₀<500 nM using WST-1 assays and <250 nM using CellTiter-Glo cell viability assays (FIGS. 1J-K, tabulated in FIGS. 9C-D). ERX-41 did not have significant effect against normal human mammary epithelial cells (HMEC) at >1 μM with either the WST, MTT or CellTiter-Glo assays (FIGS. 1J-K, tabulated in FIGS. 9C-D). In contrast, in ERα-positive cell lines, ERX-41 has variable anti-proliferative activity with IC₅₀ ranging from 50-1000 nM using WST assays (tabulated in FIGS. 9C-D) and 100-125 nM using CellTiter-Glo assays (FIGS. 1D-E). Live cell imaging studies indicated that ERX-41 significantly induced cell death in TNBC cells within 30 hours of treatment in a vast majority of the cells (>90%) compared to control (<1%) (still images in FIG. 1L, quantitation in FIG. 1M). By contrast, ERX-41 did not significantly induce cell death in HMEC cells (quantitation in FIG. 1N, still images in FIG. 9E).

ERX-41 has potent activity against TNBC in vivo. Since ERX-41 had potent activity against both TNBC and ERα-positive breast cancer, the inventors decided to first evaluate if ERX-41 could be safely tolerated in vivo. Daily oral or intraperitoneal administration of ERX-41 up to 100 mg/kg doses were well tolerated by mice, with no clear evidence of toxicity. Pharmacokinetic studies indicated that ERX-41 was orally bioavailable, with peak detectable levels in the plasma at 4 h after oral administration (10 mg/kg single dose). Additionally, ERX-41 was detectable within 1.5 h in established subcutaneous MDA-MB-231 xenograft tumors after oral or intraperitoneal administration (FIG. 2A).

The inventors then demonstrated that oral administration of ERX-41 (10 mg/kg/day) significantly decreased the growth of established MDA-MB-231 TNBC xenografts in vivo (FIG. 2B). ERX-41 decreased tumor growth, as shown by extirpated tumor weights and sizes at the end of study (FIGS. 2C, 2E). Importantly, ERX-41 treatment did not show overt signs of toxicity, as evidenced by no alterations in body weights of the treated mice (FIG. 2D). Further, ERX-41 was able to significantly reduce the growth of D2A1 xenografts in a syngeneic tumor model (FIGS. 2F-H).

The inventors then validated the activity of ERX-41 in four distinct TNBC PDXs established in the mammary fat pad (FIGS. 2I-2P). In each of these tumors, daily oral administration of ERX-41 (10 mg/kg/day) significantly decreased the rates of growth of the tumors in vivo. These data support that ERX-41 has potent activity on TNBC both in vitro and in vivo, without any associated toxicity and anti-estrogenic activity.

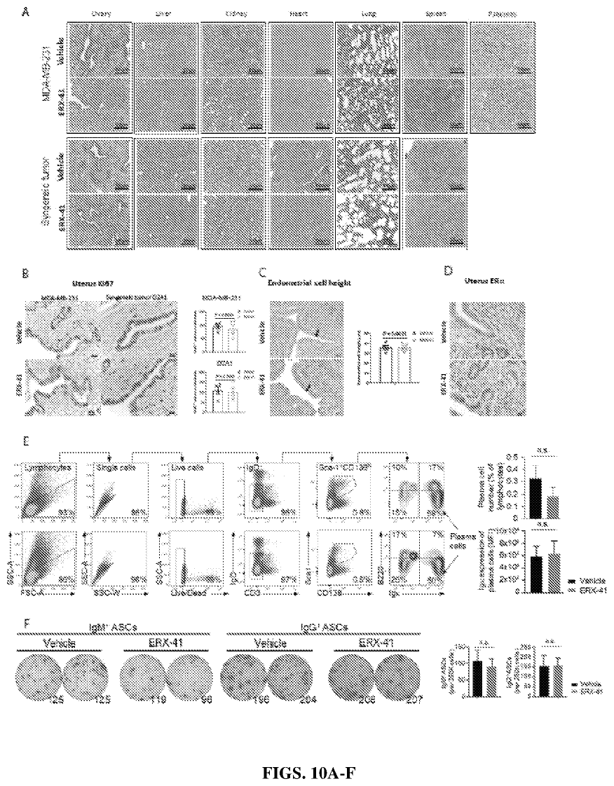

Histologic evaluation of nude mice with MDA-MB-231 xenografts showed no significant changes in the gross histology of multiple organs including the heart, lung, spleen, liver, kidney, ovary and pancreas (FIG. 10A). Similarly, in the syngeneic mice with D2A1 xenografts, no significant histologic changes or immune infiltrates were noted in multiple organs, including the spleen (FIG. 10A), suggesting that ERX-41 was not immunogenic. In addition, ERX-41 did not affect the proliferation index (Ki67, FIG. 10B), the endometrial cell height in the uterus (FIG. 10C) or ERα expression (FIG. 10D): these findings are particularly important since the uterus is the organ most sensitive to estrogenic stimulus and antiestrogenic treatment. Since the bone marrow is at the highest risk for ER stress targeting agents (due to high turnover), the inventors then evaluated the effect of ERX-41 on bone marrow plasma cells by flow cytometry (FIG. 10E) and ELISpot analysis (FIG. 10F). These analyses indicate that ERX-41 treatment did not show any significant effects on either plasma cell numbers, immunoglobulin (Ig) Ig K expression in plasma cells, or the number of IgM or IgG antibody secreting cells (ASCs).

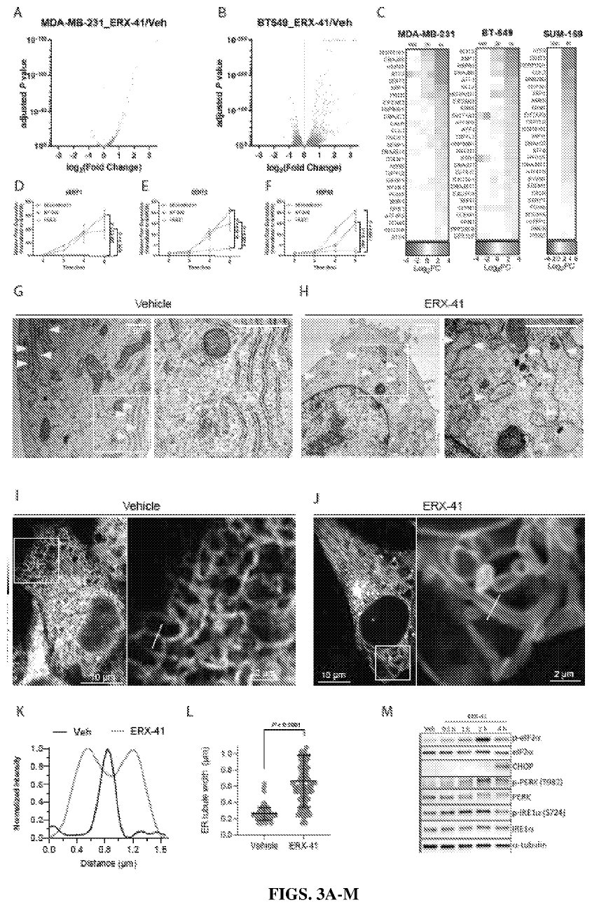

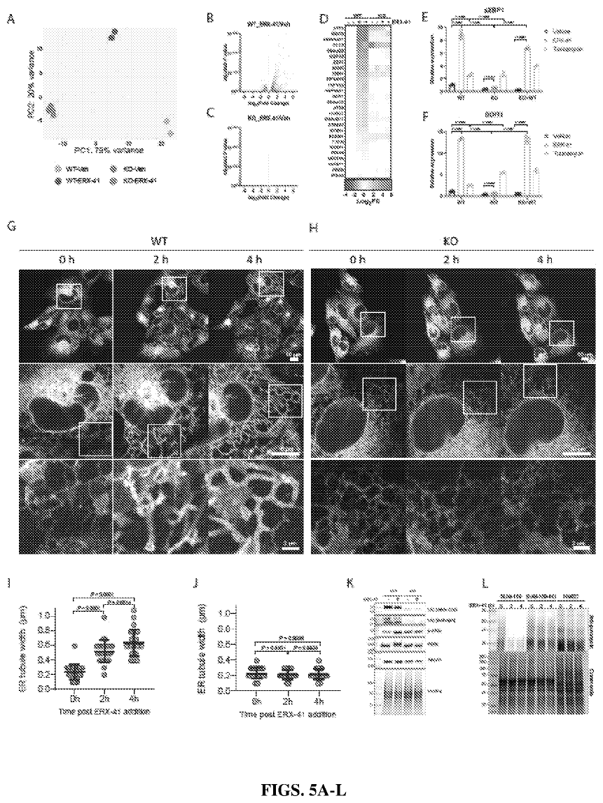

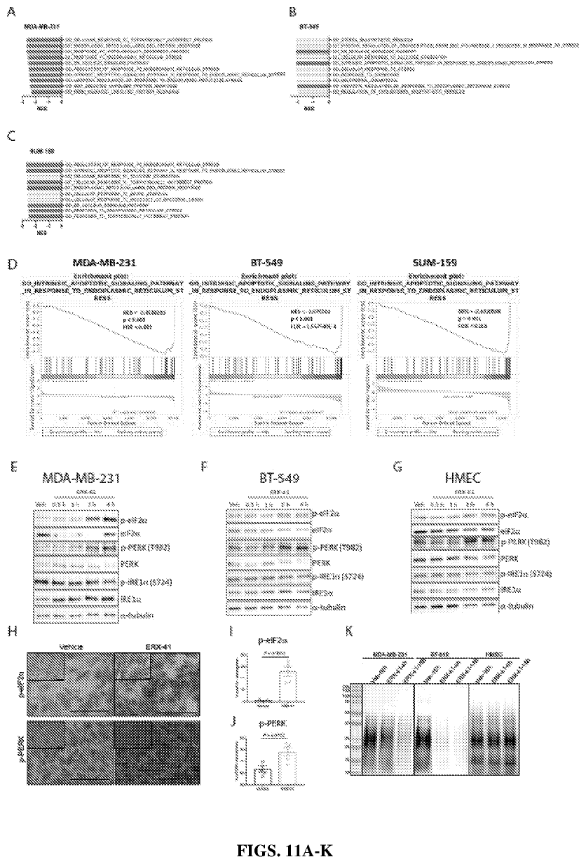

ERX-41 Induces ER stress in TNBC. To understand the mechanism of action of ERX-41 in TNBC, the inventors performed unbiased RNA sequencing studies in three TNBC (SUM-159, MDA-MB-231, and BT549) cells (FIGS. 3A-B, FIG. 5B). Gene Set Enrichment Analysis (GSEA) analyses revealed that the top pathways upregulated after 4 h of ERX-41 treatment in the TNBC cells were related to induction of endoplasmic reticulum (ER) stress and its compensatory unfolded protein response (UPR) pathway (FIGS. 11A-D). Heatmaps show the induction of ER stress and UPR genes in these three TNBC cell lines (FIG. 3C). RT-qPCR validation studies indicate that the mRNA expression of the canonical ER stress genes, heat shock protein 70 A family member 5 (HSPA5) and DNA Damage Inducible Transcript 3 (DDIT3) and UPR stress sensor—the spliced X-box binding protein 1 (sXBP1) is dramatically upregulated (60-80 fold) in TNBC but not in HMEC cells following ERX-41 treatment (FIGS. 3D-F).

Evaluation of ER stress induction was then performed with ultrastructural studies using transmission electron microscopy (TEM). ERX-41 induced dramatic dilation of the ER within 4 h of treatment in TNBC cells (FIGS. 3G-H, ER is outlined by yellow arrowheads). The ER dilation and ER stress in TNBC cells was evident even at 24 h after treatment with ERX-41 (data not shown), suggesting that ERX-41 induces a persistent and prolonged ER stress. The induction of ER dilation by EM was broadly noted in multiple TNBC cells but not in normal cells, indicating that ERX-41 was able to target a vulnerability to induce ER stress in these cancer cells.

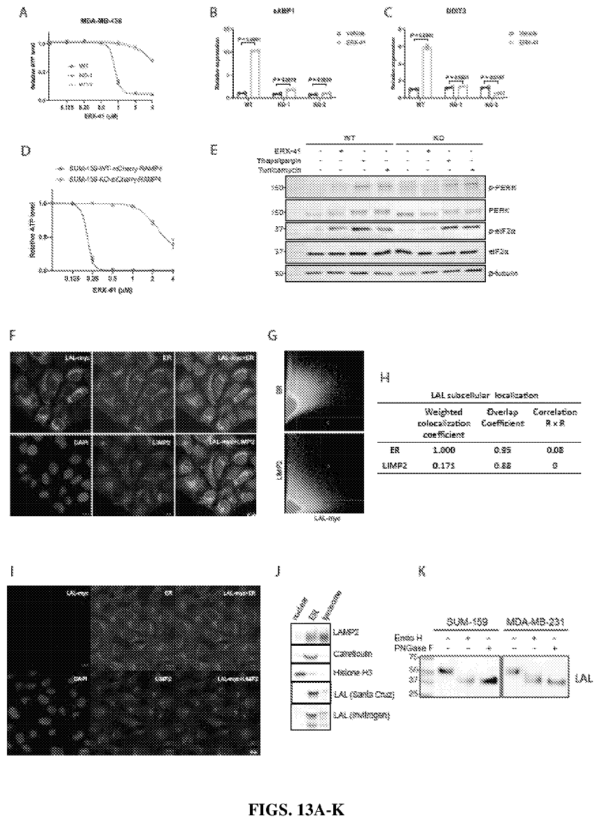

Further ultrastructural validation was obtained by airyscan superresolution microscopy of live SUM-159 TNBC cells stably expressing the ER membrane marker mCherry-RAMP4 (FIGS. 3I-L). The inventors first validated that these stably transfected cell lines are still sensitive to ERX-41 (FIG. 13D). Prior to drug treatment, the peripheral ER network appeared as an intricate network of thin tubules connected by three-way junctions (FIG. 3I) and appear as a single peak on the distance histogram (FIG. 3K). Within 2 h of ERX-41 treatment, the inventors observed a marked disorganization of the peripheral ER network, characterized by a striking elongation and dilation of individual tubules (FIG. 3J, shown as double peaks in FIG. 3K) (mean±S.D. tubule width at 0 h=260±80 nm, 2 h=660±320 nm, n (0 h/2 h)=102/104 tubules, p<0.0001) (quantitated in FIG. 3L). Importantly, since the width of normal ER tubules is reported to be ˜50-100 nm, and thus below the resolving power of airyscan microscopy (100-200 nm), the inventors expect that the effect of ERX-41 on tubule width is even greater than what they report here. These data confirm that ERX-41 induces ER stress in TNBC cells.

The inventors have biochemically validated that ERX-41 induces ER stress and downstream UPR pathways via induction of phosphorylated protein kinase R-like ER kinase (p-PERK), inositol-requiring enzyme 1α (IRE1α, eukaryotic translation initiation factor 2 subunit 1 (eIF2α, and expression of CCAAT-enhancer binding homologous protein (CHOP) in TNBC cells (FIG. 3M, FIGS. 11E-F) but not in HMEC cells (FIG. 11G).

The inventors have validated that a single dose of ERX-41 induces ER stress in TNBC xenografts in vivo, as evidenced by enhanced p-PERK, and p-eIF2α staining in tumor xenograft tissues within 24 h of treatment (FIGS. 11H-J). In contrast, while ERX-41 was detectable in the liver and kidneys within 4 h of oral administration, no significant induction of ER stress was seen in the normal liver or kidney tissues (data not shown). The inability of ERX-41 to induce ER stress in HMEC cells or normal tissues may account for the lack of toxicity seen in mice. In addition, since the inventors' pharmacokinetic studies showed that treated tumor tissues have detectable levels of ERX-41 at 24 h (FIG. 2A), these data indicate that ERX-41 selectively induces ER stress in TNBC.

The functional consequence of ERX-41 induction of ER stress is that the global de novo protein synthesis in multiple TNBC but not HMEC cells is blocked by ERX-41, as shown by western blots for puromycin labeled nascent proteins (FIG. 11K). In addition, the inventors have noted that unlike thapsigargin and tunicamycin, ERX-41 was not able to induce Ca²⁺ flux within the TNBC cells nor induce protein kinase C activity (data not shown). These data suggest that ERX-41 can induce uncompensated ER stress, resulting in shut down of ER function and protein synthesis, leading to cell death.

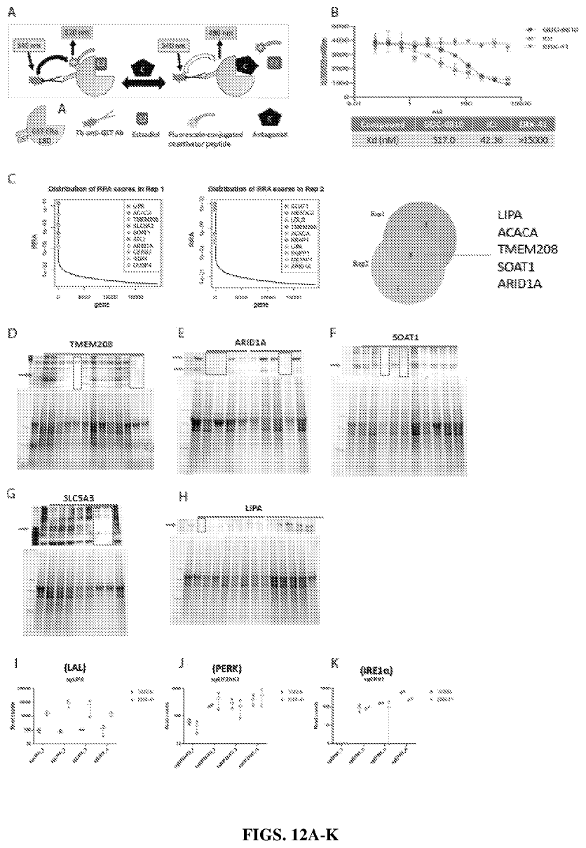

The molecular target of ERX-41 in TNBC is LAL. Since ERX-41 was derived from ERX-11 (which targets ERG), the inventors first wanted to establish that ERX-41 did not interact with ERα. Using a time-resolved measurement of fluorescence with fluorescence resonance energy transfer (TR-FRET) assay, they demonstrated that ERX-41 does not interact with ERG LBD, unlike fulvestrant, selective estrogen receptor degraders like GDC-0810, tamoxifen and ERX-11 (data shown for fulvestrant and GDC-0810) (FIGS. 12A-B). These data strongly suggest that ERX-11 and ERX-41 have distinct molecular targets, which is not surprising given their differential ability to induce ER stress in TNBC.

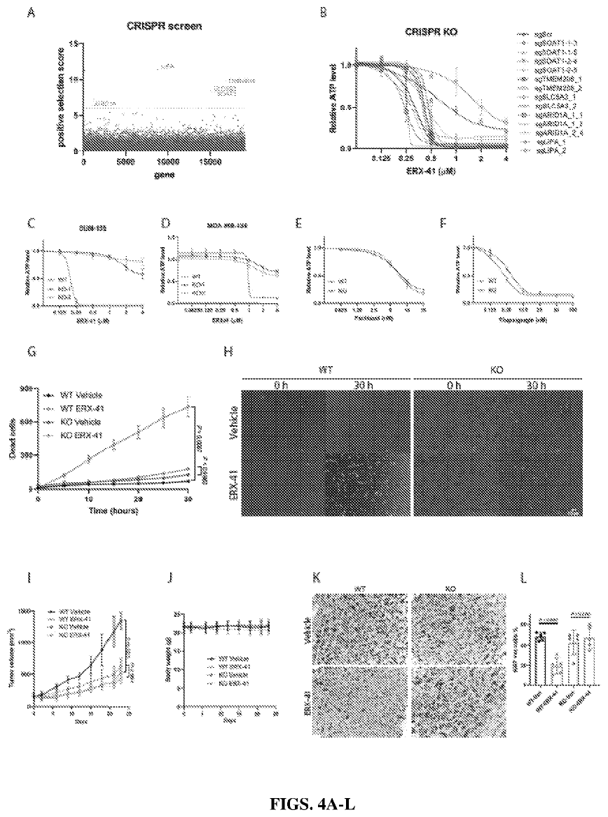

To identify the molecular target of ERX-41, the inventors performed an unbiased CRISPR/Cas9 KO screen in MDA-MB-231 cells. There was significant concordance between two independent experiments of the screen performed at two distinct concentrations of ERX-41 and the top 6 genes from the screen were subject to a secondary screen in MDA-MB-231 cells. (FIG. 4A, FIG. 12C). Knockout clones of 5 of the top 6 genes, IPA, SLC5A3, TMEM208, SOAT1 and ARID1A were generated in multiple TNBC cells and evaluated for response to ERX-41 (FIGS. 12G-4K). Of these, only knockout (KO) of LIPA, the gene of protein LAL, was able to consistently abrogate the cytotoxic response to ERX-41 (FIG. 4B, FIG. 12I). Knockout of individual ER stress/UPR genes like PERK and IRE1α did not affect the ability of ERX-41 to cause cell death, suggesting that multiple ER stress pathways are activated (FIGS. 12J-K). While these data do not rule out a role for the other identified ERX-41 targets, the inventors were able to show that KO of IPA in multiple TNBC cell lines including SUM-159, and MDA-MB-436 was able to alter the response of TNBC cells to ERX-41 (FIGS. 4C-4D) in CellTiter-Glo assays in vitro. The altered response of SUM-159 clones with LIPA KO was specific for ERX-41, as shown by similar responses of SUM-159 parental and LIPA KO clones to thapsigargin (ER stress inducer through its modulation of ER Ca²⁺ levels) and paclitaxel (chemotherapeutic agent) (FIGS. 4E-4F).

Live cell imaging studies confirmed that KO of LIPA altered the response of SUM-159 cells to ERX-41 with dramatic decrease in cell death (quantitation in FIG. 4G, time lapsed pictures in FIG. 4H). The inventors then confirmed that xenografts of SUM-159 with LIPA KO did not respond to i.p., administration of ERX-41, in contrast to parental SUM-159 xenografts which responded significantly to ERX-41 in vivo (FIGS. 4I-J). Importantly, evaluation of the proliferative indices of the xenograft tumors at the time of sacrifice showed a decrease in Ki67 staining in parental SUM-159 xenografts but not in SUM-159 LIPA KO xenografts (FIG. 4K, quantitation in FIG. 4L).

To ascertain that LIPA is associated with ERX-41-induced ER stress, the inventors performed unbiased RNA sequencing studies with and without ERX-41 in the parental and LIPA KO SUM-159 cells. Principal component analyses show that the gene expression profiles in these cells tend to cluster independently (FIG. 5A). While the volcano plot shows significant alteration in the expression of a large number of genes in the parental SUM-159 cells with ERX-41 treatment (FIG. 5B), there are no genes significantly altered in LIPA KO SUM-159 cells upon ERX-41 treatment (FIG. 5C). Evaluation of canonical genes involved in ER stress and UPR response shows induction of these genes by ERX-41 in parental SUM-159 cells but not in the cells with LIPA KO (FIG. 5D). These findings were confirmed by RT-qPCR evaluation showing that ERX-41 induces expression of UPR genes sXBP1 and DDIT3 in parental SUM-159 cells but not in the SUM-159 cells with LIPA KO (FIG. 5E-). These data were validated in the MDA-MB-436 cells with LIPA KO (FIGS. 13A-C).

Ultrastructurally, LIPA KO abrogated the morphological changes in the ER seen at 2 h and 4 h after treatment (FIGS. 5G-H), as evidenced by live cell confocal microscopy. While the changes in ER luminal diameter are more readily apparent with airyscan imaging, the live cell confocal microscopy allows serial evaluation of the same cell over time. As previously noted with airyscan confocal microscopy, ultrastructural changes with increases in ER tubule diameter from ˜200 nm to ˜500 nm at 2 h and ˜620 nm at 4 h were clearly apparent in the parental SUM-159 cells after ERX-41 treatment (quantitated in FIG. 5I). In contrast, no significant changes in ER tubule diameter were noted in LIPA KO SUM-159 cells after ERX-41 treatment (FIG. 5H, quantitated in FIG. 5J). Importantly, the SUM-159 cells with stably transfected mCherry-RAMP4 responded to ERX-41 like the parental SUM-159, showing an IC₅₀˜200 nM in WT cells and ˜4 μM in LIPA KO cells (FIG. 13D).

At the protein level, western blotting reveals that ERX-41 activates PERK (noted by upshifting of PERK on western) and induces peIF2α in parental SUM-159 cells but not in the SUM-159 cells with LIPA KO (FIG. 5K). Importantly, the inducibility of UPR genes in SUM-159 cells with LIPA KO with known ER stress inducers thapsigargin (inhibitor of ER Ca²⁺ ATPase) or tunicamycin (blocks N-liked glycosylation) is preserved (FIGS. 5E-F, FIG. 13E) and only the ability of ERX-41 to induce ER stress in these cells is compromised. Consequently, ERX-41 blocks de novo protein synthesis in the parental SUM-159 cells but not in the SUM-159 cells with LIPA KO and HMEC cells (FIG. 5L).

Subcellular localization of LAL in the ER. The known function of LAL protein as lysosomal acid lipase relates to its subcellular localization in the lysosome. To evaluate the subcellular localization of LAL protein in TNBC cells, the inventors performed co-immunofluorescence with both ER or lysosomal markers in SUM-159 cells with overexpressed myc-tagged wild type LAL (FIG. 13F). They used highly specific myc antibody to indicate LAL localization. The specificity of myc antibody was confirmed by negative staining in parental SUM-159 cells which have no myc-tagged LAL expression (FIG. 13I). The inventors noted a high colocalization of LAL protein with the ER tracker with a weighted colocalization factor of 1.0 (FIGS. 13F-H). In contrast, only some of the LAL protein colocalized with the lysosomal marker LIMP2, with a weighted colocalization factor of 0.17 (FIGS. 13F-H). These data were further supported by biochemical evaluation of subcellular fractions of SUM-159 cells (FIG. 13J), which reveals an enrichment of LAL protein in the ER-enriched subcellular fraction. In addition, the sensitivity of glycosylated LIPA to endoglycosidase H (Endo H) and Peptide-N-Glycosidase F (PNGase F) cleavage further supports its localization in the ER⁸ (FIG. 13K). If the glycosylation occurred in the Golgi, then the glycosylated LIPA would only be sensitive to PNGase F but not to Endo H cleavage. Since the role of LIPA in the ER has not been previously described, these data support the inventors' central finding that LIPA plays a novel role in ER homeostasis. Taken together, these data suggest a previously uncharacterized function for LIPA in the ER that is targeted by ERX-41 and that induces ER stress in TNBC.

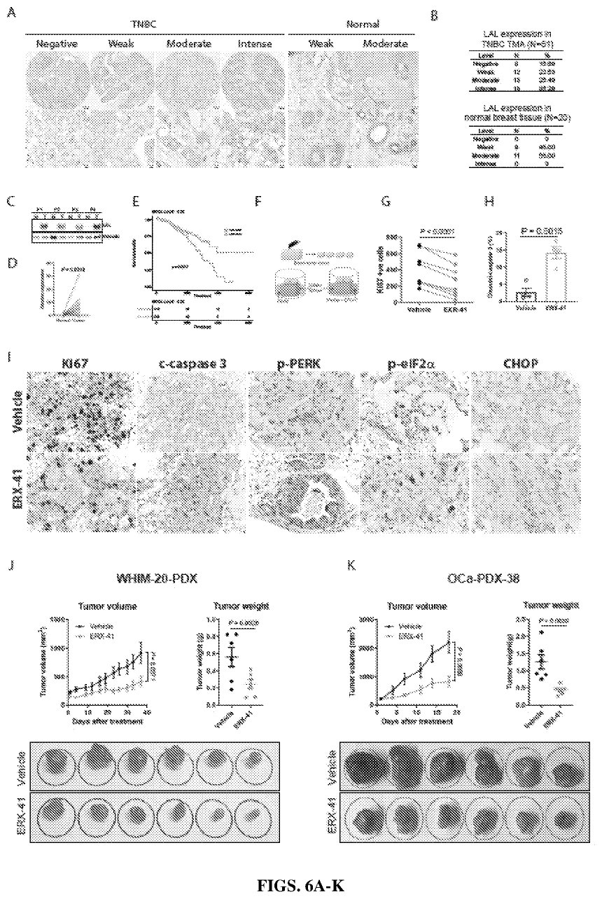

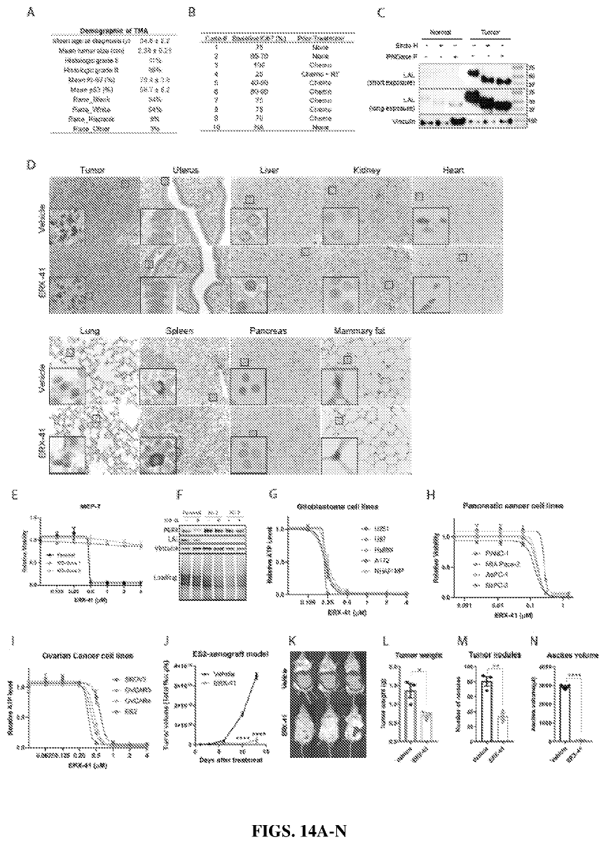

LAL is a viable molecular target in TNBC. The inventors had noted that LAL is widely expressed in multiple TNBC cell lines. To confirm that LAL is a viable target for ERX-41 in TNBC, they evaluated LAL protein expression using a TNBC tissue microarray (patient demographics and prior treatment tabulated in FIG. 14A). The inventors found that more than 80% of primary untreated TNBC tumors had significant and detectable LAL protein expression (FIGS. 6A-B). In contrast, normal breast tissue has lower LAL expression (FIGS. 6A-B). Protein expression was noted to be higher in TNBC tumors than adjacent normal breast tissue (FIGS. 6C-D). Importantly, the glycosylation of LAL in TNBC tumors is similar to that observed in TNBC cell lines (FIG. 14C). Analysis of publicly available datasets (TCGA) indicated that high LAL expression correlated with significantly worse overall survival outcomes in breast cancer patients (FIG. 6E). These data suggest that LAL is a viable molecular ERX-41 target for translation in TNBC.

The minimal toxicity of ERX-41 in vivo prompted the evaluation of LAL protein expression in normal mouse organs with IHC evaluation. The inventors noted that LAL protein expression in multiple mouse tissues, including the uterus, liver, kidney, heart, lung, spleen, pancreas, and mammary fat pad is much lower than in the tumor (FIG. 14D). Among normal tissues, the expression level is lowest in the heart, pancreas and mammary fat pad, intermediate in the liver and kidney and highest in the spleen. Given the role of the spleen in the lymphatic system, the inventors' prior analyses showing that ERX-41 had no effect on plasma cells is of relevance. These data suggest that the enhanced expression of LAL in tumors may account for their sensitivity to ERX-41.

To ascertain that ERX-41 would have activity against primary TNBC tumors, the inventors leveraged their prior significant experience with the ex vivo patient derived explant (PDE) cultures (FIG. 6F).^{7,9-11} The PDE cultures, maintains the native tissue architecture and better recapitulates the heterogeneity of human TNBC in a laboratory setting (patient characteristics detailed in FIG. 14B). The inventors have noted that ERX-41 had significant activity, as evidenced by decreased proliferation (Ki67 staining) and increased apoptosis (cleaved caspase 3 staining) of primary TNBC PDEs (FIGS. 6G-I). Importantly, evaluation of these explants with immunohistochemical evaluation of UPR pathway markers shows enhanced pPERK, CHOP (protein product of DDIT3 gene) and peIF2a staining within 24 h after treatment with ERX-41 (FIG. 6I). Importantly, all these TNBC tumors had significant LAL expression as noted by immunohistochemistry (data not shown). These data further validate the on-target activity and potential utility of ERX-41, specifically in patients who would receive the drug.

LAL is a viable molecular target of ERX-41 in other cancers. Since LAL expression was enhanced in TNBC and TNBC have a high basal level of ER stress, the inventors postulated that ERX-41 would have activity in other tumors with high basal levels of ER stress, such as ERα+ breast, glioblastoma, pancreatic and ovarian cancers. Initial evaluation in cell lines confirmed their high expression of LIPA (data not shown) and their responsiveness to ERX-41 in vitro (FIGS. 14E-I). Importantly, the inventors have shown that knockdown of LAL expression in these cell lines using CRISPR abrogated the ability of ERX-41 to induce ER stress (FIG. 14F shown for ERα+ MCF-7 cells). They have also confirmed that ERX-41 has activity against both ERα+ breast PDX (WHIM-20) (FIG. 6J) and ovarian cancer cell line xenograft (ES2) (FIGS. 14J-N) and PDX (OCa-PDX-38) (FIG. 6K). These data indicate that ERX-41 has activity against multiple tumors by targeting LAL and inducing ER stress.

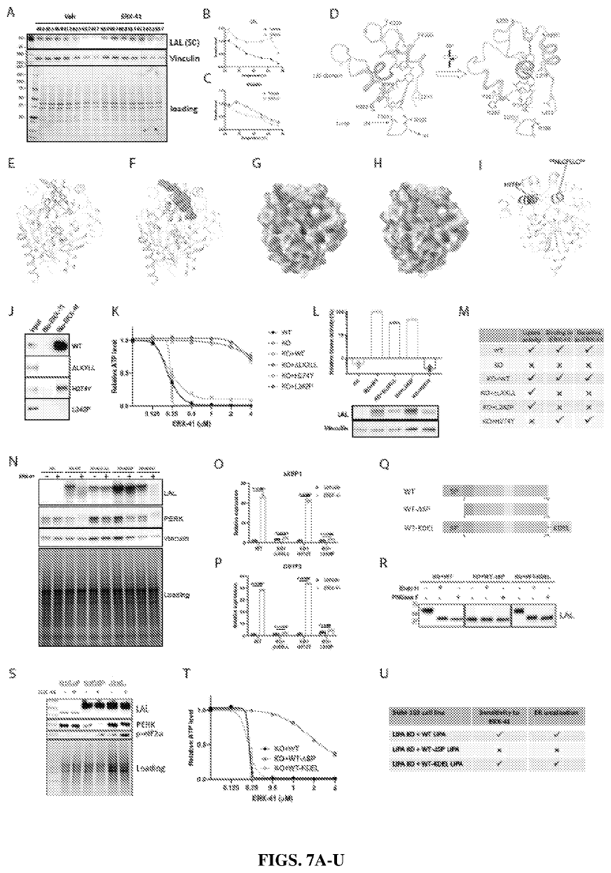

ERX-41 interacts with LAL through residues in the LXXLL motif and its activity is independent of LAL lipase activity. The inventors used cellular thermal shift assays to confirm that ERX-41 binds to the protein product of the LIPA gene, lysosomal acid lipase (LAL) protein. These studies indicate that ERX-41 was able to stabilize the thermal stability of LAL within the cell and shifted the thermal sensitivity, suggesting that ERX-41 binds to LAL (FIGS. 7A-B). In contrast, ERX-41 did not affect vinculin thermal stability (FIG. 7C). Subsequent assays confirm binding of LAL to ERX-41 (FIG. 7J) and indicate ERX-41 but not ERX-11 binds to LAL in the cellular context of TNBC cells.

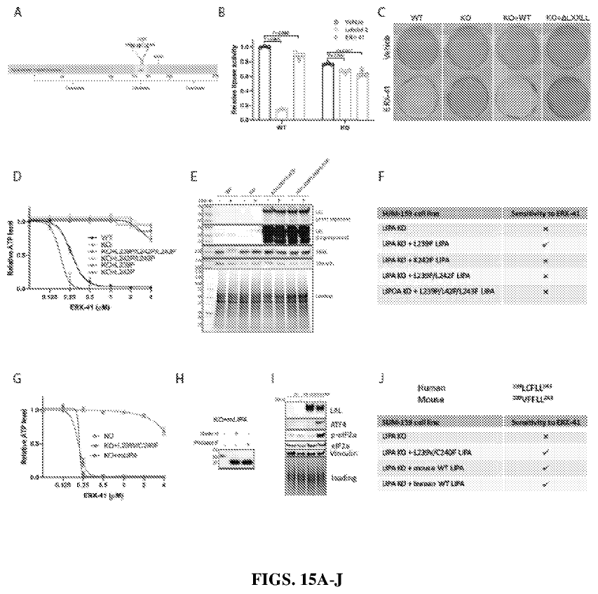

To study how ERX-41 interacts with LAL, the inventors used in silico molecular docking simulation to evaluate potential binding sites of ERX-41 on LAL (FIGS. 7D-I). They noted that LIPA has a single ²³⁹LXXLL²⁴³ motif and that ERX-41 (shown in green, FIG. 7D-I) could potentially interact with this LXXLL motif which was noted to be in the lid region of LAL (LXXLL motif shown in orange (FIGS. 7D-I, FIG. 15A). Structurally, the critical domain of lipase activity of LAL appeared to be spatially distinct from the LXXLL motif: previous studies have identified a LAL point mutation H274Y in LAL helix 13, that is known to abrogate the known lipase function of LIPA (FIG. 7I).¹² Further, ERX-41 showed no inhibition of lipase activity of LIPA: in contrast, Lalistat 2 (specific inhibitor of LAL lipase activity) attenuated lipase activity in SUM-159 cells (FIG. 15B). The specificity of Lalistat 2 for LAL is confirmed by its lack of inhibitory activity on lipase function (from other lipases) in LIPA-KO cells.^{13,14}

To study the effect of these LIPA domains, the inventors synthesized LIPA plasmid constructs under a constitutive promoter, including wild type LIPA (WT-LIPA), H274Y mutant LIPA (H274Y MT-LIPA), ΔLXXLL mutant LIPA (deletion of the ²³⁸NLCFLLC²⁴⁴), and the L242P mutant LIPA (point mutation of the second L in the LXXLL motif). Evaluation of the lipase activity of these constructs confirmed that the WT-LIPA, ΔLXXLL MT-LIPA and L242P MT-LIPA had lipase activity while the H274Y MT-LIPA did not have lipase activity (FIG. 7L). The inventors then confirmed direct interaction of ERX-41 with LIPA using biotinylated ERX-41 pulldown which indicated interaction between ERX-41 and proteins encoded by WT-LIPA and H274Y MT-LIPA but not ΔLXXLL MT-LIPA or L242P MT-LIPA (FIG. 7J). Reconstitution of WT-LIPA (KO+WT) in SUM-159 cells with LIPA-KO restored sensitivity to ERX-41, with an ICso of 250 nM (FIG. 7K). Interestingly, reconstitution of the lipase-incompetent H274Y MT-LIPA (KO+H274Y) also restored sensitivity to ERX-41 (FIG. 7K). In contrast, reconstitution of the lipase-competent ΔLXXLL MT-LIPA (KO+ΔLXXLL) did not restore sensitivity to ERX-41, suggesting that the LXXLL motif is critical for LAL-ERFX-41 interaction (FIG. 7K). Detailed mutational evaluation of the ²³⁹LXXLL²⁴³ domain indicates that the leucine at the 242 position is critical for ERX-41 binding and activity (FIGS. 7J-M, FIGS. 15C-E). In contrast the leucine at the 239 position is not critical for ERX-41 binding or activity (FIG. 15D). These data are validated by the inability of ERX-41 to affect proliferation or induce ER stress (shown by PERK activation) in SUM-159 clones with reconstitution of lipase-competent LXXLL motif single mutations (KO+L242P), double mutations (KO+L239P/L242P) or triple mutations (KO+L239P/L242P/L243P) (FIGS. 15C-F). These findings are further supported by the ability of WT-LIPA and H274Y MT-LIPA but not ΔLXXLL MT-LIPA or L242P MT-LIPA to restore the ability of ERX-41 to induce ER stress at the protein level (shown by activation of PERK in FIG. 7N) and UPR genes at the RNA level (sXBP1 and DDIT3 levels in FIGS. 7O-P) in SUM-159 cells with LIPA-KO. These data taken together indicate that ERX-41 interacts with LAL through residues in its LXXLL domain and its ability to induce ER stress and cell death in TNBC is independent of the lipase activity of LAL (Tabulated in FIG. 7M).

The inventors then evaluated if LAL localization to the ER is critical for LAL function using additional LAL recombinants with altered subcellular localization (FIGS. 7Q-U). They noted that a recombinant LIPA cDNA lacking the signal peptide is neither localized to the ER, does not get glycosylated (FIG. 7R) nor restore the ability of ERX-41 to cause ER stress (FIG. 7S) or affect cell proliferation (FIG. 7T). In contrast, a recombinant LIPA cDNA with an added KDEL sequence at the C-terminus is localized to the ER, is glycosylated (FIG. 7R) and restores the ability of ERX-41 to cause ER stress (FIG. 7S) and affect cell proliferation (FIG. 7T). These data indicate the ER localization of LAL is critical for cellular responsiveness to ERX-41 (FIG. 7U).

Finally, since the mouse LAL has a slightly different LXXLL sequence (VFFLL), the inventors confirmed that both this altered sequence in the context of human LIPA or the entire mouse LIPA cDNA is glycosylated, could bind to ERX-41 and restore responsiveness (ER stress and cell death) to ERX-41 in SUM-159 LIPA KO cells (FIGS. 15G-J). In conjunction with earlier findings that a tumor cell line of murine origin (D2A1) is sensitive to ERX-41 (FIGS. 2F-H), these data indicate that the non-toxic characteristics of ERX-41 in mice cannot be attributed to species differences in LAL sequences.

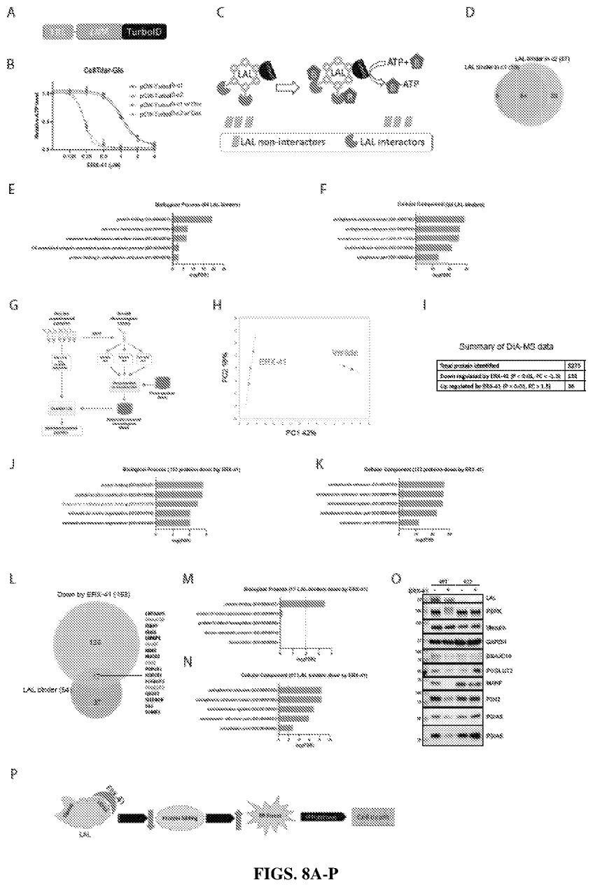

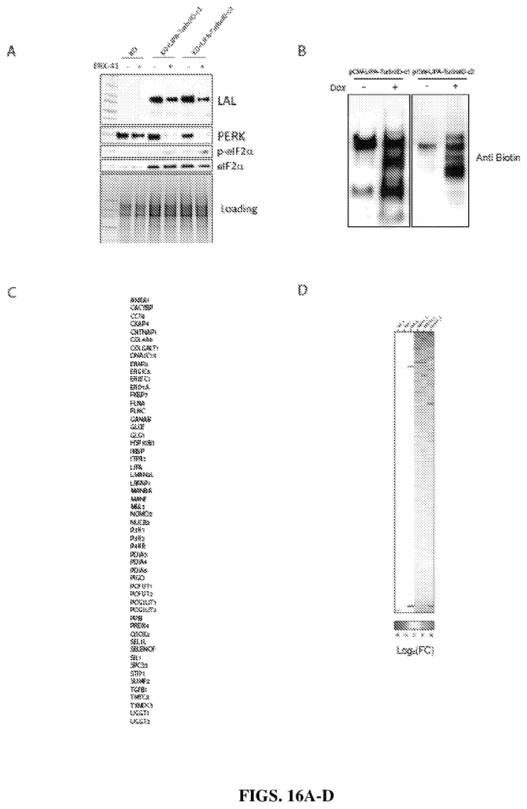

Interaction of ERX-41 with LAL alters expression of proteins involved in protein folding. To molecularly characterize how the interaction of ERX-41 with the LAL protein causes ER stress, the inventors first decided to define the LAL interactome using a recombinant LAL fused to TurboID (FIG. 8A). Importantly, this recombinant LAL was able to reconstitute sensitivity to ERX-41 in SUM-159 LIPA KO cells in Cell-Titer-Glo studies (FIG. 8B) and restore ability of ERX-41 to cause ER stress (FIGS. 16A-B). LAL interactors were biotinylated, isolated by streptavidin pulldown and identified by unbiased mass-spectrometric analyses by two separate replicates (FIGS. 8C-D). Gene ontogeny analysis of the top 54 LAL interacting proteins (FIG. 16C) revealed that four of the top five cellular processes involved in critical ER protein maturation functions, such as protein folding in the ER (FIG. 8E). In addition, the cellular component analyses indicate that the LAL binders were most commonly localized to the ER (FIG. 8F). These data are completely congruent with the inventors' prior and novel findings that LAL is involved in ER functions.

The inventors next used a global data-independent acquisition mass spectrometry (DIA-MS), a next generation proteomic methodology that generates digital proteome maps (FIG. 8G). Using unbiased quantitative DIA-MS approach, they identified that short-term (6 h) ERX-41 treatment significantly affected the expression of 189 cellular proteins (heatmap in FIG. 16D), including 153 down-regulated and 36 up-regulated proteins (see principal component analyses proteins in FIGS. 8H-I). Gene ontology analysis of the 153 ERX-41 downregulated proteins revealed that three of the top five cellular processes involved in critical ER protein maturation functions, such as protein folding in the ER (FIG. 8J) and that proteins were most commonly localized to the ER (FIG. 8K).

To further refine their studies, the inventors combined the data from both unbiased proteomic approaches to identify a core set of proteins that were both LAL binders and affected by ERX-41 treatment (FIG. 8L). Again, gene ontogeny analyses of these 17 proteins confirm the importance of protein folding (FIG. 8M) and localization to the ER (FIG. 8N). The inventors then validated the LAL dependence of ERX-41 activity using both LAL expressing and LAL knockout cells: Western blots of 6 of these proteins showed that ERX-41 activity affects the expression of these proteins in LAL expressing but not in LAL knockout cells (FIG. 8O). These data support the model that ERX-41 binding to LAL affects the expression of several ER localized proteins involved in ER protein maturation and induction of ER stress.

Taken together, the findings in this disclosure indicate that ERX-41 binds to LAL through residues in the LXXLL domain in the ER, and disrupts proteins involved in protein-folding in the ER causing significant ER stress/UPR, leading to cell death in TNBC cells. The lipase function of LAL is not affected by ERX-41 nor critical for ERX-41 activity. These findings are modeled in FIG. 8P.

### Example 4

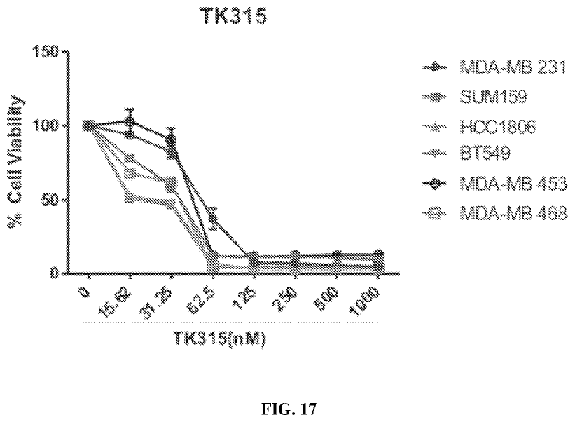

The ERX-315 compound, described herein as the compound of Formula II, was tested for its effectiveness in treating cell culture models of cancer. As shown in FIG. 17, the ERX-315 compound was effective in each cell line tested.

The inventors have conducted several studies at UTHSCSA using preclinical murine Xenograft and Patient derived xenografts (PDX) examining the efficacy of compound TK315 (ERX-315). The results are given below.

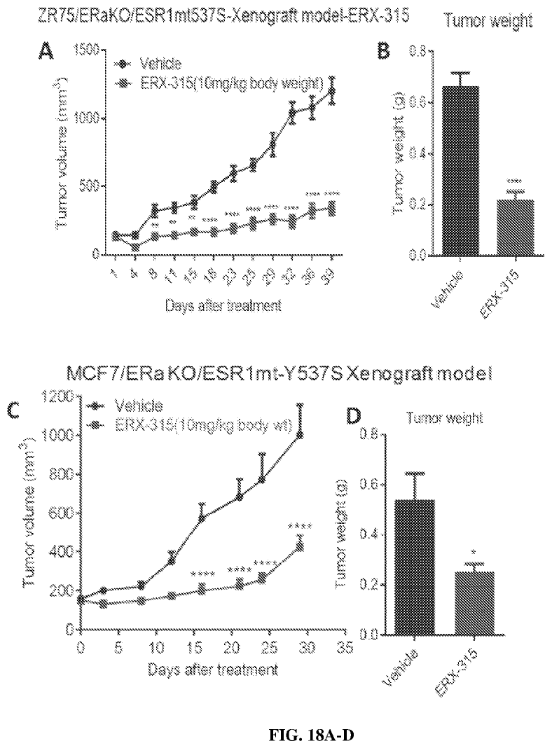

Oral administration of TK315 (ERX-315) in captisol formulation showed potent activity against both MCF7-MT ESRJ ZR-75 and ZR75-MT Y537S ERα expressing therapy resistant BC xenograft models but no effect on mouse liver or body weight (FIGS. 18A-D). Histologic evaluation of the tumors showed dramatically decreased K167 proliferation indices in these tumors. Importantly, the lack of immune antibody infiltrates in the spleen, lymph nodes, kidney, or liver of the syngeneic D2A1 tumors with treated with ERX-315 suggested that ERX-315 is potent, not immunogenic and can be safely administered orally.

PDX models recapitulate the structural complexity and individual heterogeneity of human BC (primary tumor samples), therefore, studies with these models will establish an incontrovertible basis for clinical translation.

### Example 5

Compositions comprising compounds of Formula I (as disclosed herein) are effective in treating a cancer.

In some cases, a subject (a human subject) is treated for a cancer characterized by having increased activity of Lysosomal lipase A (LIPA). In this method, the subject having a cancer with elevated LIPA expression is administered a therapeutically-effective amount of a composition comprising a compound of Formula I:

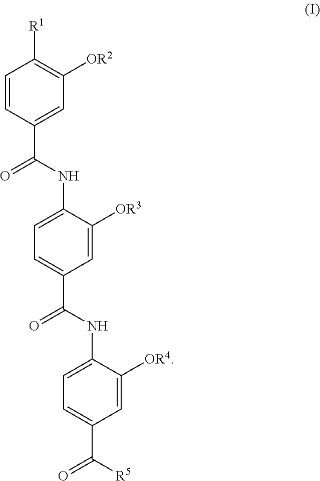

In other cases, a subject (a human subject) is treated for a cancer once the subject has been tested for the presence of elevated LIPA expression. In this method, a sample from the subject is tested for the presence of elevated LIPA expression and, if elevated LIPA expression is detected, the subject is administered a therapeutically-effective amount of a composition comprising a compound of Formula I:

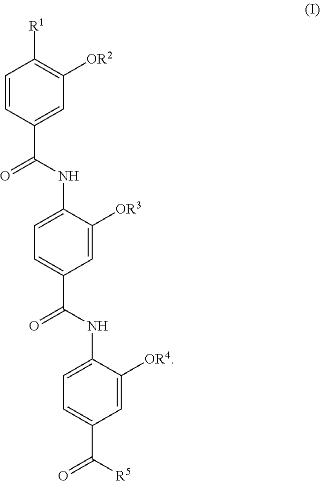

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In additional cases, activity of Lysosomal lipase A (LIPA) is inhibited in a subject having a cancer. In this method, the subject is administered a therapeutically-effective amount of a composition comprising a compound of Formula I:

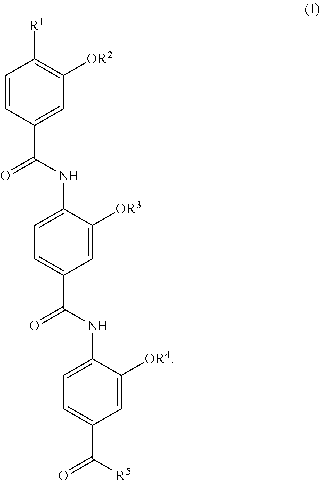

The compound of Formula I may have the following embodiments: R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein: R^{1a} is aralkyl_{(C<18)}, substituted aralkyl_{(C<18)}, or the side chain of a canonical amino acid; R², R³, and R⁴ are each independently alkyl_{(C<12)}, substituted alkyl_{(C<12)}, aralkyl_{(C<18)}, or substituted aralkyl_{(C<18)}; and R⁵ is —OR^{5a} or —NHR^{5b}, wherein: R^{5a} is alkyl_{(C<12)} or substituted alkyl_{(C<12)}; R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or a group of the formula:

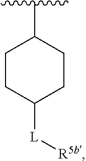

wherein; L is —CO₂— or —C(O)NR^{L}, wherein: R^{L} hydrogen, alkyl_{(C<12)}, or substituted alkyl_{(C<12)}; R^{5b′} is aryl_{(C<12)}, aralkyl_{(C<18)}, heteroaryl_{(C<12)}, heteroaralkyl_{(C<18)}, or a substituted version of any of these groups.

In embodiments, the compound of Formula I inhibits LIPA activity when contacted to the LXXLL motif on LIPA.

In several embodiments, the compound is a pharmaceutically acceptable salt of Formula I.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In various embodiments, the administering comprises intravenous, intra-arterial, intra-tumoral, subcutaneous, topical, or intraperitoneal administration.

In some embodiments, the administering comprises local, regional, systemic, or continual administration.

In several embodiments, the method further comprises providing to said subject a second anti-cancer therapy. In some cases, said second anti-cancer therapy is surgery, chemotherapy, radiotherapy, hormonal therapy, toxin therapy, immunotherapy, and cryotherapy. The said second anti-cancer therapy may be provided prior to administering said composition, the second anti-cancer therapy may be provided after administering said composition, and/or the second anti-cancer therapy may be provided contemporaneous with said composition.

In embodiments, the said composition is administered daily, e.g., daily for 7 days, 2 weeks, 3 weeks, 4 weeks, one month, 6 weeks, 8 weeks, two months, 12 weeks, or 3 months.

In various embodiments, the composition is administered weekly, e.g., weekly for 2 weeks, 3 weeks, 4 weeks, 6 weeks, 8 weeks, 10 weeks, or 12 weeks.

In some embodiments, the compound or composition is administered in an amount sufficient to induce endoplasmic reticulum stress, in an amount sufficient to inhibit activity of LIPA, and/or in an amount sufficient to reduce protein synthesis.

### Example 6

Compositions comprising the compound of Formula II (as disclosed herein) are effective in treating a cancer.

In some cases, a subject (a human subject) is treated for a cancer characterized by having increased activity of Lysosomal lipase A (LIPA). In this method, the subject having a cancer with elevated LIPA expression is administered a therapeutically-effective amount of a composition comprising the compound of Formula II:

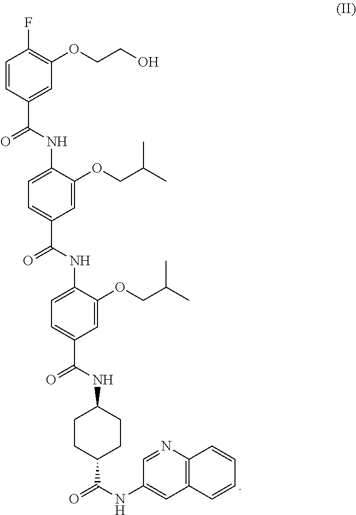 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

In other cases, a subject (a human subject) is treated for a cancer once the subject has been tested for the presence of elevated LIPA expression. In this method, a sample from the subject is tested for the presence of elevated LIPA expression and, if elevated LIPA expression is detected, the subject is administered a therapeutically-effective amount of a composition comprising the compound of Formula II:

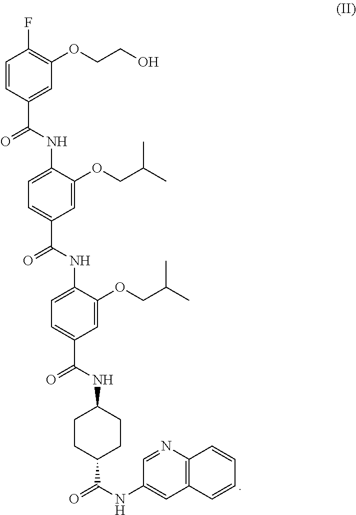 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In additional cases, activity of Lysosomal lipase A (LIPA) is inhibited in a subject having a cancer. In this method, the subject is administered a therapeutically-effective amount of a composition comprising the compound of Formula II:

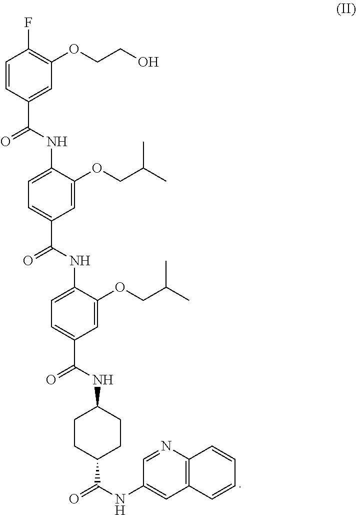 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

In embodiments, the compound of Formula II inhibits LIPA activity when contacted to the LXXLL motif on LIPA.

In several embodiments, the compound is a pharmaceutically acceptable salt of Formula II.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In various embodiments, the administering comprises intravenous, intra-arterial, intra-tumoral, subcutaneous, topical, or intraperitoneal administration.

In some embodiments, the administering comprises local, regional, systemic, or continual administration.

In several embodiments, the method further comprises providing to said subject a second anti-cancer therapy. In some cases, said second anti-cancer therapy is surgery, chemotherapy, radiotherapy, hormonal therapy, toxin therapy, immunotherapy, and cryotherapy. The said second anti-cancer therapy may be provided prior to administering said composition, the second anti-cancer therapy may be provided after administering said composition, and/or the second anti-cancer therapy may be provided contemporaneous with said composition.

In embodiments, the said composition is administered daily, e.g., daily for 7 days, 2 weeks, 3 weeks, 4 weeks, one month, 6 weeks, 8 weeks, two months, 12 weeks, or 3 months.

In various embodiments, the composition is administered weekly, e.g., weekly for 2 weeks, 3 weeks, 4 weeks, 6 weeks, 8 weeks, 10 weeks, or 12 weeks.

In some embodiments, the compound or composition is administered in an amount sufficient to induce endoplasmic reticulum stress, in an amount sufficient to inhibit activity of LIPA, and/or in an amount sufficient to reduce protein synthesis.

### Example 7

Compositions comprising the compound of Formula III (as disclosed herein) are effective in treating a cancer.

In some cases, a subject (a human subject) is treated for a cancer characterized by having increased activity of Lysosomal lipase A (LIPA). In this method, the subject having a cancer with elevated LIPA expression is administered a therapeutically-effective amount of a composition comprising the compound of Formula III:

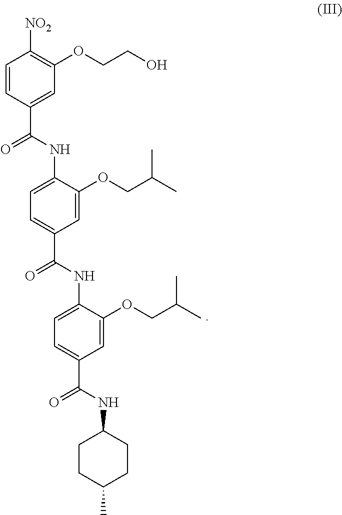 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

In other cases, a subject (a human subject) is treated for a cancer once the subject has been tested for the presence of elevated LIPA expression. In this method, a sample from the subject is tested for the presence of elevated LIPA expression and, if elevated LIPA expression is detected, the subject is administered a therapeutically-effective amount of a composition comprising the compound of Formula III:

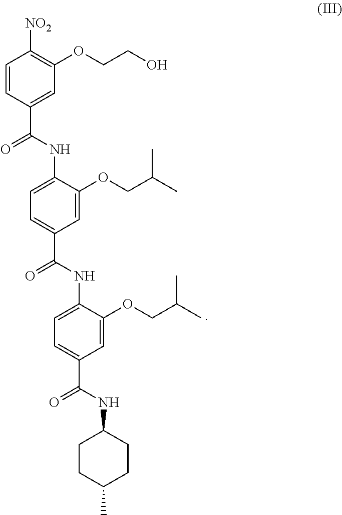 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In additional cases, activity of Lysosomal lipase A (LIPA) is inhibited in a subject having a cancer. In this method, the subject is administered a therapeutically-effective amount of a composition comprising the compound of Formula III:

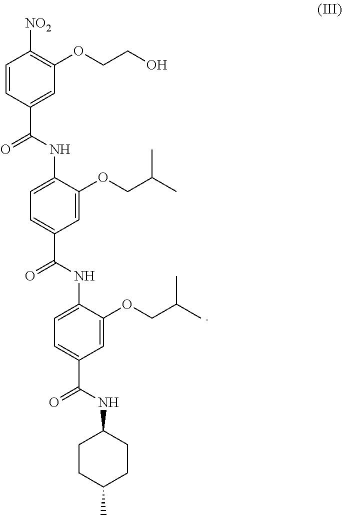 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

In embodiments, the compound of Formula III inhibits LIPA activity when contacted to the LXXLL motif on LIPA.

In several embodiments, the compound is a pharmaceutically acceptable salt of Formula III.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In various embodiments, the administering comprises intravenous, intra-arterial, intra-tumoral, subcutaneous, topical, or intraperitoneal administration.

In some embodiments, the administering comprises local, regional, systemic, or continual administration.

In several embodiments, the method further comprises providing to said subject a second anti-cancer therapy. In some cases, said second anti-cancer therapy is surgery, chemotherapy, radiotherapy, hormonal therapy, toxin therapy, immunotherapy, and cryotherapy. The said second anti-cancer therapy may be provided prior to administering said composition, the second anti-cancer therapy may be provided after administering said composition, and/or the second anti-cancer therapy may be provided contemporaneous with said composition.

In embodiments, the said composition is administered daily, e.g., daily for 7 days, 2 weeks, 3 weeks, 4 weeks, one month, 6 weeks, 8 weeks, two months, 12 weeks, or 3 months.

In various embodiments, the composition is administered weekly, e.g., weekly for 2 weeks, 3 weeks, 4 weeks, 6 weeks, 8 weeks, 10 weeks, or 12 weeks.

In some embodiments, the compound or composition is administered in an amount sufficient to induce endoplasmic reticulum stress, in an amount sufficient to inhibit activity of LIPA, and/or in an amount sufficient to reduce protein synthesis.

### Example 8

Compositions comprising the compound of Formula IV (as disclosed herein) are effective in treating a cancer.

In some cases, a subject (a human subject) is treated for a cancer characterized by having increased activity of Lysosomal lipase A (LIPA). In this method, the subject having a cancer with elevated LIPA expression is administered a therapeutically-effective amount of a composition comprising the compound of Formula IV:

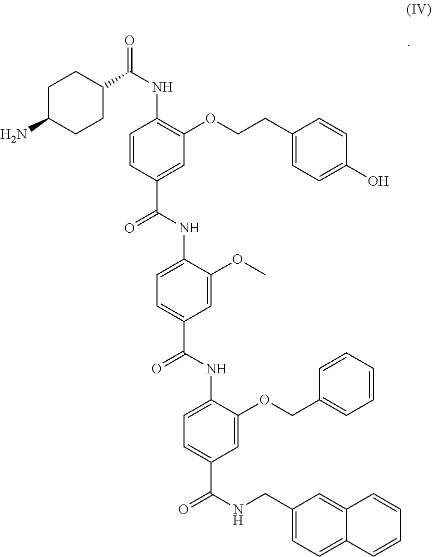

In other cases, a subject (a human subject) is treated for a cancer once the subject has been tested for the presence of elevated LIPA expression. In this method, a sample from the subject is tested for the presence of elevated LIPA expression and, if elevated LIPA expression is detected, the subject is administered a therapeutically-effective amount of a composition comprising the compound of Formula IV:

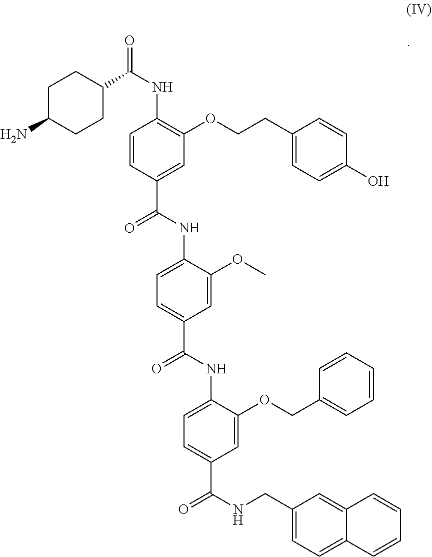

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In additional cases, activity of Lysosomal lipase A (LIPA) is inhibited in a subject having a cancer. In this method, the subject is administered a therapeutically-effective amount of a composition comprising the compound of Formula IV:

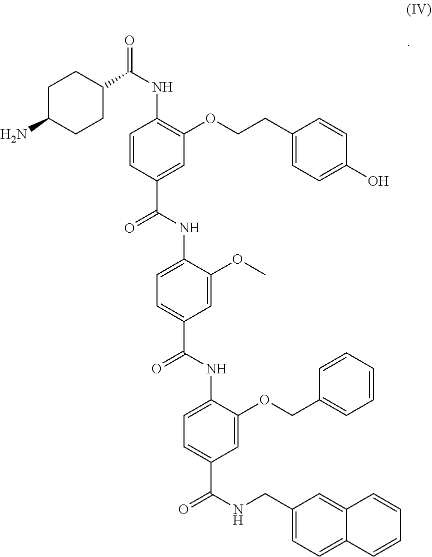

In embodiments, the compound of Formula IV inhibits LIPA activity when contacted to the LXXLL motif on LIPA.

In several embodiments, the compound is a pharmaceutically acceptable salt of Formula IV.

In embodiments, the cancer is breast cancer (e.g., a triple negative breast cancer, an estrogen receptor-positive cancer, or an estrogen receptor-negative cancer) ovarian cancer, pancreatic cancer, or brain cancer (e.g., a glioblastoma).

In various embodiments, the administering comprises intravenous, intra-arterial, intra-tumoral, subcutaneous, topical, or intraperitoneal administration.

In some embodiments, the administering comprises local, regional, systemic, or continual administration.

In several embodiments, the method further comprises providing to said subject a second anti-cancer therapy. In some cases, said second anti-cancer therapy is surgery, chemotherapy, radiotherapy, hormonal therapy, toxin therapy, immunotherapy, and cryotherapy. The said second anti-cancer therapy may be provided prior to administering said composition, the second anti-cancer therapy may be provided after administering said composition, and/or the second anti-cancer therapy may be provided contemporaneous with said composition.

In embodiments, the said composition is administered daily, e.g., daily for 7 days, 2 weeks, 3 weeks, 4 weeks, one month, 6 weeks, 8 weeks, two months, 12 weeks, or 3 months.

In various embodiments, the composition is administered weekly, e.g., weekly for 2 weeks, 3 weeks, 4 weeks, 6 weeks, 8 weeks, 10 weeks, or 12 weeks.

In some embodiments, the compound or composition is administered in an amount sufficient to induce endoplasmic reticulum stress, in an amount sufficient to inhibit activity of LIPA, and/or in an amount sufficient to reduce protein synthesis. FIGS. 19A-B: MTT assays showing the effect of TK-342 (Formula IV) on cell viability of SUM-159 cells (WT) and SUM-159 cells with LIPA KO and LIPA OE, as well as cell viability of SUM-159 cells (WT) and SUM-159 cells with SOAT1. Data shown are the means of ±SEM performed in triplicate wells.

### Example 9

The ability of a composition comprising the compound of Formula II (as disclosed herein) to be effective in treating a cancer can be assayed. In this method, a sample from the subject is assayed for the presence of elevated Lysosomal lipase A (LIPA) expression or increased activity of LIPA and when the sample indicates the presence of elevated LIPA or increased activity of LIPA, the subject is treatable, e.g., would get a therapeutic benefit, by a composition comprising the compound of Formula II:

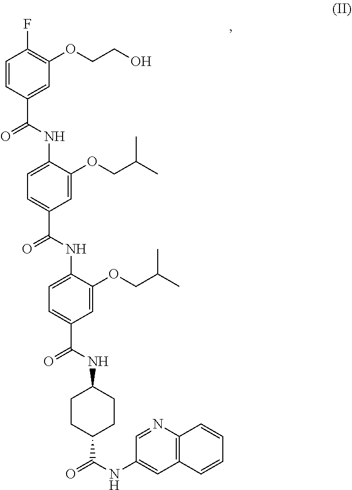 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

Elevated LIPA expression and increased activity of LIPA may be assayed from a sample from a subject. As examples, expression or activity may be detected by Western Blot or a similar immunoassay or by a nucleic acid amplification method including RT-PCR or real-time PCR or the like. It is also possible to detect changes in signalling partners down-stream of LIPA, including biochemical assays showing interactions between LIPA and its partners and/or phosphorylation of these players or changes in enzymatic activities by these players.

In various embodiments, the method further comprises administering a therapeutically-effective amount of the composition.

In some embodiments, the compound is a pharmaceutically acceptable salt of Formula II.

In several embodiments, the compound of Formula II inhibits LIPA activity when contacted to the LXXLL motif on LIPA.

In embodiments, the compound or composition is administered in an amount sufficient to induce endoplasmic reticulum stress, in an amount sufficient to inhibit activity of LIPA, and/or in an amount sufficient to reduce protein synthesis.

### Example 10

Triple negative breast cancer (TNBC), so called because these cancer cells are negative for the expression of the estrogen receptor (ERα or progesterone receptor (PR) or the HER2 protein, and accounts for approximately 15% of new breast cancer diagnoses.¹⁵ Women with TNBC are typically younger and more likely to be diagnosed with metastatic disease at presentation compared to the more common Ea-positive breast cancer. TNBCs are more aggressive tumors, grow and spread faster and have the highest mortality rate among all breast cancer subtypes: 150,000 deaths worldwide were attributed to metastatic TNBC in 2018 alone.^{16,17} Current therapies in TNBC are limited by the intertumoral and intratumoral heterogeneity. There is thus an urgent unmet need for effective targeted therapy options that target fundamental vulnerabilities in TNBC to overcome the issues with tumor heterogeneity.

The endoplasmic reticulum (ER) is a specialized organelle that participate in multiple cellular functions, especially in protein folding and maturation. Since TNBC cells have a high growth rate, they have a sustained and enhanced demand for de novo protein synthesis, folding and maturation. TNBC tumor growth, metastasis, chemotherapy, hostile environmental conditions, such as hypoxia, and oxidative stress further jeopardize the fidelity of protein folding in the ER, and cause ER stress, affecting maintenance of cellular proteostasis.¹⁸ To overcome the deleterious effects of ER stress, cells activate adaptive strategies such as UPR.¹⁹ PERK functions as a master sensor of ER stress and ameliorates ER stress through activation of eIF2α, decreasing de novo protein translation and thus reduce stress on ER protein processing.^{20,21} UPR can resolve ER stress and restore homeostasis: however unresolved ER stress can be lethal to cells via ER stress-induced apoptosis. UPR associated proteins 78-kDa glucose-regulated protein (GRP78), PERK, and Activating Transcription Factor 6 (ATF6) are overexpressed in TNBC, increase during TNBC progression and correlate with poor patient survival in TNBC.^{22,23} Several genome screens identified components of the ER stress pathway as targets of vulnerability in various cancers including TNBC.²⁴⁻²⁶

TNBC cells have a higher basal level of chronic and mild ER stress, which can help protect cancer cells by triggering UPR and helping them adapt to extreme conditions, such as hypoxia and nutrient deprivation. However, persistent and severe ER stress kills cancer cells by inducing their autophagy, apoptosis, necroptosis, or immunogenic cell death. Based on this rationale, several agents have been developed for triggering ER stress in cancer cells by targeting various processes in the secretory pathway, like tunicamycin, thapsigargin or bortezomib. In this disclosure, the inventors had a serendipitous discovery of oligobenzamides including compounds of Formula I, e.g., ERX-41, with activity against TNBC. The inventors' goal in lead optimization of ERX-11 was to develop a more potent agent targeting breast cancer. The finding of ERX-41 as a hit in TNBC confirms the inventors' previous observations that even minor changes in oligobenzamide can significantly alter their specificity and activity. These findings are robust and indicate that ERX-41 has activity against multiple molecular subtypes of TNBC, across >20 distinct TNBC cell lines, >10 patient derived tumor models and 7 distinct xenograft models. Patient derived explant data are particularly important as they represent the patients who would be eligible for treatment with ERX-41.

The inventors have shown that ERX-41 induces ER stress, using a variety of biochemical and ultrastructural studies. The inventors' confocal microscopy images showing ER stress are noteworthy, since ER luminal diameter (50-60 nm) normally is below the resolution of confocal microscopy (100-200 nm). Since the measured ER dilation following ERX-41 treatment approaches 600 nm, the true fold effect of ERX-41 on ER morphology is profound and significant. In addition, these studies clearly support that LIPA is central to ERX-41 ability to induce ER stress, as evidenced by the inventors' biochemical and ultrastructural studies confirming the effect of knockdown of LIPA in TNBC cells. The inventors observed that ERX-41 induces ER stress, shuts down de novo protein synthesis, blocks the proliferation, and induces apoptosis of TNBC cells in vitro, ex vivo and in vivo. These results suggest that ERX-41 aggravates this already engaged system in TNBC to exhaust its protective features and cause apoptosis. In normal cells and tissues, ERX-41 does not induce ER stress, suggesting that the basal level of ER stress and the compensatory UPR pathway may dictate responsiveness to ERX-41. These data indicate that ERX-41 targets a fundamental vulnerability in TNBC—the high basal level of ER stress and may be able to overcome the intertumoral and intratumoral heterogeneity of TNBC.

The ER is also a major site of lipid metabolism and many enzymes involved in lipid metabolism are located in the ER.²⁷ Although the UPR was implicated in protein homeostasis in the ER, it also plays essential roles in maintaining lipid homeostasis. For example several components of UPR axis such as ATF4²⁸ IRE1α/XBP1²⁹, ATF6³⁰ and CHOP³¹ are involved in the lipid metabolism in ER. Further, UPR regulates the quantity of ER driven by synthesis of lipids.³² Due to extensive cross talk of UPR axis with lipid metabolism, drugs targeting lipid homeostasis may have pathological implications in UPR response. In the liver, the LAL protein is a key regulator of cellular lipid homeostasis as a consequence of its lipase function.

Indeed, LAL protein play an important role in breaking down lipids such as triglycerides and cholesteryl esters. Clinical and biochemical features of LAL-deficiency are a consequence of accumulation of cholesteryl esters in the lysosomes. Since the catalytic activity of LAL is not targeted by ERX-41, the inventors have noted no significant changes in lysosomes following ERX-41 treatment. Further, Lalistat 2, a known inhibitor of LAL lipase activity, does not induce ER stress or cell death in TNBC. These studies suggest that LAL localization to the ER is critical for ERX-41 activity: indeed, a recombinant LAL that lacks the signal peptide does not get glycosylated or respond to ERX-41.

The inventors' unbiased proteomic studies offer some insight as to how ERX-41 binding to LAL causes ER stress. The have identified the interactome of LAL, which appears to be primarily composed of ER-resident proteins and proteins involved in critical ER maturation functions such as ER folding. Importantly, the inventors' unbiased global proteomic studies indicate that ERX-41 causes a decrease in the expression of a number of proteins, including several known ER-resident proteins and proteins involved in ER folding. The inventors have validated that ERX-41 binding to LAL causes these proteins to be down-regulated in an LIPA-dependent manner. These data suggest that ERX-41 binding to LAL causes a down-regulation of several ER proteins involved in protein maturation, and effectively cause ER stress.

From an unbiased CRISPR KO screen, these studies have identified LIPA as the cellular target of ERX-41 in TNBC. the inventors' target identification was validated in multiple TNBC cell lines and confirmed that KO of LIPA abrogates responsiveness to ERX-41. Since the oligobenzamides were derived from ERX-11 which was designed to target a LXXLL motif, it is not surprising that ERX-41 binds to the LXXLL motif of LIPA. Deletion of the LXXLL motif or point mutation(s) within the LXXLL motif blocks LAL binding to ERX-41 and the ability of ERX-41 to induce ER stress and cell death. While the LXXLL motif was initially identified as a nuclear receptor box, multiple protein-protein interactions occur through the LXXLL motif, which typically adopts a helical secondary structure. The inventors and others have previously shown that the sequences flanking the LXXLL motif determine specificity of interactions: these studies indicate that binding of ERX-41 to LAL may involve some residues in the LXXLL domain. Importantly, unlike ERX-11, ERX-41 does not bind ERα: similarly, while ERX-41 but not ERX-11 bind LAL. The identification of a single point mutation within LIPA that abrogates responsiveness to ERX-41 represents gold standard validation that LAL is the target of ERX-41. Further mutational LIPA studies and co-crystallization are ongoing and will enable detailed characterization of the ERX-41 binding site.

Knockout of LIPA abrogates the ability of ERX-41 to induce ER stress, UPR, block de novo protein synthesis and cause cell death in vitro and in vivo. Reconstitution of LIPA in this knockout system restores the ability of ERX-41 to induce ER stress, UPR and cause cell death. Importantly, reconstitution of a LIPA mutant in this knockout system that is incapable of binding ERX-41, does not restore the ability of ERX-41 to induce ER stress, UPR and cause cell death. These data establish that the interaction between ERX-41 and LAL is critical for ERX-41 induced cell death. The inventors also have shown that LAL binds to ERX-41 using two distinct assays—the cellular thermal shift assay in intact cells and biotinylated pulldowns of LAL from cell lysates. Co-crystallization of LAL with ERX-41 will further inform structural details of the interaction and are ongoing. Collectively, these data confirm the central role of LIPA in the activity of ERX-41 against TNBC.

The identification of LAL as a targetable molecular vulnerability is a critical finding in this disclosure. The inventors' chemistry-first approach enabled the identification of a novel function of LAL related to its ability to function as a molecular chaperone of ER-resident proteins involved in protein folding and to cause ER stress. While LAL protein levels in tumors have been inadequately profiled, the expression of LIPA mRNA appears to be highest in breast, ovarian, glioma and pancreatic cancers (Human protein atlas). These studies indicate that cell lines representing these tumor types express LAL and are sensitive to ERX-41 treatment both in vitro and in vivo. LIPA knockdown in these cell lines abrogates responsiveness to ERX-41, confirming that LAL is the molecular target of ERX-41. Further studies are needed to evaluate if the LAL expression levels and basal level of ER stress in tumors could serve as a biomarker of cellular response to ERX-41. These data clearly support the development of additional LAL targeting agents and for further lead optimization of ERX-41.

This disclosure reveals the important finding of a potent therapeutic agent (ERX-41) with a novel target (LAL) and mechanism of action (disruption of protein folding and induction of ER stress) that may have utility in treating TNBC. Ongoing studies will define more potent analogs of ERX-41 based on crystallographic characterization and further mechanistic insights of how ERX-41 binding to LAL disrupts the expression of ER resident proteins involved in protein folding and causing ER stress in TNBC. These studies will also pave the path forward to lead optimization and clinical translation of ERX-41 and offer hope for patients with TNBC and possibly other cancers.

Finally, the data presented herein suggest that other compounds of Formula I, i.e., the compound of Formula II (ERX-315), would likewise provide a therapeutic benefit to human subjects having cancer, e.g., TNBC.

### Example 11

Experiments as described in Examples 1 and 3 were repeated using the compound of Formula IV (TK-342). Shown in FIGS. 19A-B are results of MTT assays demonstrating the effect of TK-342 (Formula IV) on cell viability of SUM-159 cells (WT) and SUM-159 cells with LIPA KO and LIPA OE, as well as cell viability of SUM-159 cells (WT) and SUM-159 cells with SOAT1. These data suggest that TK-342 would provide a therapeutic benefit to human subjects having cancer, e.g., TNBC and/or a LIPA-related cancer.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Anderson, Practical Process Research & Development—A Guide for Organic Chemists, 2^{nd} ed., Academic Press, New York, 2012.
Handbook of Pharmaceutical Salts: Properties, and Use, Stahl and Wermuth Eds., Verlag Helvetica Chimica Acta, 2002.
Reagan-Shaw et al., FASEB J., 22(3):659-661, 2008.
Smith, March's Advanced Organic Chemistry: Reactions, Mechanisms, and Structure, 7^{th} Ed., Wiley, 2013.
U.S. Patent Pub. 2009/0012141
WO2020117715A1
Ahn et al., Mini-Rev. Med. Chem., 2:463-473, 2002.
Marshall, Tetrahedron, 49:3547-3558, 1993.
1 McInerney, E. M. et al. Determinants of coactivator LXXLL motif specificity in nuclear receptor transcriptional activation. Genes Dev 12, 3357-3368 (1998).
2 Heery, D. M., Kalkhoven, E., Hoare, S. & Parker, M. G. A signature motif in transcriptional co-activators mediates binding to nuclear receptors. Nature 387, 733-736, doi:10.1038/42750 (1997).
3 Plevin, M. J., Mills, M. M. & Ikura, M. The LxxLL motif: a multifunctional binding sequence in transcriptional regulation. Trends Biochem Sci 30, 66-69, doi:10.1016/j.tibs.2004.12.001 (2005).
4 McInerney, E. M. et al. Determinants of coactivator LXXLL motif specificity in nuclear receptor transcriptional activation. Genes Dev 12, 3357-3368, doi:10.1101/gad.12.21.3357 (1998).
5 Chang, C. et al. Dissection of the LXXLL nuclear receptor-coactivator interaction motif using combinatorial peptide libraries: discovery of peptide antagonists of estrogen receptors alpha and beta. Mol Cell Biol 19, 8226-8239, doi:10.1128/mcb.19.12.8226 (1999).
6 Ravindranathan, P. et al. Peptidomimetic targeting of critical androgen receptor-coregulator interactions in prostate cancer. Nat Commun 4, 1923, doi:10.1038/ncomms2912 (2013).
7 Raj, G. V. et al. Estrogen receptor coregulator binding modulators (ERXs) effectively target estrogen receptor positive human breast cancers. Elife 6, doi:10.7554/eLife.26857 (2017).
8 Strom, T. B. et al. Lysosomal acid lipase does not have a propeptide and should not be considered being a proprotein. Proteins 88, 440-448, doi:10.1002/prot.25821 (2020).
9 Dean, J. L. et al. Therapeutic response to CDK4/6 inhibition in breast cancer defined by ex vivo analyses of human tumors. Cell Cycle 11, 2756-2761, doi:10.4161/cc.21195 (2012).
10 Mohammed, H. et al. Progesterone receptor modulates ERalpha action in breast cancer. Nature 523, 313-317, doi:10.1038/nature14583 (2015).
11 Regan Anderson, T. M. et al. Breast Tumor Kinase (Brk/PTK6) Is Induced by HIF, Glucocorticoid Receptor, and PELP1-Mediated Stress Signaling in Triple-Negative Breast Cancer. Cancer Res 76, 1653-1663, doi:10.1158/0008-5472.CAN-15-2510 (2016).
12 Li, F. & Zhang, H. Lysosomal Acid Lipase in Lipid Metabolism and Beyond. Arterioscler Thromb Vasc Biol 39, 850-856, doi:10.1161/ATVBAHA.119.312136 (2019).
13 Rosenbaum, A. I. et al. Chemical screen to reduce sterol accumulation in Niemann-Pick C disease cells identifies novel lysosomal acid lipase inhibitors. Biochim Biophys Acta 1791, 1155-1165, doi:10.1016/j.bbalip.2009.08.005 (2009).
14 Rosenbaum, A. I. et al. Thiadiazole carbamates: potent inhibitors of lysosomal acid lipase and potential Niemann-Pick type C disease therapeutics. J Med Chem 53, 5281-5289, doi:10.1021/jm100499s (2010).
15 Foulkes, W. D., Smith, I. E. & Reis-Filho, J. S. Triple-negative breast cancer. N. Engl. J. Med 363, 1938-1948 (2010).
16 Carey, L., Winer, E., Viale, G., Cameron, D. & Gianni, L. Triple-negative breast cancer: disease entity or title of convenience? Nat. Rev. Clin. Oncol 7, 683-692 (2010).
17 Comprehensive molecular portraits of human breast tumours. Nature 490, 61-70 (2012).
18 Wang, M. & Kaufman, R. J. The impact of the endoplasmic reticulum protein-folding environment on cancer development. Nat Rev Cancer 14, 581-597, doi:10.1038/nrc3800 (2014).
19 Schonthal, A. H. Targeting endoplasmic reticulum stress for cancer therapy. Front Biosci (Schol Ed) 4, 412-431, doi:10.2741/276 (2012).
20 Schroder, M. & Kaufman, R. J. The mammalian unfolded protein response. Annu Rev Biochem 74, 739-789, doi:10.1146/annurev.biochem.73.011303.074134 (2005).
21 Almanza, A. et al. Endoplasmic reticulum stress signalling—from basic mechanisms to clinical applications. FEBS J286, 241-278, doi:10.1111/febs.14608 (2019).
22 McGrath, E. P. et al. The Unfolded Protein Response in Breast Cancer. Cancers (Basel) 10, doi:10.3390/cancers10100344 (2018).
23 Yang, C. et al. Expression of glucose-regulated protein 78 as prognostic biomarkers for triple-negative breast cancer. Histol Histopathol 35, 559-568, doi:10.14670/HH-18-185 (2020).
24 Nijhawan, D. et al. Cancer vulnerabilities unveiled by genomic loss. Cell 150, 842-854, doi:10.1016/j.cell.2012.07.023 (2012).
25 Cheung, H. W. et al. Systematic investigation of genetic vulnerabilities across cancer cell lines reveals lineage-specific dependencies in ovarian cancer. Proc Nat Acad Sci USA 108, 12372-12377, doi:10.1073/pnas.1109363108 (2011).
26 Marcotte, R. et al. Essential gene profiles in breast, pancreatic, and ovarian cancer cells. Cancer Discov 2, 172-189, doi:10.1158/2159-8290.CD-11-0224 (2012).
27 Han, J. & Kaufman, R. J. The role of ER stress in lipid metabolism and lipotoxicity. J Lipid Res 57, 1329-1338, doi:10.1194/jlr.R067595 (2016).
28 Li, H. et al. ATF4 deficiency protects mice from high-carbohydrate-diet-induced liver steatosis. Biochem J 438, 283-289, doi:10.1042/BJ20110263 (2011).
29 Zhang, K. et al. The unfolded protein response transducer IRE1alpha prevents ER stress-induced hepatic steatosis. EMBO J 30, 1357-1375, doi:10.1038/emboj.2011.52 (2011).
30 Zeng, L. et al. ATF6 modulates SREBP2-mediated lipogenesis. EMBO J 23, 950-958, doi:10.1038/sj.emboj.7600106 (2004).
31 Rutkowski, D. T. et al. UPR pathways combine to prevent hepatic steatosis caused by ER stress-mediated suppression of transcriptional master regulators. Dev Cell 15, 829-840, doi:10.1016/j.devcel.2008.10.015 (2008).
32 Schuck, S., Prinz, W. A., Thom, K. S., Voss, C. & Walter, P. Membrane expansion alleviates endoplasmic reticulum stress independently of the unfolded protein response. J Cell Biol 187, 525-536, doi:10.1083/jcb.200907074 (2009).
33 Viswanadhapalli, S. et al. EC359: A First-in-Class Small-Molecule Inhibitor for Targeting Oncogenic LIFR Signaling in Triple-Negative Breast Cancer. Mol Cancer Ther 18, 1341-1354, doi:10.1158/1535-7163.MCT-18-1258 (2019).
34 Shalem, O. et al. Genome-scale CRISPR-Cas9 knockout screening in human cells. Science 343, 84-87, doi:10.1126/science.1247005 (2014).
35 Liang, J. R., Lingeman, E., Ahmed, S. & Corn, J. E. Atlastins remodel the endoplasmic reticulum for selective autophagy. J Cell Biol 217, 3354-3367, doi:10.1083/jcb.201804185 (2018).
36 Jafari, R. et aL. The cellular thermal shift assay for evaluating drug target interactions in cells. Nat Protoc 9, 2100-2122, doi:10.1038/nprot.2014.138 (2014).
37 Dairaku, T. et al. A practical fluorometric assay method to measure lysosomal acid lipase activity in dried blood spots for the screening of cholesteryl ester storage disease and Wolman disease. Mol Genet Metab 111, 193-196, doi:10.1016/j.ymgme.2013.11.003 (2014).
38 Viswanadhapalli, S. et aL. EC359-A first-in-class small molecule inhibitor for targeting oncogenic LIFR signaling in triple negative breast cancer. Mol Cancer Ther, doi:10.1158/1535-7163.MCT-18-1258 (2019).
39 Mann, M., Cortez, V. & Vadlamudi, R. PELP1 oncogenic functions involve CARM1 regulation. Carcinogenesis 34, 1468-1475, doi:10.1093/carcin/bgt091 (2013).
40 Searle, B. C. et al. Chromatogram libraries improve peptide detection and quantification by data independent acquisition mass spectrometry. Nat Commun 9, 5128, doi:10.1038/s41467-018-07454-w (2018).
41 Rosenberger, G. et al. A repository of assays to quantify 10,000 human proteins by SWATH-MS. Sci Data 1, 140031, doi:10.1038/sdata.2014.31 (2014).

1. A method for treating a cancer characterized by having increased activity of Lysosomal lipase A (LIPA) comprising administering a therapeutically-effective amount of a composition comprising a compound of Formula I: 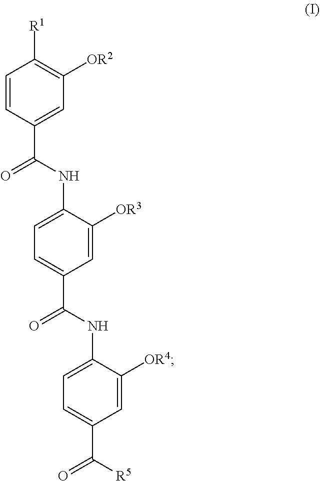
to a subject having a cancer with elevated LIPA expression.

2. A method for inhibiting activity of Lysosomal lipase A (LIPA) in a subject having a cancer comprising administering a therapeutically-effective amount of a composition comprising a compound of Formula L: 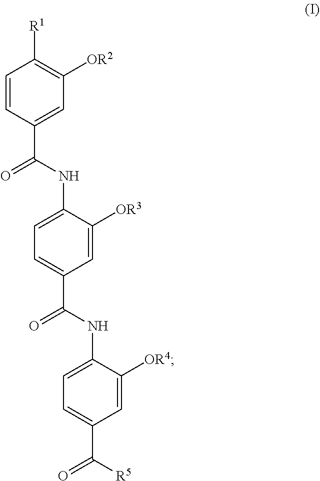
to the subject.

3. A method for treating cancer in a subject in need thereof comprising testing a sample from the subject for the presence of elevated Lysosomal lipase A (LIPA) expression and administering a therapeutically-effective amount of a composition comprising a compound of Formula I: 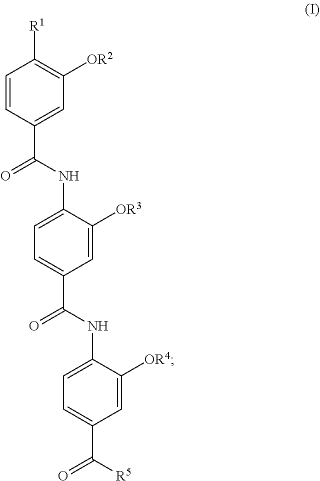
to a subject that provided a sample identified as having an elevated LIPA expression.

4. The method of any one of claims 1 to 3, wherein the compound of Formula I inhibits LIPA activity when contacted to the LXXLL motif on LIPA.

5. The method of any one of claims 1 to 4, wherein the compound is a pharmaceutically acceptable salt of Formula I.

6. The method of any one of claims 1 to 5, wherein:
R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein:
R^{1a} is aralkyl_{(C<18)}, substituted aralkyl_{(C<18)}, or the side chain of a canonical amino acid;
R², R³, and R⁴ are each independently alkyl_{(C<12)}, substituted alkyl_{(C<12)}, aralkyl_{(C<18)}, or substituted aralkyl_{(C<18)}; and
R⁵ is —OR^{5a} or —NHR^{5b}, wherein:
R^{5a} is alkyl_{(C<12)} or substituted alkyl_{(C<12)};
R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or
a group of the formula: 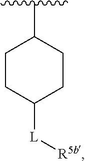
wherein;
L is —CO₂— or —C(O)NR^{L}—, wherein:
R^{L} hydrogen, alkyl_{(C<12)}, or substituted alkyl_{(C<12)};
R^{5b′} is aryl_{(C<12)}, aralkyl_{(C<18)}, heteroaryl_{(c<12)}, heteroaralkyl_{(C<18)},
or a substituted version of any of these groups.

7. The method of any one of claims 1 to 6, wherein the composition comprises the compound of Formula II:

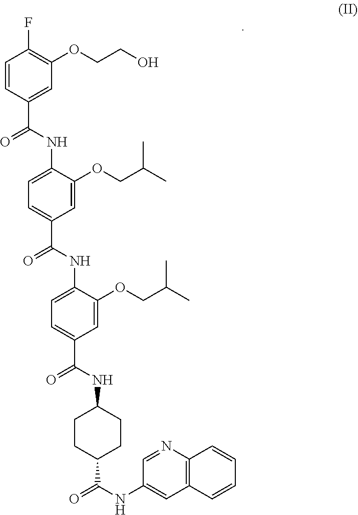 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

8. The method of any one of claims 1 to 6, wherein the composition comprises the compound of Formula III:

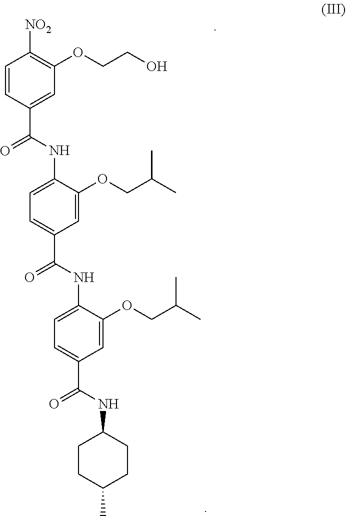 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

9. The method of any one of claims 1 to 6, wherein the composition comprises the compound of Formula IV:

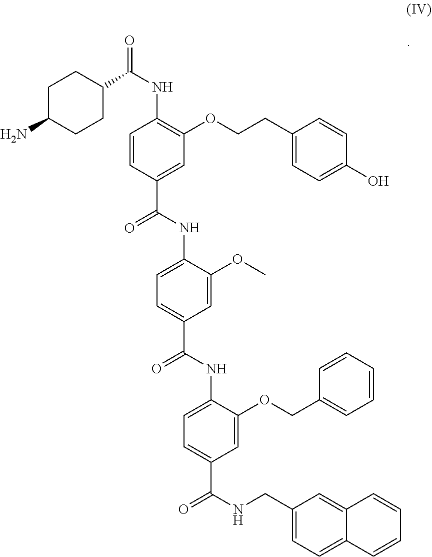

10. The method ofany one of claims 1 to 9, wherein the subject is a mammal.

11. The method of any one of claims 1 to 10, wherein the subject is a human.

12. The method of any one of claims 1 to 11, wherein the cancer is a therapy-resistant cancer.

13. The method of any one of claims 1 to 12, wherein the cancer is breast cancer, ovarian cancer, pancreatic cancer, or brain cancer.

14. The method of any one of claims 1 to 13, wherein the cancer is breast cancer.

15. The method of claim 14, wherein the breast cancer is triple negative breast cancer.

16. The method of claim 14, wherein the breast cancer is an estrogen receptor-positive cancer.

17. The method of claim 14, wherein the breast cancer is an estrogen receptor-negative cancer.

18. The method of claim 13, wherein the cancer is ovarian cancer.

19. The method of claim 13, wherein the cancer is pancreatic cancer.

20. The method of claim 13, wherein the cancer is brain cancer.

21. The method of claim 20, wherein the brain cancer is glioblastoma.

22. The method of any one of claims 1 to 21, wherein administering comprises intravenous, intra-arterial, intra-tumoral, subcutaneous, topical, or intraperitoneal administration.

23. The method of any one of claims 1 to 22, wherein administering comprises local, regional, systemic, or continual administration.

24. The method of any one of claims 1 to 23, further comprising providing to said subject a second anti-cancer therapy.

25. The method of claim 24, wherein said second anti-cancer therapy is surgery, chemotherapy, radiotherapy, hormonal therapy, toxin therapy, immunotherapy, and cryotherapy.

26. The method of claim 24 or claim 25, wherein said second anti-cancer therapy is provided prior to administering said composition.

27. The method of any one of claims 24 to 26, wherein said second anti-cancer therapy is provided after administering said composition.

28. The method of any one of claims 24 to 27, wherein said second anti-cancer therapy is provided contemporaneous with said composition.

29. The method of any one of claims 1 to 28, wherein said composition is administered daily.

30. The method of claim 29, wherein said composition is administered daily for 7 days, 2 weeks, 3 weeks, 4 weeks, one month, 6 weeks, 8 weeks, two months, 12 weeks, or 3 months.

31. The method of any one of claims 1 to 28, wherein said composition is administered weekly.

32. The method of claim 31, wherein said composition is administered weekly for 2 weeks, 3 weeks, 4 weeks, 6 weeks, 8 weeks, 10 weeks, or 12 weeks.

33. The method of any one of claims 1 to 32, wherein the compound or composition is administered in an amount sufficient to induce endoplasmic reticulum stress.

34. The method of any one of claims 1 to 33, wherein the compound or composition is administered in an amount sufficient to inhibit activity of LIPA.

35. The method of any one of claims 1 to 34, wherein the compound or composition is administered in an amount sufficient to reduce protein synthesis.

36. A composition for use in the method of any one of claims 1 to 35.

37. A composition for use in a method of treating a cancer characterized by having increased activity of Lysosomal lipase A (LIPA), the method comprising administering a therapeutically-effective amount of a composition comprising a compound of Formula I: 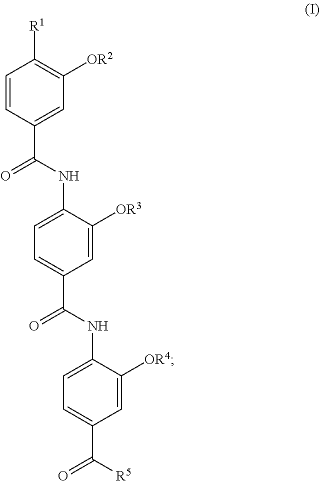
to a subject having a cancer with elevated LIPA expression.

38. A composition for use in a method of inhibiting activity of Lysosomal lipase A (LIPA) in a subject having a cancer, the method comprising administering a therapeutically-effective amount of a composition comprising a compound of Formula I: 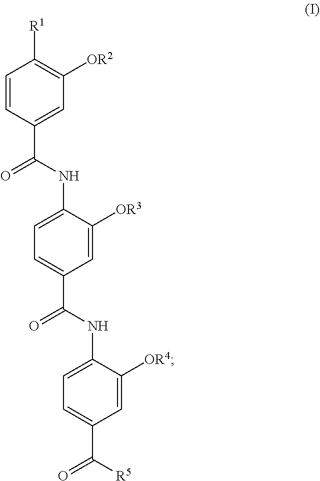
to the subject.

39. A composition for use in a method of treating cancer in a subject in need thereof, the method comprising testing a sample from the subject for the presence of elevated Lysosomal lipase A (LIPA) expression and administering a therapeutically-effective amount of a composition comprising a compound of Formula I: 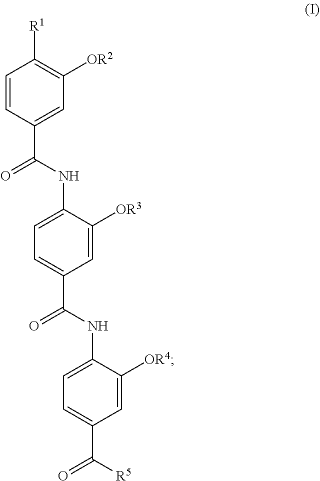
to a subject that provided a sample identified as having an elevated LIPA expression.

40. The composition of any one of claims 37 to 39, wherein the compound of Formula I inhibits LIPA activity when contacted to the LXXLL motif on LIPA.

41. The composition of any one of claims 37 to 40, wherein the compound is a pharmaceutically acceptable salt of Formula I.

42. The composition of any one of claims 37 to 41, wherein:
R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein:
R^{1a} is aralkyl_{(c<18)}, substituted aralkyl_{(c<18)}, or the side chain of a canonical amino acid;
R², R³, and R⁴ are each independently alkyl_{(C<12)}, substituted alkyl_{(C<12)}, aralkyl_{(C<18)}, or substituted aralkyl_{(C<18)}; and
R⁵ is —OR^{5a} or —NHR^{5b}, wherein:
R^{5a} is alkyl_{(C<12)} or substituted alkyl_{(C<12)};
R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or
a group of the formula: 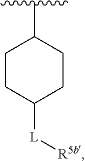
wherein;
L is —CO₂— or —C(O)NR^{L}—, wherein:
R^{L} hydrogen, alkyl_{(C<12)}, or substituted alkyl_{(C<12)};
R^{5b′} is aryl_{(C<12)}, aralkyl_{(C<18)}, heteroaryl_{(C<12)}, heteroaralkyl_{(C<18)},
or a substituted version of any of these groups.

43. The composition of any one of claims 37 to 42, wherein the composition comprises the compound of Formula II:

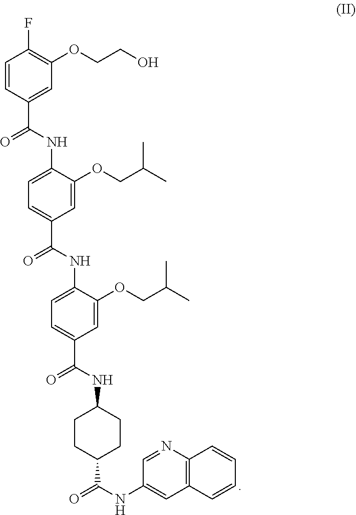 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

44. The composition of any one of claims 37 to 42, wherein the composition comprises the compound of Formula III:

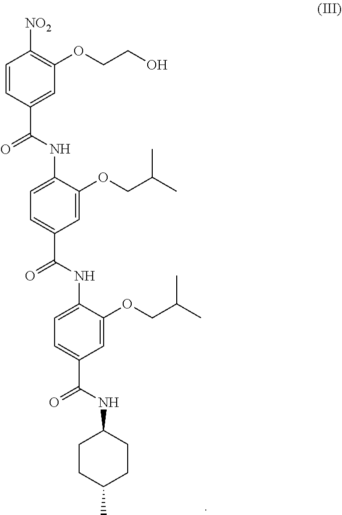 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

45. The composition of any one of claims 37 to 42, wherein the composition comprises the compound of Formula IV:

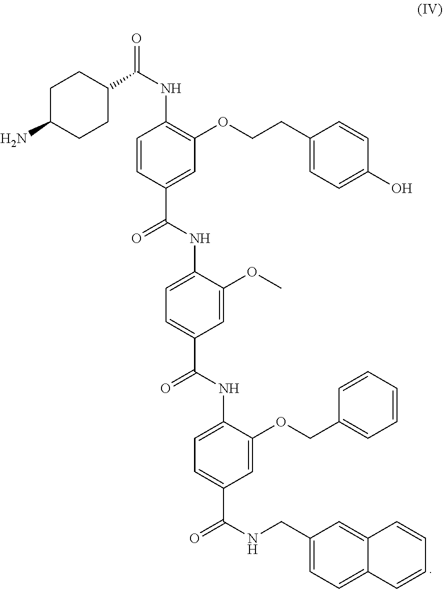

46. The composition of any one of claims 37 to 45, wherein the amount of the compound in the composition is sufficient to induce endoplasmic reticulum stress.

47. The composition of any one of claims 37 to 46, wherein the amount of the compound in the composition is sufficient to inhibit activity of LIPA.

48. The composition of any one of claims 37 to 47, wherein the amount of the compound in the composition is sufficient to reduce protein synthesis.

49. The composition of any one of claims 37 to 48, wherein the cancer is breast cancer, ovarian cancer, pancreatic cancer, or brain cancer.

50. The composition of any one of claims 37 to 49, wherein the cancer is breast cancer.

51. The composition of claim 50, wherein the breast cancer is triple negative breast cancer.

52. The composition of claim 50, wherein the breast cancer is an estrogen receptor-positive cancer.

53. The composition of claim 50, wherein the breast cancer is an estrogen receptor-negative cancer.

54. A method for determining if a subject would be treated by a composition comprising a compound of Formula I: 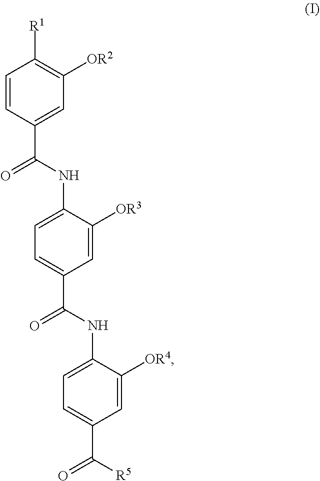
the method comprising testing a sample from the subject for the presence of elevated Lysosomal lipase A (LIPA) expression and when the sample indicates the presence of elevated LIPA or increased activity of LIPA, the subject is treatable by a composition comprising a compound of Formula I.

55. The method of claim 54, wherein the subject has a cancer.

56. The method of claim 55, wherein the cancer is breast cancer, ovarian cancer, pancreatic cancer, or brain cancer.

57. The method of any one of claims 54 to 56, wherein the compound is a pharmaceutically acceptable salt of Formula I.

58. The method of any one of claims 54 to 57, wherein:
R¹ is halogen, —NO₂, alkyl_{(C<12)}, substituted alkyl_{(C<12)}, amido_{(C<12)}, substituted amido_{(C<12)}, or —NHC(O)CH(R^{1a})NH₂, wherein:
R^{1a} is aralkyl_{(C<18)}, substituted aralkyl_{(C<18)}, or the side chain of a canonical amino acid;
R², R³, and R⁴ are each independently alkyl_{(C<12)}, substituted alkyl_{(C<12)}, aralkyl_{(C<18)}, or substituted aralkyl_{(C<18)}; and
R⁵ is —OR^{5a} or —NHR^{5b}, wherein:
R^{5a} is alkyl_{(C<12)} or substituted alkyl_{(C<12)};
R^{5b} is cycloalkyl_{(C<12)}, aryl_{(C<12)}, aralkyl_{(C<12)}, heteroaryl_{(C<12)}, heteroaralkyl_{(C<18)}, or a substituted version of any of these groups; or
a group of the formula: 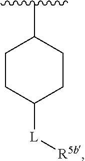
wherein;
L is —CO₂— or —C(O)NR^{L}—, wherein:
R^{L} hydrogen, alkyl_{(C<12)}, or substituted alkyl_{(C<12)};
R^{5b′} is aryl_{(C<12)}, aralkyl_{(C<18)}, heteroaryl_{(C<12)}, heteroaralkyl_{(C<18)},
or a substituted version of any of these groups.

59. The method of any one of claims 54 to 58, wherein the compound comprises Formula II:

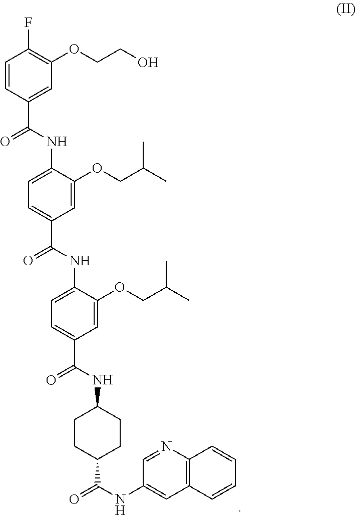 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]

60. The method of any one of claims 54 to 58, wherein the compound comprises Formula III:

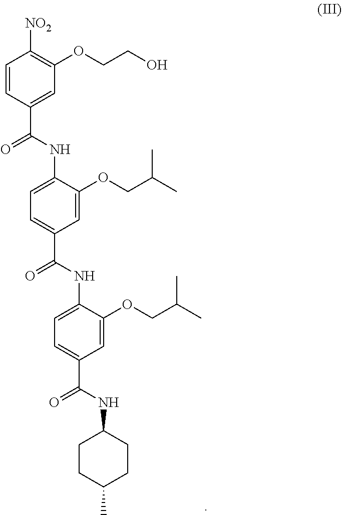 [SMILES: CC(C)COc1cc(C(=O)Nc2ccc(C(=O)N[C@H]3CC[C@H](C)CC3)cc2OCC(C)C)ccc1NC(=O)c1ccc([N+](=O)[O-])c(OCCO)c1]

61. The method of any one of claims 54 to 58, wherein the compound comprises Formula IV:

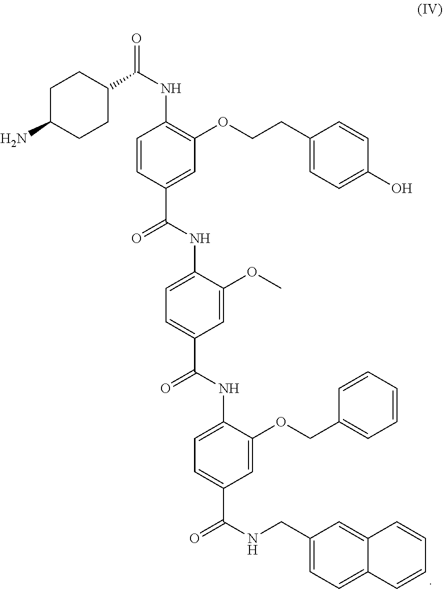

62. The method of any one of claims 54 to 61, wherein the method further comprises administering a therapeutically-effective amount of the composition.

63. The method of claim 62, wherein the amount of the compound in the composition is sufficient to induce endoplasmic reticulum stress.

64. The method of claim 62 or claim 63, wherein, wherein the amount of the compound in the composition is sufficient to inhibit activity of LIPA.

65. The method of any one of claims 62 to 64, wherein the amount of the compound in the composition is sufficient to shut down protein synthesis.

66. A method for treating a cancer characterized by having increased activity of Lysosomal lipase A (LIPA) comprising administering a therapeutically-effective amount of a composition comprising the compound of Formula II: 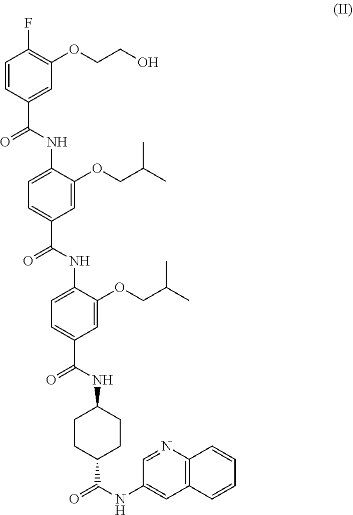 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]
to a subject having a cancer with elevated LIPA expression.

67. A method for inhibiting activity of Lysosomal lipase A (LIPA) in a subject having a cancer comprising administering a therapeutically-effective amount of a composition comprising the compound of Formula II: 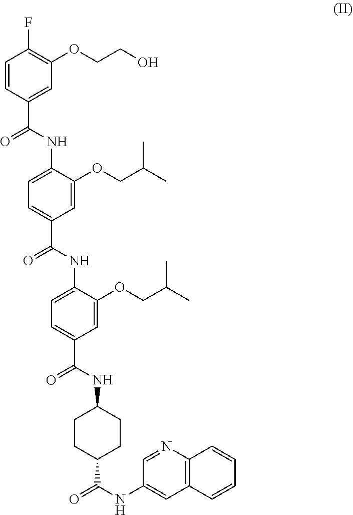 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]
to the subject.

68. A method for treating cancer in a subject in need thereof comprising testing a sample from the subject for the presence of elevated Lysosomal lipase A (LIPA) expression and administering a therapeutically-effective amount of a composition comprising the compound of Formula II: 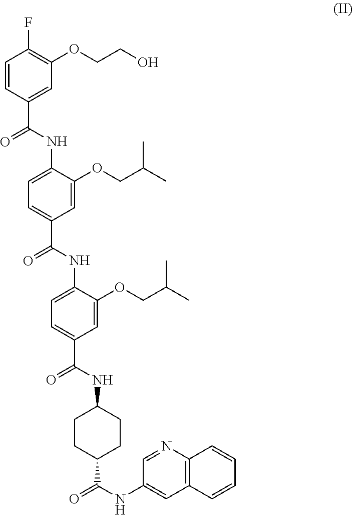 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]
to a subject that provided a sample identified as having an elevated LIPA expression.

69. The method of any one of claims 66 to 68, wherein the subject is a mammal.

70. The method of any one of claims 66 to 69 wherein the subject is a human.

71. The method of any one of claims 66 to 70, wherein the cancer is a therapy-resistant cancer.

72. The method of any one of claims 66 to 71, wherein the cancer is breast cancer, ovarian cancer, pancreatic cancer, or brain cancer.

73. The method of any one of claims 66 to 72, wherein the cancer is breast cancer.

74. The method of claim 73, wherein the breast cancer is triple negative breast cancer.

75. The method of claim 73, wherein the breast cancer is an estrogen receptor-positive cancer.

76. The method of claim 73, wherein the breast cancer is an estrogen receptor-negative cancer.

77. The method of claim 72, wherein the cancer is ovarian cancer.

78. The method of claim 72, wherein the cancer is pancreatic cancer.

79. The method of claim 72, wherein the cancer is brain cancer.

80. The method of claim 79, wherein the brain cancer is glioblastoma.

81. The method of any one of claims 66 to 80, wherein administering comprises intravenous, intra-arterial, intra-tumoral, subcutaneous, topical, or intraperitoneal administration.

82. The method of any one of claims 66 to 81, wherein administering comprises local, regional, systemic, or continual administration.

83. The method of any one of claims 66 to 82, further comprising providing to said subject a second anti-cancer therapy.

84. The method of claim 83, wherein said second anti-cancer therapy is surgery, chemotherapy, radiotherapy, hormonal therapy, toxin therapy, immunotherapy, and cryotherapy.

85. The method of claim 83 or claim 84, wherein said second anti-cancer therapy is provided prior to administering said composition.

86. The method of any one of claims 83 to 85, wherein said second anti-cancer therapy is provided after administering said composition.

87. The method of any one of claims 83 to 86, wherein said second anti-cancer therapy is provided contemporaneous with said composition.

88. The method of any one of claims 66 to 87, wherein said composition is administered daily.

89. The method of claim 88, wherein said composition is administered daily for 7 days, 2 weeks, 3 weeks, 4 weeks, one month, 6 weeks, 8 weeks, two months, 12 weeks, or 3 months.

90. The method of any one of claims 66 to 87, wherein said composition is administered weekly.

91. The method of claim 90, wherein said composition is administered weekly for 2 weeks, 3 weeks, 4 weeks, 6 weeks, 8 weeks, 10 weeks, or 12 weeks.

92. The method of any one of claims 66 to 91, wherein the compound or composition is administered in an amount sufficient to induce endoplasmic reticulum stress.

93. The method of any one of claims 66 to 92, wherein the compound or composition is administered in an amount sufficient to inhibit activity of LIPA.

94. The method of any one of claims 66 to 93, wherein the compound or composition is administered in an amount sufficient to reduce protein synthesis.

95. A composition for use in a method of treating a cancer characterized by having increased activity of Lysosomal lipase A (LIPA), the method comprising administering a therapeutically-effective amount of a composition comprising the compound of Formula II: 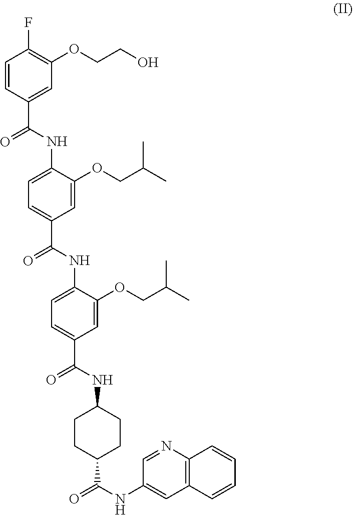 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]
to a subject having a cancer with elevated LIPA expression.

96. A composition for use in a method of inhibiting activity of Lysosomal lipase A (LIPA) in a subject having a cancer, the method comprising administering a therapeutically-effective amount of a composition comprising the compound of Formula II: 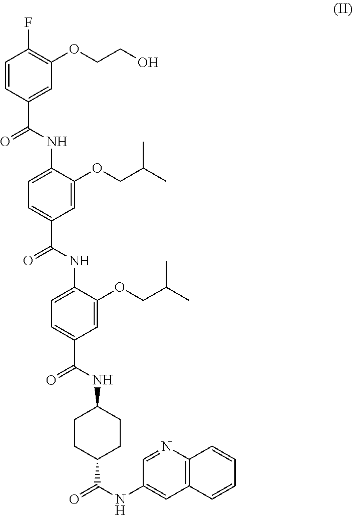 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]
to the subject.

97. A composition for use in a method of treating cancer in a subject in need thereof, the method comprising testing a sample from the subject for the presence of elevated Lysosomal lipase A (LIPA) expression and administering a therapeutically-effective amount of a composition comprising the compound of Formula II: 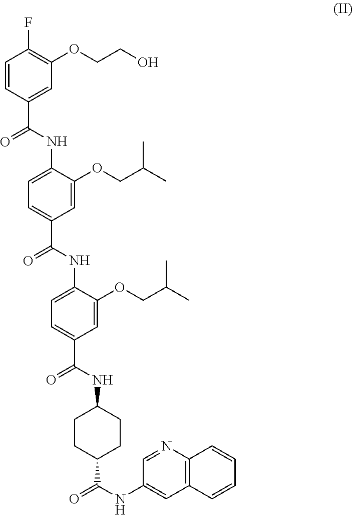 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]
to a subject that provided a sample identified as having an elevated LIPA expression.

98. The composition of any one of claims 95 to 97, wherein the amount of the compound in the composition is sufficient to induce endoplasmic reticulum stress.

99. The composition of any one of claims 95 to 98, wherein, wherein the amount of the compound in the composition is sufficient to inhibit activity of LIPA.

100. The composition of any one of claims 95 to 99, wherein the amount of the compound in the composition is sufficient to shut down protein synthesis.

101. A method for determining if a subject would be treated by a composition comprising the compound of Formula II: 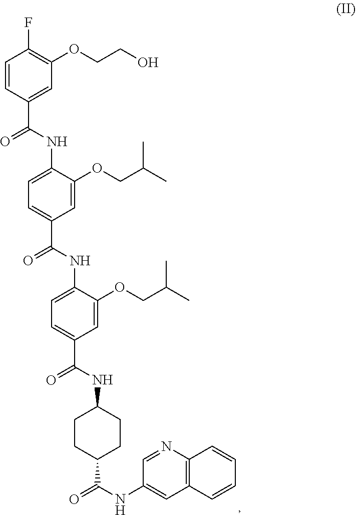 [SMILES: CC(C)COc1cc(C(=O)Nc2ccc(C(=O)N[C@H]3CC[C@H](C(=O)Nc4cnc5ccccc5c4)CC3)cc2OCC(C)C)ccc1NC(=O)c1ccc(F)c(OCCO)c1]
the method comprising testing a sample from the subject for the presence of elevated Lysosomal lipase A (LIPA) expression and when the sample indicates the presence of elevated LIPA or increased activity of LIPA, the subject is treatable by a composition comprising the compound of Formula II.

102. The method of claim 101, wherein the method further comprises administering a therapeutically-effective amount of the composition.

103. The method of claim 102, wherein the amount of the compound in the composition is sufficient to induce endoplasmic reticulum stress.

104. The method of claim 102 or claim 103, wherein, wherein the amount of the compound in the composition is sufficient to inhibit activity of LIPA.

105. The method of any one of claims 102 to 104, wherein the amount of the compound in the composition is sufficient to shut down protein synthesis.


SEQUENCE LISTING (62 sequences)

SEQ ID NO: 1 - DNA, 21 bp, synthetic construct
  source 1..21: mol_type = other DNA
ctgagtccgaatcaggtgcag

SEQ ID NO: 2 - DNA, 21 bp, synthetic construct
  source 1..21: mol_type = other DNA
atccatggggagatgttctgg

SEQ ID NO: 3 - DNA, 18 bp, synthetic construct
  source 1..18: mol_type = other DNA
ggatttggtcgtattggg

SEQ ID NO: 4 - DNA, 19 bp, synthetic construct
  source 1..19: mol_type = other DNA
ggaagatggtgatgggatt

SEQ ID NO: 5 - DNA, 25 bp, synthetic construct
  source 1..25: mol_type = other DNA
tgttcaaccaattatcagcaaactc

SEQ ID NO: 6 - DNA, 24 bp, synthetic construct
  source 1..24: mol_type = other DNA
ttctgctgtatcctcttcaccagt

SEQ ID NO: 7 - DNA, 22 bp, synthetic construct
  source 1..22: mol_type = other DNA
agaaccaggaaacggaaacaga

SEQ ID NO: 8 - DNA, 20 bp, synthetic construct
  source 1..20: mol_type = other DNA
tctccttcatgcgctgcttt

SEQ ID NO: 9 - DNA, 25 bp, synthetic construct
  source 1..25: mol_type = other DNA
caccgtatcaggaaacacttcacga

SEQ ID NO: 10 - DNA, 25 bp, synthetic construct
  source 1..25: mol_type = other DNA
aaactcgtgaagtgtttcctgatac

SEQ ID NO: 11 - DNA, 25 bp, synthetic construct
  source 1..25: mol_type = other DNA
caccgtcaggcaagctacggtgtgg

SEQ ID NO: 12 - DNA, 25 bp, synthetic construct
  source 1..25: mol_type = other DNA
aaacccacaccgtagcttgcctgac

SEQ ID NO: 13 - DNA, 25 bp, synthetic construct
  source 1..25: mol_type = other DNA
caccgttccccaccatcgtggagcg

SEQ ID NO: 14 - DNA, 25 bp, synthetic construct
  source 1..25: mol_type = other DNA
aaaccgctccacgatggtggggaac

SEQ ID NO: 15 - DNA, 25 bp, synthetic construct
  source 1..25: mol_type = other DNA
caccgctgaagtggatcttcgtcgg

SEQ ID NO: 16 - DNA, 25 bp, synthetic construct
  source 1..25: mol_type = other DNA
aaacccgacgaagatccacttcagc

SEQ ID NO: 17 - DNA, 25 bp, synthetic construct
  source 1..25: mol_type = other DNA
caccggccaccaccactactcatgg

SEQ ID NO: 18 - DNA, 25 bp, synthetic construct
  source 1..25: mol_type = other DNA
aaacccatgagtagtggtggtggcc

SEQ ID NO: 19 - DNA, 25 bp, synthetic construct
  source 1..25: mol_type = other DNA
caccgaatgcatgcggtctatccgt

SEQ ID NO: 20 - DNA, 25 bp, synthetic construct
  source 1..25: mol_type = other DNA
aaacacggatagaccgcatgcattc

SEQ ID NO: 21 - DNA, 25 bp, synthetic construct
  source 1..25: mol_type = other DNA
caccgtgatgggtccatgataaccg

SEQ ID NO: 22 - DNA, 25 bp, synthetic construct
  source 1..25: mol_type = other DNA
aaaccggttatcatggacccatcac

SEQ ID NO: 23 - DNA, 25 bp, synthetic construct
  source 1..25: mol_type = other DNA
caccgttaaccgaattcctcatggg

SEQ ID NO: 24 - DNA, 25 bp, synthetic construct
  source 1..25: mol_type = other DNA
aaaccccatgaggaattcggttaac

SEQ ID NO: 25 - DNA, 25 bp, synthetic construct
  source 1..25: mol_type = other DNA
caccgcaagaacaagtgtattatgt

SEQ ID NO: 26 - DNA, 25 bp, synthetic construct
  source 1..25: mol_type = other DNA
aaacacataatacacttgttcttgc

SEQ ID NO: 27 - DNA, 25 bp, synthetic construct
  source 1..25: mol_type = other DNA
caccgaaaagaagaccaacgtcaca

SEQ ID NO: 28 - DNA, 25 bp, synthetic construct
  source 1..25: mol_type = other DNA
aaactgtgacgttggtcttcttttc

SEQ ID NO: 29 - DNA, 25 bp, synthetic construct
  source 1..25: mol_type = other DNA
caccggtgccatcgagctcatagag

SEQ ID NO: 30 - DNA, 25 bp, synthetic construct
  source 1..25: mol_type = other DNA
aaacctctatgagctcgatggcacc

SEQ ID NO: 31 - DNA, 25 bp, synthetic construct
  source 1..25: mol_type = other DNA
caccgccagcttcatcaccaggcgt

SEQ ID NO: 32 - DNA, 25 bp, synthetic construct
  source 1..25: mol_type = other DNA
aaacacgcctggtgatgaagctggc

SEQ ID NO: 33 - DNA, 25 bp, synthetic construct
  source 1..25: mol_type = other DNA
caccgagcatgggtgccaatcatcg

SEQ ID NO: 34 - DNA, 25 bp, synthetic construct
  source 1..25: mol_type = other DNA
aaaccgatgattggcacccatgctc

SEQ ID NO: 35 - DNA, 25 bp, synthetic construct
  source 1..25: mol_type = other DNA
caccgcagcagaactctcacgacca

SEQ ID NO: 36 - DNA, 25 bp, synthetic construct
  source 1..25: mol_type = other DNA
aaactggtcgtgagagttctgctgc

SEQ ID NO: 37 - DNA, 25 bp, synthetic construct
  source 1..25: mol_type = other DNA
caccgcacatccctgacccaacctg

SEQ ID NO: 38 - DNA, 25 bp, synthetic construct
  source 1..25: mol_type = other DNA
aaaccaggttgggtcagggatgtgc

SEQ ID NO: 39 - DNA, 25 bp, synthetic construct
  source 1..25: mol_type = other DNA
caccgttagctgaatttagtacccg

SEQ ID NO: 40 - DNA, 25 bp, synthetic construct
  source 1..25: mol_type = other DNA
aaaccgggtactaaattcagctaac

SEQ ID NO: 41 - DNA, 25 bp, synthetic construct
  source 1..25: mol_type = other DNA
caccgtccctagagacacctagtaa

SEQ ID NO: 42 - DNA, 25 bp, synthetic construct
  source 1..25: mol_type = other DNA
aaacttactaggtgtctctagggac

SEQ ID NO: 43 - DNA, 25 bp, synthetic construct
  source 1..25: mol_type = other DNA
caccggaccagcaataccctagaga

SEQ ID NO: 44 - DNA, 25 bp, synthetic construct
  source 1..25: mol_type = other DNA
aaactctctagggtattgctggtcc

SEQ ID NO: 45 - DNA, 36 bp, synthetic construct
  source 1..36: mol_type = other DNA
cctcacggaagaaagaaccattctgacaaaggtccc

SEQ ID NO: 46 - DNA, 33 bp, synthetic construct
  source 1..33: mol_type = other DNA
ctttcttccgtgaggaattcggttaaggcacag

SEQ ID NO: 47 - DNA, 36 bp, synthetic construct
  source 1..36: mol_type = other DNA
gagcaggtctactacgtgggtcattctcaaggcacc

SEQ ID NO: 48 - DNA, 37 bp, synthetic construct
  source 1..37: mol_type = other DNA
gtagtagacctgctcttggccagttttattcagaatg

SEQ ID NO: 49 - DNA, 34 bp, synthetic construct
  source 1..34: mol_type = other DNA
tggaaatccctgttttcttctgtgtggatttaat

SEQ ID NO: 50 - DNA, 28 bp, synthetic construct
  source 1..28: mol_type = other DNA
aaacagggatttccacagagctccttca

SEQ ID NO: 51 - DNA, 34 bp, synthetic construct
  source 1..34: mol_type = other DNA
ctgttttcctctgtgtggatttaatgagagaaat

SEQ ID NO: 52 - DNA, 31 bp, synthetic construct
  source 1..31: mol_type = other DNA
cacagaggaaaacagagatttccacagagct

SEQ ID NO: 53 - DNA, 34 bp, synthetic construct
  source 1..34: mol_type = other DNA
gttttcctccgtgtggatttaatgagagaaattt

SEQ ID NO: 54 - DNA, 33 bp, synthetic construct
  source 1..33: mol_type = other DNA
cacacggaggaaaacagagatttccacagagct

SEQ ID NO: 55 - DNA, 39 bp, synthetic construct
  source 1..39: mol_type = other DNA
tccctgttttcctccgtgtggatttaatgagagaaattt

SEQ ID NO: 56 - DNA, 36 bp, synthetic construct
  source 1..36: mol_type = other DNA
ggaggaaaacagggatttccacagagctccttcagt

SEQ ID NO: 57 - DNA, 36 bp, synthetic construct
  source 1..36: mol_type = other DNA
gaaatgtcttctttcttctgtgtggatttaatgaga

SEQ ID NO: 58 - DNA, 32 bp, synthetic construct
  source 1..32: mol_type = other DNA
gaaagaagacatttccacagagctccttcagt

SEQ ID NO: 59 - DNA, 32 bp, synthetic construct
  source 1..32: mol_type = other DNA
tctgtggaggatttaatgagagaaatttaaat

SEQ ID NO: 60 - DNA, 26 bp, synthetic construct
  source 1..26: mol_type = other DNA
taaatcctccacagagctccttcagt

SEQ ID NO: 61 - DNA, 27 bp, synthetic construct
  source 1..27: mol_type = other DNA
acatgttatactggagccaggctgtta

SEQ ID NO: 62 - DNA, 32 bp, synthetic construct
  source 1..32: mol_type = other DNA
tccagtataacatgttttgcacagaagttcca